SEQUENCE LISTING (24 sequences)

SEQ ID NO: 1 - DNA, 62 bp, Artificial Sequence
  Description of Artificial Sequence Synthetic oligonucleotide
agggaaaucaggcuuuaucgggcgccgcucccugugccaucguccgagaguagguagucugt

SEQ ID NO: 2 - RNA, 61 nt, Artificial Sequence
  Description of Artificial Sequence Synthetic oligonucleotide
agggaaaucaggcuuuaucgggcgccgcucccugugccaucguccgagaguagguagucug

SEQ ID NO: 3 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 5 - RNA, 84 nt, Artificial Sequence
  Description of Artificial Sequence Synthetic oligonucleotide
ggagggaaaaguuaucaggcuucuaucgggcgccgcguuuucccuguguuagccaucguccgacgaguaggcuagucugaugcc

SEQ ID NO: 6 - RNA, 19 nt, Artificial Sequence
  Description of Artificial Sequence Synthetic probe
aagccugauuucccuuuuu

SEQ ID NO: 7 - RNA, 18 nt, Artificial Sequence
  Description of Artificial Sequence Synthetic probe
agccugauuucccuuuuu

SEQ ID NO: 8 - RNA, 17 nt, Artificial Sequence
  Description of Artificial Sequence Synthetic probe
gccugauuucccuuuuu

SEQ ID NO: 9 - RNA, 16 nt, Artificial Sequence
  Description of Artificial Sequence Synthetic probe
ccugauuucccuuuuu

SEQ ID NO: 10 - RNA, 15 nt, Artificial Sequence
  Description of Artificial Sequence Synthetic probe
cugauuucccuuuuu

SEQ ID NO: 11 - RNA, 14 nt, Artificial Sequence
  Description of Artificial Sequence Synthetic probe
ugauuucccuuuuu

SEQ ID NO: 12 - RNA, 13 nt, Artificial Sequence
  Description of Artificial Sequence Synthetic probe
gauuucccuuuuu

SEQ ID NO: 13 - RNA, 12 nt, Artificial Sequence
  Description of Artificial Sequence Synthetic probe
auuucccuuuuu

SEQ ID NO: 14 - RNA, 16 nt, Artificial Sequence
  Description of Artificial Sequence Synthetic probe
uuuucagacuaccuac

SEQ ID NO: 15 - RNA, 15 nt, Artificial Sequence
  Description of Artificial Sequence Synthetic probe
uuuucagacuaccua

SEQ ID NO: 16 - RNA, 14 nt, Artificial Sequence
  Description of Artificial Sequence Synthetic probe
uuuucagacuaccu

SEQ ID NO: 17 - RNA, 13 nt, Artificial Sequence
  Description of Artificial Sequence Synthetic probe
uuuucagacuacc

SEQ ID NO: 18 - RNA, 12 nt, Artificial Sequence
  Description of Artificial Sequence Synthetic probe
uuuucagacuac

SEQ ID NO: 19 - RNA, 11 nt, Artificial Sequence
  Description of Artificial Sequence Synthetic probe
uuuucagacua

SEQ ID NO: 20 - RNA, 61 nt, Artificial Sequence
  Description of Artificial Sequence Synthetic oligonucleotide
agggaaaucaggcuuuaucggccgccgcucccugugccaucguccgagaguagguagucug

SEQ ID NO: 21 - RNA, 61 nt, Artificial Sequence
  Description of Artificial Sequence Synthetic oligonucleotide
agggaaaucaggcuuuaucgggagccgcucccugugccaucguccgagaguagguagucug

SEQ ID NO: 22 - RNA, 61 nt, Artificial Sequence
  Description of Artificial Sequence Synthetic oligonucleotide
agggaaaucaggcuuuaucgggcaccgcucccugugccaucguccgagaguagguagucug

SEQ ID NO: 23 - RNA, 61 nt, Artificial Sequence
  Description of Artificial Sequence Synthetic oligonucleotide
agggaaaucaggcuuuaucgggcgccgcucccugugccaucguccgagagucgguagucug

SEQ ID NO: 24 - RNA, 61 nt, Artificial Sequence
  Description of Artificial Sequence Synthetic oligonucleotide
agggaaaucaggcuuuaucgggcgccgcuaccugugccaucguccgagaguagguagucug

(19) United States
(12) Patent Application Publication
(10) Pub. No.: US 2017/0166894 A1
(43) Pub. Date: Jun. 15, 2017

(54) APTAMERS FOR TOPICAL DELIVERY

(71) Applicant: GlaxoSmithKline Intellectual Property Development Limited, Brentford, Middlesex (GB)

(72) Inventors: Kellie Marie DEMOCK, Whitmore Lake, MI (US); Christine Patricia DONAHUE, Waltham, MA (US); Robert L. HALE, Waltham, MA (US); Jon LENN, Durham, NC (US); Jennifer NELSON, Brookline, MA (US); P. Shannon PENDERGRAST, Cambridge, MA (US)

(73) Assignee: GlaxoSmithKline Intellectual Property Development Limited, Brentford, Middlesex (GB)

(21) Appl. No.: 15/124,140

(22) PCT Filed: Mar. 17, 2015
(86) PCT No.: PCT/IB2015/051964

§ 371 Date: Sep. 7, 2016

Related U.S. Application Data
(60) Provisional application No. 61/953,953, filed on Mar. 17, 2014.

(51) Int. Cl. C12N 15/115; A61K 9/107; A61K 9/06; A61K 31/713; A61K 9/00

(52) U.S. Cl. CPC C12N 15/115; A61K 31/713; A61K 9/0014; A61K 47/48092; A61K 9/06; A61K 9/1075; C12N 2320/32; C12N 2310/16; C12N 2310/321; C12N 2310/322; C12N 2310/335; C12N 2320/35; C12N 2310/333; C12N 2310/334; C12N 2310/336

(57) ABSTRACT

Aptamers for topical delivery and methods for topical use of aptamers are described. Aptamers that bind to interleukin (IL)-23 (IL-23 aptamers) and methods of using such aptamers are also described.

### CROSS REFERENCE TO RELATED APPLICATIONS

This application claims priority to U.S. Application Ser. No. 61/953,953, filed on Mar. 17, 2014. The disclosure of the prior application is considered part of (and is incorporated by reference in) the disclosure of this application.

### BACKGROUND OF THE INVENTION

Aptamers are synthetically derived nucleic acid molecules having specific binding affinity to molecules through interactions other than classic Watson-Crick base pairing. Aptamers have a number of desirable characteristics for use as therapeutics and diagnostics including high specificity and affinity, biological efficacy, and excellent pharmacokinetic properties.

### SUMMARY OF THE INVENTION

The skin is the major organ in the human body serving as the interface with the external environment, functioning as a semi permeable barrier to both protect the body from entrance of foreign substances while allowing for loss of excessive endogenous material like water. The skin is the most easily accessible organ in the human body, lending itself to topical and transdermal delivery of pharmaceutical agents. Some of the advantages of topical and drug delivery systems are the avoidance of first pass metabolism, convenience of application, ease of visual assessment, delivery of the pharmaceutical ingredients directly to the site of action, avoidance of the variations seen from systemic deliveries (e.g. gastric emptying, presence of enzymes, pH changes, etc.), ability to easily terminate the medications if safety signals arise, utilisation of highly potent compounds, and suitability for self-medication (Brown et al. Drug Deliv. (2006) 13(3):175-187). However, the skin presents a formidable barrier for topical delivery, especially for compounds that have a molecular weight above 500 daltons. Very few, if any, compounds with a molecular weight above 1,000 daltons have shown clinical efficacy from topical delivery without physical disruption of the barrier. Overcoming this size exclusion phenomenon could present a major breakthrough in dermatology and topical drug delivery as it could open up new areas of research and potentially targets that are not accessible using traditional small molecules.

In some aspects, the disclosure provides a method of treating a skin disease in a subject (e.g., a mammalian subject, e.g., a human), the method comprising topically administering (e.g., applying) a dose (e.g., effective dose, e.g., therapeutically effective dose) of an aptamer (e.g., a pharmaceutically acceptable composition comprising the aptamer) to skin of the subject that comprises the skin disease, (wherein the skin comprises epidermis and dermis), wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis of the skin (e.g., within 15 minutes, 30 minutes, or within 1, 2, 3, 4, 6, 12, 15, 24, 48, or 72 hours of administration).

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments the aptamer comprises cargo (e.g., attached (e.g., conjugated) thereto), e.g., a small molecule, an imaging dye, a fluorophore, or a radiolabel.

In some aspects, the disclosure provides a method for topically administering an aptamer to a subject (e.g., a mammalian subject, e.g., a human), the method comprising:

administering a dose (e.g., effective dose, e.g., therapeutically effective dose) of aptamer (e.g., a pharmaceutically acceptable composition comprising the aptamer) to skin of the subject (wherein the skin comprises epidermis and dermis);

wherein the aptamer enters (e.g., permeates) into the epidermis of the skin; and

wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis (e.g., 15 minutes, 30 minutes, or within 1, 2, 3, 4, 6, 12, 15, 24, 48, or 72 hours of administration);

thereby topically administering the aptamer to the subject.

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin comprises a skin disease.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

In some aspects, the disclosure provides a method of treating a skin disease in a subject (e.g., a mammalian subject, e.g., a human), the method comprising topically administering (e.g., applying) a dose (e.g., effective dose, e.g., therapeutically effective dose) of an aptamer (e.g., a pharmaceutically acceptable composition comprising the aptamer) to skin of the subject (wherein the skin comprises epidermis and dermis), wherein the aptamer has intrinsic activity (e.g., elicits a pharmacodynamic response) in the skin.

In some embodiments, the intrinsic activity is in the epidermis.

In some embodiments, the intrinsic activity is in the dermis.

In some embodiments, the intrinsic activity is measured as an EC50 value (e.g., against a target of interest).

In some embodiments, the intrinsic activity is measured as an IC50 value (e.g., against a target of interest).

In some embodiments, the aptamer is present in the epidermis at a level at least ten-fold (e.g., at least 10-, 100-, 200-, 500-, or 1000-fold) above the IC50 of the aptamer.

In some embodiments, the aptamer is present in the dermis at a level at least ten-fold (e.g., at least 10-, 100-, 200-, 500-, or 1000-fold) above the IC50 of the aptamer.

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

In some aspects, the disclosure provides a method for topically delivering cargo to a subject, the method comprising:

administering an aptamer (e.g., a dose of a pharmaceutically acceptable composition comprising an aptamer) to skin of the subject, wherein the skin comprises epidermis and dermis;

wherein cargo is attached to the aptamer;

wherein the aptamer and attached cargo enter into the epidermis of the skin;

thereby topically delivering cargo to the subject.

In some embodiments, at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose of aptamer and attached cargo enter into the epidermis.

In some embodiments, the cargo (e.g., attached (e.g., conjugated) to the aptamer), comprises a small molecule, an imaging dye, a fluorophore, or a radiolabel.

In some embodiments, the percentage of the applied dose of topically administered aptamer and attached cargo that enters the epidermis is determined in an in vitro assay using ex vivo human skin (e.g., in a Franz cell set up or flow through diffusion cell) (e.g., at 32° C.) (e.g., as described herein).

In some embodiments, the aptamer and attached cargo enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer and attached cargo that enters the dermis is determined in an in vitro assay using ex vivo human skin (e.g., in a Franz cell set up or flow through diffusion cell) (e.g., at 32° C.) (e.g., as described herein).

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer and attached cargo in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer and attached cargo that enters the epidermis is determined in an in vitro assay using ex vivo human skin (e.g., in a Franz cell set up or flow through diffusion cell) (e.g., at 32° C.) (e.g., as described herein).

In some embodiments, the aptamer and attached cargo enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer and attached cargo that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer and attached cargo reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin is normal skin.

In some embodiments, the skin is compromised skin.

In some embodiments, the compromised skin is diseased skin.

In some embodiments, the compromised skin comprises an altered barrier.

In some aspects, the disclosure provides a dose (e.g., effective dose, e.g., therapeutically effective dose) of an aptamer (e.g., a pharmaceutically acceptable composition comprising the aptamer) for use in treating a skin disease in a subject (e.g., a mammalian subject, e.g., a human), wherein the dose is topically administered (e.g., applied) to skin of the subject that comprises the skin disease; (wherein the skin comprises epidermis and dermis); and wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis of the skin (e.g., within 15 minutes, 30 minutes, or within 1, 2, 3, 4, 6, 12, 15, 24, 48, or 72 hours of administration).

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments the aptamer comprises cargo (e.g., attached (e.g., conjugated) thereto), e.g., a small molecule, an imaging dye, a fluorophore, or a radiolabel.

In some aspects, the disclosure provides use of a dose (e.g., effective dose, e.g., therapeutically effective dose) of an aptamer (e.g., a pharmaceutically acceptable composition comprising the aptamer) for the preparation of a medicament for the topical treatment of a skin disease in a subject (e.g., a mammalian subject, e.g., a human), (wherein the skin comprises epidermis and dermis); wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis of the skin (e.g., within 15 minutes, 30 minutes, or within 1, 2, 3, 4, 6, 12, 15, 24, 48, or 72 hours of administration), and wherein the dose is for topical administration (e.g., application) to skin of the subject that comprises the skin disease.

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments the aptamer comprises cargo (e.g., attached (e.g., conjugated) thereto), e.g., a small molecule, an imaging dye, a fluorophore, or a radiolabel.

In some aspects, the disclosure provides a dose (e.g., effective dose, e.g., therapeutically effective dose) of an aptamer (e.g., a pharmaceutically acceptable composition comprising the aptamer) for use in topical administration to a subject (e.g., a mammalian subject, e.g., a human), wherein the dose of aptamer is administered to skin of the subject; (wherein the skin comprises epidermis and dermis); wherein the aptamer enters (e.g., permeates) into the epidermis of the skin; and wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis (e.g., 15 minutes, 30 minutes, or within 1, 2, 3, 4, 6, 12, 15, 24, 48, or 72 hours of administration).

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin comprises a skin disease.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

In some aspects, the disclosure provides use of a dose (e.g., effective dose, e.g., therapeutically effective dose) of an aptamer (e.g., a pharmaceutically acceptable composition comprising the aptamer) for the preparation of a medicament for topical administration to a subject (e.g., a mammalian subject, e.g., a human), (wherein the skin comprises epidermis and dermis); wherein the aptamer enters (e.g., permeates) into the epidermis of the skin; and wherein at least 0.01% (e.g., at least 0.05%, at least 0.1%, at least 0.5%, at least 1%, at least 5%, at least 10%, at least 15%, at least 20%, at least 30%, at least 40%, or at least 50%) of the applied dose enters (e.g., permeates) into the epidermis (e.g., 15 minutes, 30 minutes, or within 1, 2, 3, 4, 6, 12, 15, 24, 48, or 72 hours of administration).

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin comprises a skin disease.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

In some aspects, the disclosure provides a dose (e.g., effective dose, e.g., therapeutically effective dose) of an aptamer (e.g., a pharmaceutically acceptable composition comprising the aptamer) for use in treating a skin disease in a subject (e.g., a mammalian subject, e.g., a human), wherein the dose is topically administered (e.g., applied) to skin of the subject that comprises the skin disease; (wherein the skin comprises epidermis and dermis); and wherein the aptamer has intrinsic activity (e.g., elicits a pharmacodynamic response) in the skin.

In some embodiments, the intrinsic activity is in the epidermis.

In some embodiments, the intrinsic activity is in the dermis.

In some embodiments, the intrinsic activity is measured as an EC50 value (e.g., against a target of interest).

In some embodiments, the intrinsic activity is measured as an IC50 value (e.g., against a target of interest).

In some embodiments, the aptamer is present in the epidermis at a level at least ten-fold (e.g., at least 10-, 100-, 200-, 500-, or 1000-fold) above the IC50 of the aptamer.

In some embodiments, the aptamer is present in the dermis at a level at least ten-fold (e.g., at least 10-, 100-, 200-, 500-, or 1000-fold) above the IC50 of the aptamer.

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

In some aspects, the disclosure provides use of a dose (e.g., effective dose, e.g., therapeutically effective dose) of aptamer (e.g., a pharmaceutically acceptable composition comprising the aptamer) for the preparation of a medicament for topical treatment of a skin disease in a subject (e.g., a mammalian subject, e.g., a human); (wherein the skin comprises epidermis and dermis); wherein the aptamer has intrinsic activity (e.g., elicits a pharmacodynamic response) in the skin; and wherein the dose is for topical administration (e.g., application) to skin of the subject that comprises the skin disease.

In some embodiments, the intrinsic activity is in the epidermis.

In some embodiments, the intrinsic activity is in the dermis.

In some embodiments, the intrinsic activity is measured as an EC50 value (e.g., against a target of interest).

In some embodiments, the intrinsic activity is measured as an IC50 value (e.g., against a target of interest).

In some embodiments, the aptamer is present in the epidermis at a level at least ten-fold (e.g., at least 10-, 100-, 200-, 500-, or 1000-fold) above the IC50 of the aptamer.

In some embodiments, the aptamer is present in the dermis at a level at least ten-fold (e.g., at least 10-, 100-, 200-, 500-, or 1000-fold) above the IC50 of the aptamer.

In some embodiments, the aptamer is present in a pharmaceutically acceptable composition (e.g., a vehicle) (e.g., a vehicle described herein, e.g., a vehicle presented in Table 4 or 9).

In some embodiments, the concentration of the aptamer in the pharmaceutically acceptable composition is between 0.001% and 20% (e.g., between 0.001% and 15%, between 0.001% and 10%, between 0.001% and 5%, between 0.001% and 2%, between 0.01% and 20%, between 0.01% and 10%, between 0.01% and 5%, between 0.01% and 2%, between 0.1% and 20%, between 0.1% and 15%, between 0.1% and 10% between, 0.1% and 5%, or between 0.1% and 2%).

In some embodiments, the pharmaceutically acceptable composition is administered as a spray (e.g., aerosol), a liquid, an ointment, a cream, a lotion, a solution (e.g., an aqueous solution), a suspension, an emulsion, a paste, a gel, a powder, a foam, a slow release nanoparticle, a slow release microparticle, a bioadhesive, a patch, a bandage, a semi-solid dosage form or a wound dressing.

In some embodiments, the pharmaceutically acceptable composition is administered as an aqueous solution.

In some embodiments, the pharmaceutically acceptable composition is administered as a semi-solid dosage form.

In some embodiments, the pharmaceutically acceptable composition is administered as a cream.

In some embodiments, the pharmaceutically acceptable composition does not contain a penetration enhancer (e.g., propylene glycol).

In some embodiments, the applied dose of the pharmaceutically acceptable composition to the skin is between 0.01 and 20 mg/cm² (e.g., between 0.01 and 15 mg/cm², between 0.01 and 10 mg/cm², between 0.01 and 5 mg/cm², between 0.01 and 2 mg/cm², between 0.05 and 20 mg/cm², between 0.05 and 15 mg/cm², between 0.05 and 10 mg/cm², or between 0.05 and 5 mg/cm², between 0.05 and 2 mg/cm², between 0.1 and 20 mg/cm², between 0.1 and 15 mg/cm², between 0.1 and 10 mg/cm², between 0.1 and 5 mg/cm², or between 0.1 and 2 mg/cm²).

In some embodiments, the percentage of the applied dose of topically administered aptamer that enters the epidermis is determined in an in vitro assay using ex vivo human skin (e.g., in a Franz cell set up or flow through diffusion cell) (e.g., at 32° C.) (e.g., as described herein).

In some embodiments, the aptamer enters (e.g., permeates) into the dermis of the skin; and wherein at least 0.01% (e.g., at least 0.05%, at least 0.1%, at least 0.5%, at least 1%, at least 5%, or at least 10%) of the applied dose enters (e.g., permeates) into the dermis (e.g., within 15 minutes, 30 minutes, or within 1, 2, 3, 4, 6, 12, 15, 24, 48, or 72 hours of administration).

In some embodiments, the percentage of the applied dose of topically administered aptamer that enters the dermis is determined in an in vitro assay using ex vivo human skin (e.g., in a Franz cell set up or flow through diffusion cell) (e.g., at 32° C.) (e.g., as described herein).

In some embodiments, less than 5% (e.g., less than 1%, less than 0.5%, less than 0.1%, less than 0.05%, less than 0.01%, less than 0.001%, or below the limit of detection), of the topically applied the aptamer reaches systemic circulation (e.g., after 2, 3, 4, 6, 12, 15, 24, 48 or 72 hours of administration).

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the pharmaceutically acceptable composition is administered once daily.

In some embodiments, the pharmaceutically acceptable composition is administered twice daily.

In some embodiments, the pharmaceutically acceptable composition is administered once weekly.

In some embodiments, the skin disease comprises an exacerbation of asthma, an exacerbation of rheumatoid arthritis, an exacerbation of psoriatic arthritis, an exacerbation of Sjogren's Syndrome, an exacerbation of uveitis, an exacerbation of Graft vs. Host disease (GVHD), an exacerbation of chronic obstructive pulmonary disease (COPD), an exacerbation of arthralgia, or an exacerbation of Islet Cell Transplant inflammation.

In some embodiments, the skin disease comprises psoriasis, dermatitis (e.g., atopic dermatitis, contact dermatitis, eczematous dermatitis, or seborrhoic dermatitis), hidradentis supporativa, Oral Lichen Planus, a chronic blistering (bullous) disorder, acne, seborrhoeic cutaneous manifestations of immunologically-mediated disorders, alopecia, alopecia greata, scleredoma, scar formation (e.g., a keloid scar or a hypertrophic scar), urticaria, rosacea, melanoma, or inflammation from kidney transplant.

In some embodiments, the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the skin disease comprises psoriasis.

In some embodiments, the psoriasis is mild psoriasis.

In some embodiments, the psoriasis is moderate psoriasis.

In some embodiments, the psoriasis is severe psoriasis.

In some embodiments, the skin disease comprises dermatitis (e.g., atopic dermatitis, contact dermatitis, eczematous dermatitis, or seborrhoic dermatitis).

In some embodiments, the pharmaceutically acceptable composition is administered in combination with second agent that comprises a treatment for the skin disease.

In some embodiments, the aptamer binds to a first target and/or a target of the first target, which can include interactions with other biologic or chemical entities that can be subject to modulation (including, e.g., agonism, antagonism, stabilization, destabilization, enhanced clearing from cellular, organ, or whole body systems by aptamers), including soluble ligands and/or their receptors, extracellular, transmembrane, and intracellular enzymes, scaffolding/structural proteins, RNA, DNA, lipids, fatty acids, co-factors and the respective specific and/or non-specific interactions.

In some embodiments, the aptamer binds to a target (e.g., protein) in the skin, e.g., IL2, IL12, IL17, IL22, IL23, IFNγ, TNFα, TSLP, CCR1, CXCR4, CRTH2, aTLR, p53, RAS, MEK, PATCH1, a mediator of sonic hedgehog signaling, miRNA (e.g., mir21), PI3K/AKT, PTEN, FAS, DGAT, SCD1, EGFR, EGF, HB-EGF, TGF-α, EPR, BTC, NRG1-4, a β-integrin, CD44, E-cadherin, CLA, HPV1, HPV5, HPV8, HPV14, HPV16, HPV17, HPV20, HPV31, HPV47, VEGFC, VEGFD, VEGFEGF, FGF, GMCSF, IL-1, PDGF, or TGF-β1.

In some embodiments, the aptamer binds to a target in the epidermis. In some embodiments, the aptamer binds to a target in the dermis.

In some embodiments, the skin is normal skin.

In some embodiments, the skin is compromised skin.

In some embodiments, the compromised skin is diseased skin.

In some embodiments, the compromised skin comprises an altered barrier.

In some embodiments, the aptamer comprises cargo (e.g., attached (e.g., conjugated) thereto), e.g., a small molecule, an imaging dye, a fluorophore, or a radiolabel.

The present disclosure provides aptamers that bind to interleukin (IL)-23 (IL-23 aptamers) and methods of use thereof, e.g., for the treatment (e.g., topical treatment) of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)).

In some embodiments, the IL-23 aptamer binds to IL-23 and inhibits IL-23 activity, e.g., inhibits (e.g., decreases, e.g., by about 20%, about 30%, about 40%, about 50%, about 60%, about 70%, about 80%, about 90%, about 95% or more) IL-23 signaling. In some embodiments, the effect of the aptamer on IL-23 activity can be evaluated by evaluating a response that is downstream of IL-23 engagement of the IL-23 receptor (IL-23R), e.g., using a method described herein, e.g., phosphoSTAT3 assay, or other ELISA method to quantify IL-17 (IL-17a), IL-17f, or IL-22 levels, e.g., in PBMCs. In some embodiments, the response evaluated is STAT3 phosphorylation, IL-17 expression, or IL-22 expression, wherein the IL-23 aptamer decreases the response, e.g., as compared to a control (e.g., the level of response under identical conditions but in the absence of the IL-23 aptamer).

In some aspects, the disclosure provides an aptamer that binds to human interleukin (IL)-23, wherein the aptamer comprises a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2.

In some embodiments, the aptamer consists of a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2.

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, the aptamer comprises a secondary structure similar (e.g., identical) to an aptamer consisting of SEQ ID NO:1.

In some embodiments, the aptamer decreases STAT3 phosphorylation, e.g., as determined in an in vitro phosphoSTAT3 assay, e.g., as compared to a control, e.g., under identical conditions except in the absence of aptamer.

In some embodiments, the aptamer decreases IL-17 expression, e.g., as determined by ELISA, e.g., as compared to a control, e.g., under identical conditions except in the absence of aptamer.

In some embodiments, the aptamer comprises SEQ ID NO:1.

In some embodiments, the aptamer consists of SEQ ID NO:1.

In some embodiments, the aptamer comprises SEQ ID NO:2.

In some embodiments, the aptamer consists of SEQ ID NO:2.

In some embodiments, the aptamer binds to human IL-23 as well as (e.g., at least about 75%. about 50%, about 40% about 30%, or about 20% as well) an aptamer consisting of SEQ ID NO:1, e.g., under identical conditions.

In some embodiments, the aptamer inhibits STAT3 phosphorylation as well as (e.g., at least about 75%. about 50%, about 40% about 30%, or about 20% as well) an aptamer consisting of SEQ ID NO:1, e.g., under identical conditions.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some aspects, the disclosure provides a pharmaceutical composition (e.g., pharmaceutically acceptable composition) comprising an aptamer that binds to human interleukin (IL)-23, wherein the aptamer comprises a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2, or a salt thereof; and a pharmaceutically acceptable carrier or diluent.

In some embodiments, the pharmaceutical composition comprises a semi-solid dosage form.

In some embodiments, the pharmaceutical composition comprises a cream.

In some embodiments, the aptamer consists of a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2.

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, the aptamer comprises a secondary structure similar (e.g., identical) to an aptamer consisting of SEQ ID NO:1.

In some embodiments, the aptamer decreases STAT3 phosphorylation, e.g., as determined in an in vitro phosphoSTAT3 assay, e.g., as compared to a control, e.g., under identical conditions except in the absence of aptamer.

In some embodiments, the aptamer decreases IL-17 expression, e.g., as determined by ELISA, e.g., as compared to a control, e.g., under identical conditions except in the absence of aptamer.

In some embodiments, the aptamer comprises SEQ ID NO:1.

In some embodiments, the aptamer consists of SEQ ID NO:1.

In some embodiments, the aptamer comprises SEQ ID NO:2.

In some embodiments, the aptamer consists of SEQ ID NO:2.

In some embodiments, the aptamer binds to human IL-23 as well as (e.g., at least about 75%. about 50%, about 40% about 30%, or about 20% as well) an aptamer consisting of SEQ ID NO:1, e.g., under identical conditions.

In some embodiments, the aptamer inhibits STAT3 phosphorylation as well as (e.g., at least about 75%. about 50%, about 40% about 30%, or about 20% as well) an aptamer consisting of SEQ ID NO:1, e.g., under identical conditions.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some aspects, the disclosure provides a method of treating an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis))) in a subject (e.g., human subject), the method comprising administering an aptamer (e.g., a therapeutically effective amount thereof) (e.g., a pharmaceutical composition (e.g., pharmaceutically acceptable composition) comprising the aptamer) to the subject, wherein the aptamer binds to human interleukin (IL)-23, wherein the aptamer comprises a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2.

In some embodiments, the aptamer consists of a nucleic acid sequence at least 80% (e.g., at least 85%, at least 90%, at least 91%, at least 92%, at least 93%, at least 94%, at least 95%, at least 96%, at least 97%, at least 98%, or at least 99%) identical to a sequence selected from the group consisting of: SEQ ID NO:1 and 2.

In some embodiments, the length of the aptamer is less than 100 (e.g., less than 95, 90, 85, 80, 75, or 70) nucleotides.

In some embodiments, the length of the aptamer is greater than 30 (e.g., greater than 35, 40, 45, 50, 55, 60, or 65) nucleotides.

In some embodiments, the aptamer is between 30 and 90 nucleotides (e.g., between 35 and 85 nucleotides) in length.

In some embodiments, the aptamer comprises a secondary structure similar (e.g., identical) to an aptamer consisting of SEQ ID NO:1.

In some embodiments, the aptamer decreases STAT3 phosphorylation, e.g., as determined in an in vitro phosphoSTAT3 assay, e.g., as compared to a control, e.g., under identical conditions except in the absence of aptamer.

In some embodiments, the aptamer decreases IL-17 expression, e.g., as determined by ELISA, e.g., as compared to a control, e.g., under identical conditions except in the absence of aptamer.

In some embodiments, the aptamer comprises SEQ ID NO:1.

In some embodiments, the aptamer consists of SEQ ID NO:1.

In some embodiments, the aptamer comprises SEQ ID NO:2.

In some embodiments, the aptamer consists of SEQ ID NO:2.

In some embodiments, the aptamer binds to human IL-23 as well as (e.g., at least about 75%. about 50%, about 40% about 30%, or about 20% as well) an aptamer consisting of SEQ ID NO:1, e.g., under identical conditions.

In some embodiments, the aptamer inhibits STAT3 phosphorylation as well as (e.g., at least about 75%. about 50%, about 40% about 30%, or about 20% as well) an aptamer consisting of SEQ ID NO:1, e.g., under identical conditions.

In some embodiments, at least one nucleotide of the aptamer comprises a chemical modification.

In some embodiments, all nucleotides of the aptamer comprise a chemical modification.

In some embodiments, the chemical modification comprises a modification on the 2′ position of the sugar.

In some embodiments, the chemical modification comprises a 2′-O-methoxyethyl addition.

In some embodiments, the chemical modification comprises a 2′fluoro addition.

In some embodiments, the aptamer comprises a single inverted deoxythymidine residue (idT) on its 3′ terminus.

In some embodiments, the aptamer is administered topically.

In some embodiments, the aptamer is administered once daily.

In some embodiments, the aptamer is administered twice daily.

In some embodiments, the aptamer is administered once weekly.

In some embodiments, the aptamer is in a semi-solid dosage form.

In some embodiments, the aptamer is in an aqueous solution.

In some embodiments, the aptamer is in a cream.

In some embodiments, the inflammatory skin disease (e.g., chronic inflammatory skin disease) comprises psoriasis.

In some embodiments, the psoriasis is mild psoriasis.

In some embodiments, the psoriasis is moderate psoriasis.

In some embodiments, the psoriasis is severe psoriasis.

In some embodiments, the inflammatory skin disease (e.g., chronic inflammatory skin disease) comprises atopic dermatitis.

In some embodiments, the aptamer is administered in combination with second treatment for the inflammatory skin disease (e.g., chronic inflammatory skin disease).

In some aspects, the disclosure provides a method of detecting an oligonucleotide (e.g., aptamer), the method comprising:

contacting a first probe (e.g., detection probe) with an oligonucleotide (e.g., aptamer), thereby creating a mixture;

heating the mixture to a temperature (e.g., 95° C.) that results in unfolding of the oligonucleotide (e.g., aptamer);

cooling the mixture;

allowing the first probe to interact with the oligonucleotide (e.g., aptamer) (e.g., via complementary base pairing) (e.g., providing conditions that allow the first probe to interact with the oligonucleotide), thereby forming a probe:oligonucleotide complex (e.g., probe:aptamer complex);

contacting the mixture with a second probe (e.g., capture probe), wherein the contacting occurs under conditions that allow the second probe to interact with the oligonucleotide (e.g., aptamer) of the probe:oligonucleotide complex (e.g., probe:aptamer complex) (e.g., via complimentary base pairing), thereby forming a captured complex; and

detecting the captured complex.

In some embodiments, the method further comprises quantitating the amount of oligonucleotide (e.g., aptamer) on the surface, e.g., in the captured complex.

In some embodiments, the second probe comprises a free amine on its 5′ terminus.

In some embodiments, the second probe is linked (e.g., covalently linked) to a surface via the free amine.

In some embodiments, the second probe is linked (e.g., covalently linked) to a surface (e.g., plate or bead).

In some embodiments, the first probe comprises a detectable label (e.g., biotin) on its 3′ terminus.

In some embodiments, detecting the captured complex comprises detecting the detectable label on the surface (e.g., using strepavidin to bind to the biotin, e.g., the strepavidin is conjugated to a reagent that can be detected and/or quantified, or the reagent (e.g., horseradish peroxidase or alkaline phosphatase) acts on a substrate (e.g., tetramethylbenzidine or p-nitrophenyl phosphate) that can be detected and/or quantified, e.g., in a colorimetric assay).

In some embodiments, the method further comprises a wash step prior to the detecting step.

In some embodiments, the second probe comprises a spacer (e.g., a 3, 4, or 5 nucleotide spacer) (e.g., 4 nucleotide spacer) between the free amine and the 5′ terminus of the nucleotides that provide interaction (e.g., complementary base pairing) with the oligonucleotide (e.g., aptamer).

In some embodiments, the first probe comprises a spacer (e.g., a 3, 4, or 5 nucleotide spacer) (e.g., 4 nucleotide spacer) between the detectable label (e.g., biotin) and the 3′ terminus of the nucleotides that provide interaction (e.g., complementary base pairing) with the oligonucleotide (e.g., aptamer).

In some aspects, the disclosure provides a method of detecting an oligonucleotide (e.g., aptamer), the method comprising:

contacting a first probe (e.g., detection probe) with an oligonucleotide (e.g., aptamer), thereby creating a mixture, wherein the contacting occurs under conditions that allow the first probe to interact with the oligonucleotide (e.g., aptamer), thereby forming a probe:oligonucleotide complex (e.g., probe:aptamer complex);

contacting the mixture with a second probe (e.g., capture probe), wherein the contacting occurs under conditions that allow the second probe to interact with the oligonucleotide (e.g., aptamer) of the probe:oligonucleotide complex (e.g., probe:aptamer complex), thereby forming a captured complex; and

detecting the captured complex.

In some embodiments, the interacting comprises complimentary base pairing.

In some embodiments, the method further comprises quantitating the amount of oligonucleotide (e.g., aptamer) on the surface, e.g., in the captured complex.

In some embodiments, the method further comprises a wash step the mixture prior to the detecting step.

In some embodiments, the first probe comprises a detectable label (e.g., biotin) on its 3′ terminus.

In some embodiments, the second probe comprises a free amine on its 5′ terminus.

In some embodiments, the second probe is linked (e.g., covalently linked) to a surface via the free amine.

In some embodiments, the second probe is linked (e.g., covalently linked) to a surface.

In some embodiments, the first probe comprises a detectable label (e.g., biotin) on its 3′ terminus.

In some embodiments, detecting the captured complex comprises detecting the detectable label on the surface (e.g., using strepavidin to bind to the biotin, e.g., the strepavidin is conjugated to a reagent that can be detected and/or quantified, or the reagent (e.g., horseradish peroxidase or alkaline phosphatase) acts on a substrate (e.g., tetramethylbenzidine or p-nitrophenyl phosphate) that can be detected and/or quantified, e.g., in a colorimetric assay).

In some embodiments, the second probe comprises a spacer (e.g., a 3, 4, or 5 nucleotide spacer) (e.g., 4 nucleotide spacer) between the free amine and the 5′ terminus of the nucleotides that provide interaction (e.g., complementary base pairing) with the oligonucleotide (e.g., aptamer).

In some embodiments, the first probe comprises a spacer (e.g., a 3, 4, or 5 nucleotide spacer) (e.g., 4 nucleotide spacer) between the detectable label (e.g., biotin) and the 3′ terminus of the nucleotides that provide interaction (e.g., complementary base pairing) with the oligonucleotide (e.g., aptamer).

In some aspects, the disclosure provides aptamers (e.g., for topical delivery) that comprise at least one chemical modification. In one embodiment, the modification is selected from the group consisting of: a chemical substitution at a sugar position; a chemical substitution at a phosphate position; and a chemical substitution at a base position, of the nucleic acid; incorporation of a modified nucleotide; 3′ capping (e.g., 3′-idT); conjugation to a high molecular weight, non-immunogenic compound; conjugation to a lipophilic compound; and phosphate backbone modification. In one embodiment, the non-immunogenic, high molecular weight compound conjugated to the aptamer of the disclosure is polyalkylene glycol, preferably polyethylene glycol. In one embodiment, the backbone modification comprises incorporation of one or more phosphorothioates into the phosphate backbone.

In some aspects, the disclosure provides a pharmaceutical composition comprising (e.g., a therapeutically effective amount of) an aptamer comprising a nucleic acid sequence selected from the group consisting of: SEQ ID NO:1 or 2, or a salt thereof, and a pharmaceutically acceptable carrier or diluent.

In some aspects, the disclosure features an aptamer described herein for topical use in the treatment of a skin disease, wherein the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some aspects, the disclosure provides the use of an aptamer described herein for the preparation of a medicament for the topical treatment of a skin disease, wherein the skin disease comprises a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some aspects, the disclosure features an aptamer described herein for topical use in the treatment of a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation), as described herein.

In some aspects, the disclosure provides the use of an aptamer described herein for the preparation of a medicament for the topical treatment of a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation), as described herein.

In some aspects, the disclosure features an aptamer described herein for use in the treatment of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis))), as described herein.

In some aspects, the disclosure provides the use of an aptamer described herein for the preparation of a medicament for the treatment of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis))), as described herein.

### BRIEF DESCRIPTION OF THE DRAWINGS

FIGS. 1A and 1B. FIG. 1A is a schematic showing the putative secondary structure of ARC32225 (SEQ ID NO:1). ARC32225 is comprised of 2′-methoxy (circles) and 2′-fluoro nucleotides and is modified at its 3′-terminus with an inverted deoxythymidine residue (idT) to increase nuclease resistance. FIG. 1B is a schematic showing a tertiary contact in ARC32225. Also shown are IC50 values from compensatory mutation studies that suggest that the tertiary interactions in the IL23 molecule include a loop-tail contact. Mutations are shown in bold italics.

FIG. 2 is a line graph showing that the minimization and optimization of ARC20122 to ARC32225 does not impact its functional activity.

FIG. 3 is a line graph showing purified endogenous IL-23 phospho-STAT3 induction is inhibited by both p-40 and p-19 neutralizing monoclonal antibodies.

FIG. 4 is a line graph showing ARC32225 inhibition of endogenous IL-23 in the PHA Blast phospho-STAT3 assay.

FIG. 5 is a line graph showing activity of ARC32225 versus eIL-23 (endogenous IL-23) and rIL-12 (recombinant IL-12).

FIG. 6 is a line graph showing determination of appropriate detection probe for DHA for ARC32225.

FIG. 7 is a line graph showing determination of appropriate capture probe for DHA for ARC32225.

FIG. 8 is a line graph showing the sensitivity of the DHA in murine plasma.

FIG. 9 is a line graph showing the sensitivity of the DHA in porcine plasma.

FIG. 10 is a line graph showing the sensitivity of the DHA in cynomolgus plasma.

FIG. 11 is a panel of bar graphs showing skin penetration of IL23 aptamer (ARC32225) in human ex vivo skin.

FIG. 12 is two panels of bar graphs showing formulation enhancements of skin penetration of IL23 aptamer (ARC32225) in human ex vivo skin.

FIGS. 13A and 13B are two panels of line graphs showing IL23 aptamer (ARC32225) in aqueous vehicle over 24 hours measured in the receiving fluid after a single topical application on epidermal skin sections shown as cumulative amount (A) and flux (B).

FIG. 14 is two panels of bar graphs showing passive permeation of IL23 aptamer (ARC32225) into the epidermis and dermis comparing the effects of occlusion, hydration, and finite versus infinite dosing at 6 hours (top panel) and 15 hours (bottom panel) post-application.

FIG. 15 is two panels of bar graphs showing passive permeation of IL23 aptamer (ARC32225) into the epidermis and dermis comparing the effects of calcium chloride and EDTA from a finite dose (10 mg/cm2) at 6 hours (top panel) and 15 hours (bottom panel) post-application.

FIG. 16 is a panel of bar graphs showing passive permeation of IL23 aptamer (ARC32225) comparing the effects of temperature on a finite dose (10 mg/cm2).

FIGS. 17A, 17B, and 17C are two panels of bar graphs showing skin penetration of different 2′ modified IL23 aptamers on ex vivo human skin at 6 hours (A) and 24 hours (B) post application, and a panel showing skin penetration (as percent of applied dose) of four different variants (Variant 1; 1.1 mg/mL, Variant 2; 0.4 mg/mL, Variant 3; 0.9 mg/mL, and Variant 4; 0.9 mg/mL) disrupting secondary structure and one variant (Variant 5; 1.1 mg/mL) disrupting tertiary structure and IL23 aptamer (ARC32225) (1.0 mg/mL) in a water solution at 24 hours (C) post application.

FIGS. 18 A and 18B are two panels of bar graphs showing biological activity in human skin of topical IL23 aptamer (ARC32225), as assessed by a reduction in IL17f (A) and IL22 (B) cytokine levels.

### DETAILED DESCRIPTION OF THE INVENTION

It is generally understood that agents larger than 1,000 daltons cannot pass into intact (e.g., uncompromised) mammalian (e.g., human) skin upon topical delivery, e.g., pass through the stratum corneum (SC) and into the living layers of the epidermis and/or dermis. Overcoming the size exclusion phenomenon of the mammalian skin barrier presents a major breakthrough in dermatology and topical drug delivery. This size exclusion phenomenon, less than 1,000 daltons, is reinforced by the drugs that have become commercially available as transdermal and topical products [1]. The skin has evolved to protect the human body from physical, mechanical, and chemical insults while preventing endogenous water loss. This is achieved by a thin (10-30 um) cornified layer at the outermost layer of the epidermis through the terminal differentiation of the basal keratinocytes to eventually form the stratum corneum. This outermost layer consists of flattened keratin-rich, lipoprotein-containing envelopes surrounded by a lipid envelope that are continually removed through abrasion and replenished approximately every thirty days [2, 3]. This lipid envelope creates cohesion between the corneocytes and the surrounding intercellular lipids, which have a well defined lateral packing and lamellar phases to form this highly effective barrier of protection [4]. It is this highly evolved barrier that both protects the human body but also limits the therapeutic targets achievable from topical dosing.

Aptamers are a relatively new class of oligonucleotide-based technologies that can bind to a wide range of molecules including proteins, nucleotides, antibiotics, low-molecular organic or inorganic compounds, and even whole cells with high affinity and specificity [5]. One major difference between antisense oligonucleotides and aptamers are the conformational structures and flexibility [10]. Structure in antisense molecules is actively avoided because it can have an impact on efficacy. One of the advantages of aptamers is that structure can be modified and still maintain activity. RNA based aptamers have been described to have a conformational ‘plasticity’ creating many different tertiary structures; the RNA's high potential for plasticity may result from the evolution of molecular mimicry between RNA and proteins using the different functionally active protein architecture [6]. It has now been determined that the attributes of plasticity, reversible denaturing (flexibility), and high stability lend themselves to topical delivery to cross the skin barrier. Single stranded aptamers, e.g., with 2′ modified nucleotides, represent a subset of large molecular weight compounds capable of penetrating human skin without physical disruption of the barrier (e.g., intact human skin) (i.e., passive delivery) for the treatment of skin diseases. In addition, antisense oligonucleotides are often about 20 nucleotides in length; in contrast, aptamers are generally larger, e.g, at least 30 nucleotides in length, and often larger, e.g., 50-90 nucleotides.

The present disclosure provides the first description of topical delivery of an aptamer through intact (e.g., uncompromised) human skin. Further, such topically delivered aptamers can exert a therapeutic effect in the skin, e.g., in the epidermis and/or dermis. Although animal models (e.g., rodent, porcine) to study topical delivery exist, the skin of these model systems is generally more permeable than human skin and may not be predictive of effects on human skin, e.g., passage through the stratum corneum (SC) and into the epidermis and/or dermis.

The topical administration of the IL23 aptamer has been shown to penetrate intact human skin viable epidermis and dermis at therapeutically relevant concentrations. The concentrations were quantified using a dual hybridization assay shown to be able to extract and quantify aptamer from human skin at picomolar concentrations. The levels quantified in the epidermis were consistently greater than 1,000-fold above the IC50 (micromolar) in the epidermis and greater than 100-fold above the IC50 (nanomolar to micromolar) in the dermis. In addition, the amount of applied dose of aptamer that permeated into both the epidermis and dermis was calculated. Depending on the vehicle (e.g., pharmaceutically acceptable composition), over 11% of the applied dose permeated into the epidermis and over 1% into the dermis. The levels quantified within the skin were confirmed using fluorescence and shown to be both in and around the keratinocytes (intracellular and extracellular). This suggests the aptamers can target both extracellular targets as well as intracellular or even nuclear targets. The concentrations quantified in the skin were confirmed for biological activity within the skin through cytokine inhibition confirming the extracellular fractions were capable of binding to a protein to elicit a biological response. The levels quantified in the skin were confirmed for biological activity in a pharmacodynamic model using freshly excised human skin.

As described herein, inhibiting IL-23, e.g., by use of an IL-23 aptamer (e.g., topically administered), in skin diseases can block activation and expansion of Th17 lymphocytes in the skin and ameliorate the symptoms of an inflammatory skin disease (e.g., a chronic inflammatory skin disease (e.g., psoriasis or atopic dermatitis)), e.g., without compromising a subject's (e.g., human subject's) systemic immune response.

One aptamer described herein, ARC32225, is an optimized and stabilized 61-nucleotide (MW=20,395.27) mRfY aptamer. The 3′-terminus carries an additional inverted dT cap as protection against nuclease degradation. For topical administration, it is not PEGylated. ARC32225 binds to IL-23 that has been produced by activated human monocytes in vitro (endogenous IL-23 or eIL-23). ARC32225 is a potent inhibitor of STAT3 activation that is induced by endogenous IL-23 but not of STAT3 activation that is induced by IL-12 (as determined in an eIL-23-dependent STAT3 activation in primary human T-cells (IC50=˜300 pM)). Although ARC32225 does not inhibit most non-human IL-23 proteins, it does inhibit cynomolgus macaque IL-23, enabling on-target toxicology studies. ARC32225 is generally resistant to nuclease degradation. A significant amount of full length ARC32225 remains after incubation in 90% human serum for 24 hours. ARC32225 also remains nearly 100% intact when formulated in a cream at room temperature for two weeks. Intact ARC32225 penetrates and localizes in the epidermis for at least four hours in healthy human skin when applied topically in a 1% cream formulation in an in vitro skin penetration study. The aptamer penetrates healthy ex-vivo skin throughout all layers of the epidermis and the upper layer of the dermis. In a preliminary in vivo experiment, ARC32225 penetrated and resided for at least 4 hours in tape stripped live porcine skin. Topical application of an IL-23 aptamer like ARC32225 may decrease Th17-cell activity in the skin, leading to a decrease in the local inflammatory effect while not affecting systemic Th1 or Th17-cell activity, e.g., that is needed for an effective immune response to infection and reducing the risk of cancer.

Skin Structure

Skin may be intact (e.g., healthy or undiseased skin) or may be compromised (e.g., diseased skin or have an altered barrier). Normal (intact) skin means skin with an intact stratum corneum layer that is not affected by disease, has normal barrier functions to protect the body from the environment, and normal skin temperature (typically in the range of 30-36° C.).

Compromised skin is skin that is affected by disease as defined below where the stratum corneum has been damaged, thickened (e.g. hyperkeratosis), compromised, more or less permeable, or that lacks at least some of the stratum corneum (e.g., skin damaged by exposure to an agent; an immune response or exacerbation thereof; an inflammatory response or exacerbation thereof; a physical trauma such as a cut, wound, or abrasion; a underdeveloped skin such as occurs in a preterm infant; conditions in which either all or part of the epidermis is exposed; conditions in which part of the dermis has been removed such as partial thickness wounds encountered in resurfacing procedures such as chemical peels, dermabrasions, and laser resurfacing, etc.).

The skin has two main layers, the epidermis and dermis. Below these is a layer of subcutaneous fat. The outer surface of the skin is the epidermis, which itself contains several layers: the viable (e.g., living) layers (the basal cell layer, the spinous layer, the granular cell layer), and the non-viable stratum corneum outer layer. The deepest layer of the epidermis is the basal cell layer. Here cells are continually dividing to produce new skin cells. These cells move towards the skin surface, pushed upward by the dividing cells below them. Blood vessels in the dermis, which is below the basal cell layer, supply nutrients to support this active growth of new skin cells. As the basal cells move upwards and away from their blood supply, their cell content and shape change. Cells above the basal cell layer become more irregular in shape and form the spinous layer. Above this, cells move into the granular layer. Being distant from the blood supply in the dermis, the cells begin to die and accumulate keratin.

The stratum corneum is the top (non-viable) layer of the epidermis. Cells here are flat and scale-like (‘squamous’) in shape. These cells are dead, contain a lot of keratin and are arranged in overlapping layers that impart a tough and waterproof character to the skin's surface. Dead skin cells are continually shed from the skin's surface. This is balanced by the dividing cells in the basal cell layer, thereby producing a state of constant renewal that occurs approximately every 30 days. Also in the basal cell layer are cells that produce melanin. Melanin is a pigment that is absorbed into the dividing skin cells to help protect them against damage from sunlight (ultraviolet light). The amount of melanin in your skin is determined by genetic makeup and one's exposure to sunlight.

Below the epidermis is the layer called the dermis. The top layer of the dermis, the one directly below the epidermis, has many ridges called papillae. On the fingertips, the skin's surface follows this pattern of ridges to create our individual fingerprints. The dermis contains a variable amount of fat, and also collagen and elastin fibers that provide strength and flexibility to the skin. Blood vessels supply nutrients to the dividing cells in the basal layer and remove any waste products.

Skin Disease:

Topical formulations and/or topical administration of an aptamer may be used for treating a dermatological disorder (skin disease) (i.e., an abnormal state of the epidermis, dermis, and/or subcutaneous tissues). Skin diseases include an inflammatory dermatological disease, that includes a dermatological disease is caused by (either partially or fully) an immune disorder, (e.g. an autoimmune disorder (e.g., eczema, psoriasis, atopic dermatitis)); a proliferation disorder (e.g., melanoma); contact with an allergen and/or an irritant; an overproduction of sebum lipids (e.g., acne); a fibroblast disorder (e.g., scarring after a trauma (e.g., surgery)); or combinations thereof. Examples of dermatological (skin) diseases include, but are not limited to, psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation.

Additional skin diseases that can be treated by topical formulations and/or topical administration of an aptamer include:
Skin disorders of persistent inflammation, cell kinetics, and differentiation (e.g., psoriasis, psoriatic arthritis, exfoliative dermatitis, pityriasis rosea, lichen planus, lichen nitidus, porokeratosis, etc.);
Skin disorders of epidermal cohesion, vesicular and bullous disorders (e.g., pemphigus, bulluous pemphigoi, epidermamolysis bullosa acquisita, pustular eruptions of the palms and soles, etc.);
Skin disorders of epidermal appendages and related disorders (e.g., hair disorders, nails, rosacea, perioral dermatitis, follicular syndromes, etc.);
Epidermal and appendageal tumors (e.g., squamous cell carcinoma, basal cell carcinoma, keratoacanthoma, benign epithelial tumors, merkel cell carcinoma, etc.);
Disorders of melanocytes (e.g., pigmentary disorders, albinism, hypomelanoses and hypermelanoses, melanocytic nevi, melanoma, etc.);
Skin disorders for inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, Kaposi sarcoma, etc.);
Disorders of the subcutaneous tissue (e.g., panninculitis, lipodystrophy, etc.);
Skin disorders involving cutaneous changes of altered reactivity (e.g., urticaria, angiodererma, graft-vs-host, allergic contact dermatitis, autosensitization dermatitis, atopic dermatitis, seborrheic dermatitis, etc.);
Skin changes due to mechanical and physical factors (e.g., thermal injury, radiation dermatitis, corns, calluses, etc.);
Photodamage (e.g., acute and chronic UV radiation, photosensitization, etc);
Skin disease due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, prions, etc.).

Targets of Skin Disease.

Proteins involved in mediating skin disease have been identified and thus can be targeted in a method to treat the skin disease (e.g., via topical targeting with an aptamer). Inflammatory dermatological disorders (e.g., atopic dermatitis, psoriasis, acne, etc.) include but are not limited to targets of cytokines and/or receptors such as IL2, IL12, IL17, IL22, IL23, IFNγ, TNFα, TSLP, etc. or chemokines CCR1, CXCR4, CRTH2. Proliferation disorders in the skin include (e.g., melanoma and non-melanoma skin cancers) include but are not limited to targets of TLRs, p53, RAS, MEK, PATCH1, mediators of sonic hedgehog signaling, etc. Fibroblast disorders in the skin (e.g., scarring after a trauma or surgery) or fibrosis, scleredoma, scar formation, (e.g., a keloid scar or a hypertrophic scar (miRNA (e.g., mir21), PI3K/AKT, PTEN)). Overproduction of sebum lipids in the skin (e.g., acne, etc) include but are not limited to targets of FAS, DGAT, SCD1, etc. Targets in chronic blistering (bullous) disorder, targets in cell growth, proliferation, and differentiation (EGFR, EGF, HB-EGF, TGF-α, EPR, BTC, NRG1-4, etc.) or adhesion (β-integrins, CD44, E-cadherin, CLA, etc.). Targets in infectious disease (HPV1, HPV5, HPV8, HPV14, HPV16, HPV17, HPV20, HPV31, HPV47, etc.). Targets in photo-damage and photo-ageing. Targets for vasculature and angiogenesis (VEGFC, VEGFD, VEGF, VEGF-R, etc.). Targets involved in wound repair or regeneration (EGF, FGF, GMCSF, IL-1, PDGF, TGF-β1, and their receptors, e.g., EGF-R, FGF-R, etc.).

Individual targets and/or their targets can include interactions with other biologic or chemical entities can be subject to modulation (including agonism, antagonism, stabilization, destabilization, enhanced clearing from cellular, organ, or whole body systems by aptamers) including soluble ligands and or their receptors, extracellular, transmembrane, and intracellular enzymes, scaffolding/structural proteins, RNA, DNA, lipids, fatty acids, co-factors and the respective specific and/or non-specific interactions.

Aptamers against these targets (e.g., proteins involved in mediating a skin disease) may be used to treat a skin disease, e.g., topically administered, e.g., as described herein.

Inflammatory Skin Disease

Topical formulations and/or topical administration of an IL-23 aptamer may be used for treating an inflammatory skin disease. Inflammatory skin disease refers to a disease that involves a series of clinical signs and symptoms, such as itching, edema, erythema and stripping, due to various stimulating factors which cause a series of inflammatory reactions in the skin. Known inflammatory skin diseases include, but are not limited to, dermatitis (e.g., atopic dermatitis, contact dermatitis, eczematous dermatitis, or seborrhoic dermatitis), acne, psoriasis, rosacea, or aging skin.

Inflammatory skin diseases are a common problem in dermatology. They come in many forms, from occasional rashes accompanied by skin itching and redness, to chronic conditions such as dermatitis, rosacea, and psoriasis. Skin inflammation can be characterized as acute or chronic. Acute inflammation can result from exposure to UV radiation (UVR), ionizing radiation, allergens, or to contact with chemical irritants (soaps, hair dyes, etc.). This type of inflammation is typically resolved within 1 to 2 weeks with little accompanying tissue destruction. Chronic inflammation results from a sustained immune cell mediated inflammatory response within the skin itself. This inflammation is long lasting and can cause significant and serious tissue destruction.

Chronic Inflammatory Skin Disease

Chronic inflammatory skin diseases include dermatitis (e.g., atopic dermatitis, contact dermatitis, eczematous dermatitis, and seborrhoic dermatitis), acne, psoriasis, rosacea, and aging skin.

Psoriasis.

Psoriasis is a chronic, autoimmune disease that appears on the skin.

People with psoriasis on less than three percent of their body are considered to have a mild case. Those with three to 10 percent of the body affected by psoriasis are considered a moderate case. More than 10 percent is considered severe.

Atopic Dermatitis.

Atopic dermatitis is a long-term (chronic) skin disorder that involves scaly and itchy rashes.

Chronic Inflammatory Skin Disease Treatments:

Topical Treatment:

Topical treatments for a chronic inflammatory skin disease (e.g., psoriasis or atopic dermatitis) include: salicyclic acid; coal tar; aloe vera; jojoba; zinc pyrithione; capsaicin; a keratolytic (e.g., contains an active ingredient of salicylic acid, lactic acid, urea, or phenol); calamine; camphor; diphenhydramine hydrochloride (HCl); benzocaine and menthol; a vitamin D analog such as calcipotriene (e.g., DOVONEX®) or calcitriol (e.g., VECTICAL® or ROCALTROL®); calcipotriene and betamethasone dipropionate (e.g., TACLONEX®); a topical retinoid such as tazarotene (e.g., TAZOREC® or AVAGE®); anthralin (e.g., ZITHRANOL™-RR); a corticosteroid, such as clobetasol propionate, betamethasone dipropionate, halobetasol propionate, fluocinonide, diflorasone diacetate, mometasone furoate, diflorasone diacetate, halcinonide, desoximetasone, fluticasone propionate, betamethasone valerate, flurandrenolide, triamcinolone acetonide, fluocinolone acetonide, hydrocortisone valerate, prednicarbate, desonide, hydrocortisone, alclometasone dipropionate, a calcineurin inhibitor such as tacrolimus (e.g., PROGRAF®) and/or pimecrolimus (e.g., ELIDEL®). For the treatment of a chronic inflammatory skin disease (e.g., psoriasis or atopic dermatitis), an IL-23 aptamer described herein can be used in combination with one or more topical treatments.

Phototherapy:

Phototherapies include: ultraviolet light B (UVB) phototherapy, such as narrow band UVB therapy; ultraviolet light A (UVA) phototherapy; sunlight; photochemotherapy, such as PUVA (psoralen and UVA); and/or Goeckerman therapy (combined UVB treatment and coal tar treatment). For the treatment of a chronic inflammatory skin disease (e.g., psoriasis or atopic dermatitis), an IL-23 aptamer described herein can be used in combination with one or more phototherapy.

Laser Treatment:

Laser treatments include: excimer laser and/or pulsed dye laser. For the treatment of a chronic inflammatory skin disease (e.g., psoriasis or atopic dermatitis), an IL-23 aptamer described herein can be used in combination with one or more laser treatment.

Non-Biologic Systemic Treatment:

Systemic treatments a chronic inflammatory skin disease (e.g., psoriasis or atopic dermatitis) include: a retinoid, such as acitretin (e.g., SORIATANE® or NEOTIGASON®); cyclosporine; methotrexate; hydroxyurea (e.g., HYDREA®); isotretinoin; mycophenolate mofetil; sulfasalazine; and/or 6-thioguanine. For the treatment of a chronic inflammatory skin disease (e.g., psoriasis or atopic dermatitis), an IL-23 aptamer described herein can be used in combination with one or more non-biologic systemic treatment.

Biologic Systemic Treatment:

Biologics are a class of systemic treatment. Biologic treatments for a chronic inflammatory skin disease (e.g., psoriasis or atopic dermatitis) include: alefacept (e.g., AMEVIVE®); efalizumab (e.g., RAPTIVA®); Tumor Necrosis Factor-alpha (TNF-a) blocker, such as etanercept (e.g., ENBREL®), adalimumab (e.g., HUMIRA®), infliximab (e.g., REMICADE®), or golimumab (e.g., SIMPONI®); and/or ustekinumab (e.g., STELARA®). For the treatment of a chronic inflammatory skin disease (e.g., psoriasis or atopic dermatitis), an IL-23 aptamer described herein can be used in combination with one or more biologic systemic treatment.

Combination Therapy

The term “combination” refers to the use of the two or more agents or therapies to treat the same subject, wherein the use or action of the agents or therapies overlap in time. The agents or therapies can be administered at the same time (e.g., as a single formulation that is administered to a subject or as two separate formulations administered concurrently) or sequentially in any order. Sequential administrations are administrations that are given at different times. The time between administration of the one agent and another agent can be minutes, hours, days, or weeks. An IL-23 aptamer can also be used to reduce the dosage of another therapy, e.g., to reduce the side-effects associated with another agent that is being administered (and vice versa). Accordingly, a combination can include administering a second agent at a dosage at least 10, 20, 30, or 50% lower than would be used in the absence of an IL-23 aptamer (and vice versa).

IL-23, IL-12, and Autoimmune Disease

The interleukin (IL)-12 family of cytokines, of which IL-23 is a member, is a group of structurally related proteins that are produced by antigen-presenting cells, including macrophages and dendritic cells, and that activate T-cells and Natural Killer (NK) cells [18-19]. The founding member, IL-12, is a heterodimeric protein consisting of the p40 and p35 subunits. IL-12 stimulates the differentiation of naïve T-cells into Th1 cells that produce IFN-γ (IFN-g) and other Th1 cytokines and further stimulates Th1 cells to proliferate [20-21].

IL-23 is a relatively new member of the IL-12 family that consists of the p40 subunit of IL-12 and a unique p19 subunit. IL-23 binds to Th17 and NK cells via a receptor distinct from the IL-12 receptor. The IL-23/IL-23R interaction has distinct effects, including the stimulation of Th17 cells to proliferate and to produce IL-17 and IL-22]22-25]. Studies using mice lacking only IL-23 (p19−/−), only IL-12 (p35−/−), or both cytokines (p40−/−) indicate that IL-23 rather than IL-12 is the more relevant cytokine for mouse EAE, mouse CIA, and H. hepaticus-induced T cell-dependent colitis in mouse models for MS, RA, and IBD respectively [26-29]. Furthermore, Chen et al. [30] reported that an anti-mouse p19 inhibitory antibody prevented mouse EAE induction and was able to reverse established disease.

IL-23 also differs from IL-12 in that its site of action is thought to be mainly in local tissue (i.e., skin, CNS, lung, etc.) rather than systemic (i.e., blood, lymph nodes). Several lines of evidence support this hypothesis (reviewed in 31). First, IL-23 is not involved in the genesis of circulating Th17 cells but instead stimulates the activation and proliferation of existing Th17 cells [31]. Thus, in some cases, deletion of IL-23 has not led to a significant decrease in circulating Th17 cells. Consistent with this, IL-23 is generally upregulated at sites of inflammation resulting in significantly more IL-23R expressing T-cells in lesions whereas levels of IL-23R expressing T-cells in circulation remain normal [32]. Second, exogenous IL-23 can rescue EAE in IL-23 knock-out mice when an expressing vector is injected into the brain but not when introduced into the blood [26]. Third, using IL-23R−/− instead of wild type (wt) CD4+ T-cells in the Rag1−/− colitis mouse model strongly inhibits colonic inflammation without affecting systemic inflammation [33].

Human IL-23 and IL-12 are both heterodimers that have one subunit in common and one unique subunit. The subunit in common is the p40 subunit which contains the following amino acid sequence (Accession #AF180563) (SEQ ID NO:3):

MCHQQLVISWFSLVFLASPLVAIWELKKDVYVVELDWYPDAPGE
MVVLTCDTPEEDGITWTLDQSSEVLGSGKTLTIQVKEFGDAGQY
TCHKGGEVLSHSLLLLHKKEDGIWSTDILKDQKEPKNKTFLRCE
AKNYSGRETCWWLTTISTDLTESVKSSRGSSDPQGVTCGAATLS
AERVRGDNKEYEYSVECQEDSACPAAEESLPIEVMVDAVHKLKY
ENYTSSFFIRDIIKPDPPKNLQLKPLKNSRQVEVSWEYPDTWST
PHSYFSLTFCVQVQGKSKREKKDRVFTDKTSATVICRKNASISV
RAQDRYYSSSWSEWASVPCS

The p19 subunit is unique to IL-23 and contains the following amino acid sequence (Accession #BC067511) (SEQ ID NO:4):

MLGSRAVMLLLLLPWTAQGRAVPGGSSPAWTQCQQLSQKLCTLA
WSAHPLVGHMDLREEGDEETTNDVPHIQCGDGCDPQGLRDNSQF
CLQRIHQGLIFYEKLLGSDIFTGEPSLLPDSPVGQLHASLLGLS
QLLQPEGHHWETQQIPSLSPSQPWQRLLLRFKILRSLQAFVAVA
ARVFAHGAATLSP

IL-23 and Inflammatory Skin Disease

IL-23 expression in the skin has been linked to chronic inflammatory skin diseases (e.g., psoriasis and atopic dermatitis) (Lowes, et al., Trends Immunol. Epublished: pii: S1471-4906(12)00199-8. doi: 10.1016/j.it.2012.11.005 (2013), “The IL-23/T17 pathogenic axis in psoriasis is amplified by keratinocyte responses” and Toussirot, Inflamm Allergy Drug Targets 11(2):159-68(2012)). Genetic association studies link certain gene variants located in the p19 subunit (gene IL-23a) and the specific subunit of the IL-23 receptor (gene IL-23R) to psoriasis, although the functional consequences of these variations have yet to be elucidated [34].

Many cell types in the skin either produce or respond to IL-23. Dendritic cells in the upper dermis and Langerhans cells plus keratinocytes in the epidermis produce IL-23 in psoriatic lesions but not normal skin in psoriasis patients (reviewed in 35). CD4+ T-cells in the dermis and CD8+ T-cells, keratinocytes, and neutrophils in the epidermis respond to IL-23, IL-17, and IL-22 in psoriatic lesions [36]. Not only is IL-23 present in the lesions of psoriatic patients, but direct injections or over-expression of IL-23 in the skin of mice also demonstrated that it is sufficient for the development of psoriasis-like lesions [37]. Lesion formation correlated with increased expression of mouse IL-17a, IL-23, and IL-22. Blockade of injected IL-23 with a systemically delivered anti-IL-23 monoclonal antibody (Mab) inhibited the formation of these lesions. Finally, injection of anti-IL-23 Mab can inhibit the development of inflammatory lesions in the mouse xenotransplant model of psoriasis [38].

Aptamers

Aptamers are synthetically derived nucleic acid molecules having specific binding affinity to molecules through interactions other than classic Watson-Crick base pairing. Aptamers are capable of specifically binding to selected targets and modulating the target's activity. Created by an in vitro selection process from pools of random sequence oligonucleotides, aptamers have been generated for over 100 proteins including growth factors, transcription factors, enzymes, immunoglobulins, and receptors (see, e.g., U.S. Pat. Nos. 5,270,163 and 5,843,653, and Bouchard et al., Annu. Rev. Pharmacol. Toxicol. (2010) 50:237-257). A typical aptamer may bind its target with sub-nanomolar affinity and discriminate against closely related targets.

Aptamers have a number of desirable characteristics for use as therapeutics and diagnostics including high specificity and affinity, biological efficacy, and excellent pharmacokinetic properties.

Aptamers are larger in size than antisense oligonucleotides. Antisense oligonucleotides are often about 20 nucleotides in length; in contrast, aptamers are generally larger, e.g, at least 30 nucleotides in length, and often larger, e.g., 50-90 nucleotides.

Another major difference between antisense oligonucleotides and aptamers are the conformational structures and flexibility (Nomura, Y., et al., (2010) Nucleic Acids Res 38(21):7822-9). Structure in antisense molecules is actively avoided because it can have an impact on efficacy. One of the advantages of aptamers is that structure can be modified and still maintain activity.

Aptamers may include modifications including e.g., conjugation to lipophilic or high molecular weight compounds (e.g., PEG), incorporation of a CpG motif, incorporation of a capping moiety, incorporation of modified nucleotides, and incorporation of phosphorothioate in the phosphate backbone, as described further herein.

In one embodiment, an isolated and/or non-naturally occurring aptamer that binds to IL-23 (IL-23) is provided. In some embodiments, the isolated and/or non-naturally occurring aptamer has an IC50 against IL-23 of less than 100 nM, less than 10 nM, less than 5 nM, less than 1 nM, less than 0.5 nM, less than 0.25 nM, or less than 0.1 nM. In some embodiments, the isolated and/or non-naturally occurring aptamer has an IC50 against IL-23 of 0.3 nM. In some embodiments of the invention, the IC50 is determined in an in vitro assay that measures the ability of the aptamer to inhibit endogenous IL-23 activation of PHA/IL-2 activated T cells (from PHA/IL-2 treated PBMC) by measuring levels of phospho-STAT3 by ELISA, as described herein (“phospho-STAT3 assay”).

In one embodiment, the aptamer has essentially the same ability (e.g., at least about 75%. about 50%, about 40% about 30%, or about 20% as well) to bind to IL-23 as that of an aptamer of SEQ ID NO:1 or 2.

In another embodiment of the invention, the aptamer has the structure (e.g., similar secondary structure, e.g., preservation of some or all loops; and/or tertiary structure) as that of an aptamer of SEQ ID NO:1 or 2 and can to bind to IL-23.

In another embodiment of the invention, the aptamer that binds to IL-23 has the structure (e.g., similar secondary structure, e.g., preservation of some or all loops; and/or tertiary structure) as that of an aptamer of SEQ ID NO:1 or 2 and can to inhibit (e.g., decrease) Stat3 phosphorylation, e.g., in the phospho-STAT3 assay described herein.

In another embodiment, an aptamer (e.g., the aptamer that binds to IL-23) is used as an active ingredient in a pharmaceutical composition.

In another embodiment, an aptamer (e.g., the aptamer that binds to IL-23) or a composition comprising the aptamer of the invention is used to treat psoriasis.

In another embodiment, the aptamer that binds to IL-23 or a composition comprising the aptamer of the invention is used to treat atopic dermatitis.

An aptamer (e.g., the aptamer that binds to IL-23) can be synthesized using standard phosphoramidites chemistry (Beaucage, S. L. and R. P. Iyer. “Advances in the Synthesis of Oligonucleotides by the Phosphoramidite Approach.” Tetrahedron 48(12): 2223-2311 (1992)).

An aptamer (e.g., the aptamer that binds to IL-23) can be provided as a lyophilized preparation, e.g., which can be reconstituted in a solution or cream, e.g., for administration, e.g., topical administration.

Percent Identity:

The term “sequence identity” in the context of two or more nucleic acid sequences, refers to two or more sequences or subsequences that are the same or have a specified percentage of nucleotides that are the same, when compared and aligned for maximum correspondence, as measured using one of the following sequence comparison algorithms or by visual inspection. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al.).

One example of an algorithm that is suitable for determining percent sequence identity is the algorithm used in the basic local alignment search tool (hereinafter “BLAST”), see, e.g., Altschul et al., J. Mol. Biol. 215: 403-410 (1990) and Altschul et al., Nucleic Acids Res., 15: 3389-3402 (1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) are described in McGinnis et al., Nucleic Acids Res., 32: W20-W25 (2004).

A Topical Anti-IL-23 Aptamer for Inflammatory Skin Disease

A topical, locally applied IL-23 inhibitor may be a useful approach as a therapy for an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)). Such an inhibitor could be as effective, e.g., as a p40 systemic inhibitor without systemic immune suppression. For example, such an inhibitor could expand the range of treatable psoriasis patients to include the much larger population of subjects with mild psoriasis.

An aptamer consisting of or containing the 62-mer oligonucleotide sequence (SEQ ID NO:1), such as ARC32225, can bind to and inhibit IL-23, and can be used, e.g., to treat an inflammatory skin disease (e.g., a chronic inflammatory skin disease (e.g., psoriasis or atopic dermatitis)) (e.g., can be topically applied to treat an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis))):

ARC32225 is the 62-mer oligonucleotide having the following sequence (SEQ ID NO:1):

5′-mA-fG-fG-fG-mA-mA-mA-fU-fC-fA-mG-fG-fC-
fU-fU-fU-mA-fU-fC-mG-mG-mG-fC-fG-fC-fC-mG-
fC-fU-fC-fC-fC-fU-mG-mU-mG-mC-fC-mA-fU-fC-
mG-fU-mC-mC-fG-mA-mG-mA-mG-fU-mA-mG-mG-fU-
mA-mG-fU-fC-fU-mG-idT-3′

The 2′ position of the sugar for each base is modified either by a 2′Fluoro or a 2′-O-methoxyethyl (2′OME) group. The 3′-inverted thymidine residue is added to increase nuclease resistance. One or more nucleotides (e.g., on the base or sugar) of an aptamer containing SEQ ID NO: ______ can be modified as described herein. An aptamer consisting of or containing the 62-mer oligonucleotide sequence (SEQ ID NO:1) can bind to and inhibit IL-23.

An aptamer consisting of or containing the 61-mer oligonucleotide of SEQ ID NO:2 can bind to and inhibit IL-23, and can be used, e.g., to treat an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)) (e.g., can be topically applied to treat an inflammatory skin disease (e.g., a chronic inflammatory skin disease (e.g., psoriasis or atopic dermatitis))):

5′-mA-fG-fG-fG-mA-mA-mA-fU-fC-fA-mG-fG-fC-
fU-fU-fU-mA-fU-fC-mG-mG-mG-fC-fG-fC-fC-mG-
fC-fU-fC-fC-fC-fU-mG-mU-mG-mC-fC-mA-fU-fC-
mG-fU-mC-mC-fG-mA-mG-mA-mG-fU-mA-mG-mG-fU-
mA-mG-fU-fC-fU-mG-3′

One or more nucleotides (e.g., on the base or sugar) of an aptamer containing SEQ ID NO:2 can be modified as described herein.

The aptamer of SEQ ID NO:1 or 2 can be stabilized by one or more methods described herein.

Dosing

Percent Applied Dose:

Topical formulations are applied to the skin in small amounts or a thin layer, referred to as finite dosing, which is thought to mimic the in vivo clinical situation. These amounts are referred to in amount (weight) of the vehicle (e.g., pharmaceutically acceptable composition) divided by surface area to describe surface area coverage. The FDA guidance is 2 mg/cm² or 2 μL/cm² which is based on what is required to for a homogenous film of defined thickness of 20 μm (FDA, Sunscreen drug products for over-the-counter human use, Fed Reg 43; pages 38206-38269, (1978)). Other citations suggest the range depends on the formulation or vehicle and its viscosity (Table 1) and the disease state. For skin diseases involving larger body surface area, Surber and Davis review the field related to the dose applied with topical formulations. They show that the amount applied vary from 0.7 to 4 mg/cm² (Surber and Davis, Bioavailability and bioequivalence, in Walters, K. A. (Ed.), Dermatological and Transdermal Formulations, Marcel Dekker, New York, Basel, pp. 401-498 (2002)). These amounts may vary in the clinical situation and could conceivably be applied at higher amounts of up to 20 mg/cm².

In vitro experiments use doses that range from 2-20 mg/cm² for finite dosing, with the standard dosing averaging around 10 mg/cm². This dosing volume can be used to estimate how much of the compound penetrates the skin as a percent of applied dose. For example, if a 1% aptamer formulation (10 mg/mL) was applied topically to ex vivo skin at a dose of 10 mg/cm² the total amount of active pharmaceutical ingredient (“API”) or in this instance, the aptamer or a composition comprising the aptamer applied to the surface of the skin is equal to 100 μg (assuming a 1.0 cm² dosing area). Concentrations in the skin could be converted to a percentage of this amount, where 1.0 μg would be equivalent to 1% of applied dose, 4.0 μg would be equivalent to 4% of applied dose, 10.0 μg would be equivalent to 10% of applied dose, etc. The concentrations in the epidermis have been shown to be typically above 1% applied dose and the concentrations in the dermis are above 0.1%. These levels are surprising and significant, as they are levels that are often observed with small molecules. Given that aptamers are typically more potent than traditional API small molecules, show these tissue levels are achieving (reaching) therapeutic levels.

TABLE 1
Finite dosing volumes used in clinical (in vivo) situations (from
Griffiths and Wilkinson, Topical Therapy, in: Rook A, Wilkinson
DS, et. al. eds. Textbook of Dermatology, London: Blackwell
Scientific, pages 3037-3084 (1992)).

| Site | To use sparingly | To use liberally | Lotions |
|---|---|---|---|
| Whole | 100 g | 250-500 g | 500 mL |
| Body | (~0.8 mg/cm², day) | (~2-4 mg/cm², day) | (~4 mg/cm², day) |
| Localized | 15-30 g | 50-100 g | 25-100 mL |
| Disease | | | |

Using the methods described herein, between 0.01% and 50% or between 0.1% and 50% (between 0.1% and 30%, between 0.1% and 20%, between 0.1% and 15%, between 0.1% and 10%, or between 0.1% and 5%) of the applied dose of a topically applied aptamer enters (permeates) into the epidermis (e.g., viable epidermis).

Using the methods described herein, at least 0.01%, at least 0.05%, at least 0.1%, at least 0.5%, at least 1%, at least 5%, at least 10%, at least 15%, at least 20%, at least 30%, at least 40% or at least 50% of the applied dose of a topically applied aptamer enters (permeates) into the epidermis (e.g., viable epidermis).

Using the methods described herein, between 0.01% and 50% (between 0.1% and 40%, between 0.1% and 30%, between 0.1% and 20%, between 0.1% and 15%, between 0.1% and 10%, or between 0.1% and 10%) of the applied dose of a topically applied aptamer enters (permeates) into the dermis.

Using the methods described herein, at least 0.01%, at least 0.05%, at least 0.1%, at least 0.5%, at least 1%, at least 5%, or at least 10% of the applied dose of a topically applied aptamer enters (permeates) into the dermis.

Pharmacodynamic Response.

In accordance with the methods described herein, delivery of aptamers at levels to achieve a pharmacodynamic (PD) response, i.e., the association of the aptamer with the biological target to evoke a physiological response, is possible. This can be related to the aptamer's affinity or intrinsic efficacy/activity. The intrinsic efficacy/activity can be measured quantitatively through surrogate systems for prediction of activity in therapeutic situations and is often presented as IC50s, EC50s, pEC50s, etc. depending on the pharmacological target (i.e., agonist vs. antagonist, etc.). Calculations of intrinsic efficacy/activity are routine in the field: This is a standard approach in drug discovery and industry during development (Terry Kenakin. “Pharmacodynamics in Pharmacology”, in Pharmacology vol. 1, pages 319-354, Harry Majewski, editor; Encyclopedia of Life Support Systems, UNESCO (2009)). As demonstrated herein, aptamers topically applied in accordance with the methods described herein achieve levels—in both the epidermis (e.g., viable epidermis) and dermis—that are in vast excess of the IC50 for the aptamer. Indeed, as shown herein, for an exemplified aptamer, aptamer levels greater than 100-, 500-, 1,000-, 10,000-, and even 100,000-fold above the IC50 can be attained in the epidermis (e.g., viable epidermis); and aptamer levels greater than 10-, 50-, 100-, 500-, 1,000- and even 5,000-fold above the IC50 can be attained in the dermis. In one embodiment of the invention, the topical application of an aptamer composition to the skin of a subject (e.g., human subject) comprises administering a dose of the aptamer composition to the subject, wherein the aptamer composition elicits an intrinsic activity in the epidermis or the dermis of the skin of the subject. In one embodiment, the response is in the epidermis. In another embodiment, it is in the dermis. In one embodiment, the activity is measured as an EC50 value (e.g., against a target of interest). In another embodiment, the activity is measured as an IC50 value (e.g., against a target of interest).

As used herein, intrinsic activity is synomous with pharmacodynamic response.

Pharmaceutical Compositions

The disclosure provides pharmaceutical compositions (e.g., pharmaceutically acceptable compositions) containing an aptamer. In some embodiments, the compositions are suitable for topical use and include, e.g., an effective amount of the aptamer, alone or in combination, with one or more vehicles (e.g., pharmaceutically acceptable compositions or e.g., pharmaceutically acceptable carriers).

The disclosure provides pharmaceutical compositions for topical administration containing an aptamer that binds to a target involved in mediating a skin disease. In some embodiments, the compositions for topical use can include, e.g., an effective amount of the aptamer, alone or in combination, with one or more vehicles (e.g., pharmaceutically acceptable carriers)

The compositions containing an aptamer can be used (e.g., by topical administration) to treat or prevent a skin disease. Treating a skin disease includes amelioration of at least one symptom of the skin disease.

The compositions containing an aptamer are useful for topical administration to a subject suffering from, or predisposed to, a skin disease. Skin diseases include: a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

The subject having a skin disease (e.g., a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions),i.e., the subject treated by a method of this disclosure, can be a vertebrate, more particularly a mammal, or more particularly a human.

The compositions containing an aptamer are useful for topical administration to a subject suffering from, or predisposed to, a skin disease. Skin diseases include: psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation. The compositions can be used in a method for treating a subject having an inflammatory skin disease (e.g., a chronic inflammatory skin disease (e.g., psoriasis or atopic dermatitis)). The method involves topically administering to the subject an aptamer or a composition comprising the aptamer, so that binding of the aptamer to its target decreases (e.g., inhibits) the biological function thereof, thereby treating a skin disease.

The subject having a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation), i.e., the subject treated by a method of this disclosure, can be a vertebrate, more particularly a mammal, or more particularly a human.

The aptamer can be topically administered in an amount (an effective amount) which will be sufficient to exert its desired biological activity in the skin.

The disclosure provides pharmaceutical compositions containing an aptamer (e.g., an aptamer containing the sequence of SEQ ID NO:1 or 2, such as ARC32225) that binds to IL-23. In some embodiments, the compositions are suitable for topical use and include, e.g., an effective amount of the aptamer, alone or in combination, with one or more vehicles (e.g., pharmaceutically acceptable carriers). In some embodiments, the compositions are suitable for internal use and include, e.g., an effective amount of the aptamer, alone or in combination, with one or more vehicles (e.g., pharmaceutically acceptable carriers).

The compositions containing an aptamer that binds to IL-23 can be used to treat or prevent a pathology associated with IL-23, e.g., an inflammatory skin disease (e.g., dermatitis (e.g., atopic dermatitis, contact dermatitis, eczematous dermatitis, or seborrhoic dermatitis), acne, psoriasis, rosacea, or aging skin, e.g., a chronic inflammatory skin disease (e.g., psoriasis or atopic dermatitis)).

The compositions containing an aptamer that binds to IL-23 are useful for administration (e.g., topical administration) to a subject suffering from, or predisposed to, an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)). The compositions can be used in a method for treating a subject having an inflammatory skin disease (e.g., a chronic inflammatory skin disease (e.g., psoriasis or atopic dermatitis)). The method involves administering to the subject an aptamer or a composition comprising the aptamer, so that binding of the aptamer to IL-23 decreases (e.g., inhibits) the biological function of IL-23, thereby treating an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)).

The subject having an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)), i.e., the subject treated by a method of this disclosure, can be a vertebrate, more particularly a mammal, or more particularly a human.

The aptamer that binds to IL-23, can be administered in an amount (an effective amount) which will be sufficient to exert its desired biological activity, e.g., inhibiting IL-23 function, e.g., assessed as described herein, e.g., by evaluating STAT3 phosphorylation, IL17 expression, or IL22 expression; and/or evaluated by its effects (e.g., amelioration of) of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)) (e.g., decrease in size or number of lesions, or, e.g., decrease in redness) or a symptom thereof.

Therapeutic or pharmacological compositions will generally include an effective amount of the active component(s) (e.g., an aptamer) of the therapy, dissolved or dispersed in a vehicle (e.g., pharmaceutically acceptable carrier or medium). Pharmaceutically acceptable media or carriers include solvents, dispersion media, thickeners, preservatives, coatings, antibacterial and antifungal agents, isotonic and absorption delaying agents and the like. Supplementary active ingredients can also be incorporated into the therapeutic compositions.

The preparation of pharmaceutical compositions will be known to those of skill in the art in light of the present disclosure. Such compositions may be prepared as topicals, injectables, either as liquid solutions or suspensions; solid forms suitable for solution in, or suspension in, liquid prior to injection; as time release capsules; or in any other form currently used, including eye drops, creams, lotions, salves, inhalants and the like. The use of sterile formulations may also be particularly useful. Compositions may also be delivered via microdevice, microparticle or sponge.

Upon formulation, the composition can be administered in a manner compatible with the dosage formulation (e.g., topically administered, e.g., as a cream), and in such amount (e.g., fixed concentration in a topical preparation) as is pharmacologically effective. The formulations are easily administered in a variety of dosage forms, such as topical preparations (e.g., creams), injectable solutions, and so forth.

In this context, the concentration or quantity of active ingredient and volume of composition to be administered depends on the host subject to be treated. Precise amounts of an aptamer required for administration may depend on the judgment of the practitioner.

A minimal volume of a composition required to disperse an aptamer can be utilized. Suitable regimes for administration can be variable, but may include initially administering a predetermined dose of an aptamer and monitoring the results and then giving a further controlled dose(s) at a further interval(s).

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to about 75%, preferably about 0.5% to about 50% or about 0.75% to about 10% (e.g., 1%) of the active ingredient.

Liquid, particularly injectable compositions can, for example, be prepared by dissolving, dispersing, etc. The aptamer can be dissolved in or mixed with a pharmaceutically pure solvent such as, for example, water, saline, aqueous dextrose, glycerol, ethanol, and the like, to thereby form the injectable solution or suspension. Additionally, solid forms (e.g., lyophilized forms) suitable for dissolving in liquid prior to injection can be formulated.

The aptamers of the present invention can be administered in intravenous (both bolus and infusion), intraperitoneal, subcutaneous, or intramuscular form, all using forms known to those of ordinary skill in the pharmaceutical arts. Injectables can be prepared in conventional forms, either as liquid solutions or suspensions.

Parenteral injectable administration is generally used for subcutaneous, intramuscular, or intravenous injections and infusions. Additionally, one approach for parenteral administration employs the implantation of a slow-release or sustained-released systems.

Furthermore, aptamers of the present invention can be administered in intranasal form via topical use of suitable intranasal vehicles, inhalants, or via transdermal routes, e.g., using those forms of transdermal skin patches known to those of ordinary skill in that art. To be administered in the form of a transdermal delivery system, the dosage administration can be continuous rather than intermittent. Other preferred topical preparations include creams, ointments, lotions, aerosol sprays and gels, wherein the concentration of active ingredient can range, e.g., from 0.01% to 25% (e.g., 1%), w/w or w/v or v/v.

For solid compositions, excipients include pharmaceutical grades of mannitol, lactose, starch, magnesium stearate, sodium saccharin, talcum, cellulose, glucose, sucrose, magnesium carbonate, and the like. The aptamer may be formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamers can be provided as a complex with a lipophilic compound or non-immunogenic, high molecular weight compound constructed using methods known in the art. An example of nucleic-acid associated complexes is provided in U.S. Pat. No. 6,011,020.

The aptamers may also be coupled with soluble polymers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate.

The dosage regimen utilizing the aptamers can be selected in accordance with a variety of factors including type, species, age, weight, sex, body surface area, and medical condition of the subject; the severity of the condition to be treated; the route of administration; the renal and hepatic function of the subject; and the particular aptamer or salt thereof employed.

Compositions of the present invention may be administered in a single daily dose, or the total daily dosage may be administered in divided doses, e.g., of two, three or four (etc.) times daily.

Topical Formulations

An aptamer can be formulated for topical administration. Suitable topical formulations of the aptamer include liquid, or semi-solid dosage forms (e.g., sprays (including aerosols), ointments, creams, gels, lotions, solutions, foams, and pastes). Formulations of the aptamer may also include liposomes, micelles, and/or microspheres. The aptamers may be also be in liquid, suspension, or powder form, so as in a suspension, or an emulsion, (including multiple emulsions), as a slow release nanoparticle, a slow release microparticle, a bioadhesive, a patch, a bandage or a wound dressing.

In some embodiments, the IL-23 aptamer can be formulated for topical administration. Suitable topical formulations of the IL-23 aptamer include liquid, or semi-solid dosage forms (e.g., sprays (including aerosols), ointments, creams, gels, lotions, solutions, foams, and pastes). Formulations of the IL-23 aptamer may also include liposomes, micelles, and/or microspheres. The IL-23 aptamers may be also be in liquid, suspension, or powder form, so as in a suspension, or an emulsion, (including multiple emulsions), as a slow release nanoparticle, a slow release microparticle, a bioadhesive, a patch, a bandage or a wound dressing.

The aptamer for topical administration can be formulated in a vehicle such as a spray (e.g., an aerosol), a liquid, an ointment, a cream, a lotion, a solution, a suspension, an emulsion, a paste, a gel, a powder, a foam, a slow release nanoparticle, a slow release microparticle, a bioadhesive, a patch, a bandage or a wound dressing.

The IL-23 aptamer for topical administration can be formulated in a vehicle such as a spray (e.g., an aerosol), a liquid, an ointment, a cream, a lotion, a solution, a suspension, an emulsion, a paste, a gel, a powder, a foam, a slow release nanoparticle, a slow release microparticle, a bioadhesive, a patch, a bandage or a wound dressing.

Ointments are semisolid preparations that are typically based on petrolatum or other petroleum derivatives. The specific ointment foundation to be used is one that will provide for optimum drug delivery, and, preferably, will provide for other desired characteristics as well, e.g., emolliency. As with other carriers or vehicles, the ointment foundation should be inert, stable, nonirritating and nonsensitizing. As explained in Remington: The Science and Practice of Pharmacy, 20th edition (Lippincott Williams & Wilkins, 2000), ointment foundations may be grouped in four classes: oleaginous, emulsifiable, emulsion, and water-soluble. Oleaginous ointment foundations include, for example, vegetable oils, fats obtained from animals, and semisolid hydrocarbons obtained from petroleum. Emulsifiable ointment foundations, also known as absorbent ointment foundations, contain little or no water and include, for example, hydroxystearin sulfate, anhydrous lanolin and hydrophilic petrolatum. Emulsion ointment foundations are either water-in-oil (W/O) emulsions or oil-in-water (O/W) emulsions, and include, for example, cetyl alcohol, glyceryl monostearate, lanolin and stearic acid. Some water-soluble ointment foundations are prepared from polyethylene glycols of varying molecular weight.

Creams are viscous liquids or semisolid emulsions, either oil-in-water (O/W) or water-in-oil (W/O). Cream foundations tend to be water-washable, and contain an oil phase, an emulsifier and an aqueous phase.

The oil phase, also called the “internal” phase, may be comprised of, but not limited to, one or more oils and/or fats. Exemplary oils and fats include, but are not limited to, fatty acids, esters, esters of glycerin, fatty alcohols, waxes, sterols, siloxanes and silanes, lanolin, hydrocarbons, essential oils, vegetable oils, mineral oils and edible oils, and mixtures thereof.

In one embodiment the oil is at least one of petrolatum and/or a fatty alcohol such as cetyl, or stearyl alcohol, or cetylstearoyl alcohol. The water or aqueous phase usually, although not necessarily, exceeds the oil phase in volume, and may contain at least one emulsifier or surfactant, The emulsifier in an oil/water or can be a nonionic, anionic, cationic or amphoteric surfactant.

A surfactant's hydrophilic/lipophilic balance (HLB) describes the surfactant's affinity toward water or oil. The HLB scale ranges from 1 (totally lipophilic) to 20 (totally hydrophilic), with 10 representing an equal balance of both characteristics. Lipophilic surfactants tend to form water-in-oil (w/o) emulsions, and hydrophilic surfactants tend to form oil-in-water (o/w) emulsions. The HLB of a blend of two surfactants equals the weight fraction of surfactant A times its HLB value plus the weight fraction of surfactant B times its HLB value (weighted average).

The formulations may include additional pharmaceutically acceptable excipients, such as a co-solvent, a preservative, an antioxidant, a pH adjusting agent, a chelating agent, and a penetration enhancer.

One non-limiting example of a suitable cream formulation can include one or more of: hydroxypropyl methylcellulose; isopropyl myristate; glyceryl monostearate; polyoxyl 40 stearate; glyceryl monostearate; methylparaben; and propylparaben.

Gels are semisolid, suspension-type systems. Single-phase gels contain organic macromolecules distributed substantially uniformly throughout the carrier liquid, which is typically aqueous, but also, preferably, contain an alcohol and, optionally, an oil (such as that described above). Suitable gelling agents include but are not limited to crosslinked acrylic acid polymers such as the “carbomer” family of polymers, e.g., carboxypolyalkylenes that may be obtained commercially as CARBOPOL®. Others gelling agents (also referred to as viscosity agents) are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions are preparations to be applied to the skin surface without friction, and are typically liquid or semiliquid preparations in which solid particles, including the active agent, are present in a water or alcohol base. Lotions are often suspensions of solids, and may comprise a liquid oily emulsion of the oil-in-water type. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Solutions are homogeneous mixtures prepared by dissolving one or more chemical substances (solute) in another liquid such that the molecules of the dissolved substance are dispersed among those of the solvent. The solution may contain other pharmaceutically acceptable chemicals to buffer, stabilize or preserve the solute. Commonly used examples of solvents or co-solvents used in preparing solutions include, but are not limited to, alcohols such as ethanol, isopropanol benzyl alcohol, water, diols, such as propylene or ethylene glycol and other polyols, such as glycerol.

Foams are defined as an emulsion containing one or more active ingredients, surfactants, aqueous or non-aqueous liquids, and the propellants (United States Pharmacopeia 32 (General Chapters: 1151). Foams can be summarized from several definitions as dynamic dosage forms intended for application to the skin; usually containing active agents, propellant, surface active agents, solvents and other excipients; prior to dose application they are sealed in a pressurized canister in the form of emulsion or suspension or solution; post dose or valve actuation, the propellant evaporates from the pressurized system producing a liquid or semi-solid foam product that is expanded with air (Zhao et al. Dynamic Foams in Topical Drug Delivery. J. Pharm & Pharmacology, June 2010).

Pastes are semisolid dosage forms in which the active agent is suspended in a suitable foundation. Depending on the nature of the foundation, pastes are divided between fatty pastes or those made from single-phase, aqueous gels. The foundation in a fatty paste is generally petrolatum or hydrophilic petrolatum or the like. The pastes made from single-phase aqueous gels generally incorporate carboxymethylcellulose or the like as the foundation.

Formulations may also be prepared with liposomes, micelles, and/or microspheres. Liposomes are microscopic vesicles having a lipid wall comprising a lipid bilayer, and can be used as drug delivery systems. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N-[1-2,3-dioleyloxy)propyl]-N,N,N-triethylammonium liposomes are available under the tradename LIPOFECTIN® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline, dioleoylphosphatidyl glycerol, and dioleoylphosphatidyl ethanolamine. These materials can also be mixed with N-[1-2,3-dioleyloxy)propyl]-N,N,N-triethylammonium (DOTMA) in appropriate ratios. Methods for making liposomes using these materials are known in the art.

Micelles are comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. For example, micelle formulations can be used by incorporation into the reservoir of a topical or transdermal delivery system, or into a formulation to be applied to the body surface.

Microspheres may be incorporated into formulations and drug delivery systems. Like liposomes and micelles, microspheres essentially encapsulate a drug or drug-containing formulation. They are generally, although not necessarily, formed from lipids, preferably charged lipids such as phospholipids. Preparation of lipidic microspheres is known in the art.

Various additives may be included in topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds.

The formulation may also contain irritation-mitigating additives to minimize or eliminate the possibility of skin irritation or skin damage resulting from the aptamer, the base enhancer, or other components of the formulation. Suitable irritation-mitigating additives include, for example: alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the formulation at a concentration effective to mitigate irritation or skin damage, typically representing not more than about 20 wt %, more typically not more than about 5 wt %, of the formulation.

The concentration of the active agent (e.g., aptamer) in the formulation will typically depend upon a variety of factors, including the disease or condition to be treated (e.g., an inflammatory skin disease, e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)), the nature and activity of the active agent (e.g., aptamer), the desired effect, possible adverse reactions, the ability and speed of the active agent (e.g., aptamer) to reach its intended target, body surface area, and other factors. Formulations may contain, for example, on the order of about 0.001-50 wt %, 0.5-50 wt %, or about 0.75-10% wt %, or e.g., about 1 w/w or v/v %, active agent (e.g., aptamer).

Exemplified Vehicles.

In the course of the studies presented herein, numerous vehicles for topical delivery (e.g., delivery to the surface of the skin, e.g., intact (e.g., uncompromised) skin) to a subject (e.g., to a mammalian subject, e.g., human) of an active ingredient, such as an aptamer (e.g., an IL-23 aptamer) or other oligonucleotide (e.g., antisense oligonucleotide), or small molecule, were developed. See, e.g., Tables 4 and 9, and e.g., wherein the listed IL23 aptamer is replaced with another active ingredient of interest.

In one embodiment of the invention, the aptamer composition does not contain a traditional surfactant. In another embodiment, the aptamer composition contains less than 2% of a surfactant, and in another embodiment less than 1% of a surfactant.

In one embodiment of the invention, the aptamer composition does not contain a penetration enhancer. In another embodiment, the aptamer composition contains less than 5% of a penetration enhancer, and in another embodiment less than 2% of a penetration enhancer.

In one embodiment, the present invention provides for a topical pharmaceutical composition comprising a therapeutically effective amount of an aptamer, an oil phase, a water phase, and a surfactant, wherein the composition is an oil-in-water cream.

The disclosure provides a vehicle for topical delivery of an active agent (e.g., an aptamer), wherein the vehicle comprises Caprylic/Capric Triglyceride, Glyceryl Monocaprylate, Polysorbate 80, Sorbitan Oleate, and Demineralized Water, e.g., about in the amounts listed in Table 4.

The disclosure provides a vehicle for topical delivery of an active agent (e.g., an aptamer), wherein the vehicle comprises Caprylic/Capric Triglyceride, Glyceryl Monocaprylate, Polysorbate 80, Sorbitan Oleate, Isopropanol, and Demineralized Water, e.g., about in the amounts listed in Table 4.

The disclosure provides a vehicle for topical delivery of an active agent (e.g., an aptamer), wherein the vehicle comprises Polysorbate 80, Demineralized Water, Propylene Glycol Monocaprylate, Propylene Glycol Dicaprylocaprate, and Diethyleneglycol Monoethyl Ether, e.g., about in the amounts listed in Table 4.

The disclosure provides a vehicle for topical delivery of an active agent (e.g., an aptamer), wherein the vehicle comprises Isopropanol, Demineralized Water, Benzyl Alcohol, Drakeol-5, Brij O2, Brij O10, and Cetrimonium chloride 30% solution, e.g., about in the amounts listed in Table 4.

The disclosure provides a vehicle for topical delivery of an active agent (e.g., an aptamer), wherein the vehicle comprises Demineralized Water, Benecel MP333C, Isopropyl Myristate, Glyceryl Monostearate NSE 400, Polyoxyl-40-stearate, and Phenonip, e.g., about in the amounts listed in Table 4.

The disclosure provides a vehicle for topical delivery of an active agent (e.g., an aptamer), wherein the vehicle comprises Deionized Water, Isopropyl Myristate, Glyceryl Monostearate NSE 400, Polyoxyl-40-stearate, Phenonip, and Benecel MP333C, e.g., about in the amounts listed in Table 9.

The disclosure provides a vehicle for topical delivery of an active agent (e.g., an aptamer), wherein the vehicle comprises Deionized Water, Xanthan Gum, Isopropyl Myristate, Glyceryl Monostearate NSE 400, Polyoxyl-40-stearate, Citric Acid Anhydrous, Potassium Citrate Monohydrate, Phenoxyethanol, and Benzyl Alcohol, e.g., about in the amounts listed in Table 9.

The disclosure provides a vehicle for topical delivery of an active agent (e.g., an aptamer), wherein the vehicle comprises Deionized Water, Isopropyl Myristate, Cithrol GMS 40 SE-PW-(SG), Polyoxyl-40-stearate, Benecel MP333C, Phenoxyethanol, and Benzyl Alcohol, e.g., about in the amounts listed in Table 9.

The disclosure provides a vehicle for topical delivery of an active agent (e.g., an aptamer), wherein the vehicle comprises Deionized Water, Cetostearyl Alcohol, White Petrolatum, Drakeol 9, Cetomacrogol 1000 BP, Tetrasodium EDTA, Disodium EDTA, Glycerin, Phenoxyethanol, and Benzyl Alcohol, e.g., about in the amounts listed in Table 9.

The disclosure provides a vehicle for topical delivery of an active agent (e.g., an aptamer), wherein the vehicle comprises Deionized Water, Isopropyl Myristate, Cetostearyl Alcohol, Drakeol 9, Cetomacrogol 1000 BP, Citric Acid Anhydrous, Potassium Citrate Monohydrate, Propylene Glycol, Phenoxyethanol, and Benzyl Alcohol, e.g., about in the amounts listed in Table 9.

The disclosure provides a vehicle for topical delivery of an active agent (e.g., an aptamer), wherein the vehicle comprises Deionized Water, Isopropyl Myristate, ST-5 Cyclomethicone, White Petrolatum, Drakeol 9, Cetomacrogol 1000 BP, Cetyl Alcohol, Sorbitan Monolaurate, Citric Acid Anhydrous, Potassium Citrate Monohydrate, Propylene Glycol, and Phenoxyethanol, e.g., about in the amounts listed in Table 9.

The disclosure provides a vehicle for topical delivery of an active agent (e.g., an aptamer), wherein the vehicle comprises Deionized Water, Isopropyl Myristate, ST-5 Cyclomethicone, Drakeol 9, Cetomacrogol 1000 BP, Cetyl Alcohol, Sorbitan Monolaurate, Citric Acid Anhydrous, Potassium Citrate Monohydrate, Propylene Glycol, and Phenoxyethanol, e.g., about in the amounts listed in Table 9.

The disclosure provides a vehicle for topical delivery of an active agent (e.g., an aptamer), wherein the vehicle comprises Deionized Water, DIPA, Isopropyl Myristate, Drakeol 9, Eumulgin B1 PH-Ceteareth 12, Citric Acid Anhydrous, Potassium Citrate Monohydrate, Propylene Glycol, and Benzyl Alcohol, e.g., about in the amounts listed in Table 9.

The disclosure provides a vehicle for topical delivery of an active agent (e.g., an aptamer), wherein the vehicle comprises Deionized Water, DIPA, Light Liquid Paraffin, Eumulgin B1 PH-Ceteareth 12, BHT, Citric Acid Anhydrous, Potassium Citrate Monohydrate, Sorbic Acid, and Potassium Sorbate, e.g., about in the amounts listed in Table 9.

The disclosure provides a vehicle for topical delivery of an active agent (e.g., an aptamer), wherein the vehicle comprises Deionized Water, Cetostearyl Alcohol, White Petrolatum, Drakeol 9, Cetomacrogol 1000 BP, Citric Acid Anhydrous, Potassium Citrate Monohydrate, Propylene Glycol, Phenoxyethanol, and Benzyl Alcohol, e.g., about in the amounts listed in Table 9.

The disclosure provides a vehicle for topical delivery of an active agent (e.g., an aptamer), wherein the vehicle comprises Deionized Water, Isopropyl Myristate, Propylene Glycol, Benzyl Alcohol, Transcutol P, and Dimethyl Isosorbide, e.g., about in the amounts listed in Table 9.

The disclosure provides a vehicle for topical delivery of an active agent (e.g., an aptamer), wherein the vehicle comprises Deionized Water, Polysorbate 20, and Benzyl Alcohol, e.g., about in the amounts listed in Table 9.

As used herein, “about” refers to amounts within 10% (e.g., 10% more or 10% less) of the amount listed in the table. Preferably, “about” refers to amounts within 5% more or 5% less of the amount recited. More preferably “about” refers to amounts within 2% more or 2% less of the amount recited.

The disclosure also provides a method of topically administering an active ingredient to a subject (e.g., a mammalian subject, e.g., a human) (e.g., to the surface of the skin, e.g., intact (e.g., uncompromised) skin), the method comprising:

topically administering to the subject a vehicle, e.g., a vehicle listed in Table 4 or 9 containing the components listed therein (excluding the listed IL-23 aptamer), e.g., wherein the components of the vehicle are in about the amounts listed therein, wherein the vehicle comprises the active ingredient.

In some embodiments, the active ingredient is an aptamer (e.g., an IL-23 aptamer).

In some embodiments of the method, the vehicle comprises Caprylic/Capric Triglyceride, Glyceryl Monocaprylate, Polysorbate 80, Sorbitan Oleate, and Demineralized Water, e.g., about in the amounts listed in Table 4.

In some embodiments of the method, the vehicle comprises Caprylic/Capric Triglyceride, Glyceryl Monocaprylate, Polysorbate 80, Sorbitan Oleate, Isopropanol, and Demineralized Water, e.g., about in the amounts listed in Table 4.

In some embodiments of the method, the vehicle comprises Polysorbate 80, Demineralized Water, Propylene Glycol Monocaprylate, Propylene Glycol Dicaprylocaprate, and Diethyleneglycol Monoethyl Ether, e.g., about in the amounts listed in Table 4.

In some embodiments of the method, the vehicle comprises Isopropanol, Demineralized Water, Benzyl Alcohol, Drakeol-5, Brij O2, Brij O10, and Cetrimonium chloride 30% solution, e.g., about in the amounts listed in Table 4.

In some embodiments of the method, the vehicle comprises Demineralized Water, Benecel MP333C, Isopropyl Myristate, Glyceryl Monostearate NSE 400, Polyoxyl-40-stearate, and Phenonip, e.g., about in the amounts listed in Table 4.

In some embodiments of the method, the vehicle comprises Deionized Water, Isopropyl Myristate, Glyceryl Monostearate NSE 400, Polyoxyl-40-stearate, Phenonip, and Benecel MP333C, e.g., about in the amounts listed in Table 9.

In some embodiments of the method, the vehicle comprises Deionized Water, Xanthan Gum, Isopropyl Myristate, Glyceryl Monostearate NSE 400, Polyoxyl-40-stearate, Citric Acid Anhydrous, Potassium Citrate Monohydrate, Phenoxyethanol, and Benzyl Alcohol, e.g., about in the amounts listed in Table 9.

In some embodiments of the method, the vehicle comprises Deionized Water, Isopropyl Myristate, Cithrol GMS 40 SE-PW-(SG), Polyoxyl-40-stearate, Benecel MP333C, Phenoxyethanol, and Benzyl Alcohol, e.g., about in the amounts listed in Table 9.

In some embodiments of the method, the vehicle comprises Deionized Water, Cetostearyl Alcohol, White Petrolatum, Drakeol 9, Cetomacrogol 1000 BP, Tetrasodium EDTA, Disodium EDTA, Glycerin, Phenoxyethanol, and Benzyl Alcohol, e.g., about in the amounts listed in Table 9.

In some embodiments of the method, the vehicle comprises Deionized Water, Isopropyl Myristate, Cetostearyl Alcohol, Drakeol 9, Cetomacrogol 1000 BP, Citric Acid Anhydrous, Potassium Citrate Monohydrate, Propylene Glycol, Phenoxyethanol, and Benzyl Alcohol, e.g., about in the amounts listed in Table 9.

In some embodiments of the method, the vehicle comprises Deionized Water, Isopropyl Myristate, ST-5 Cyclomethicone, White Petrolatum, Drakeol 9, Cetomacrogol 1000 BP, Cetyl Alcohol, Sorbitan Monolaurate, Citric Acid Anhydrous, Potassium Citrate Monohydrate, Propylene Glycol, and Phenoxyethanol, e.g., about in the amounts listed in Table 9.

In some embodiments of the method, the vehicle comprises Deionized Water, Isopropyl Myristate, ST-5 Cyclomethicone, Drakeol 9, Cetomacrogol 1000 BP, Cetyl Alcohol, Sorbitan Monolaurate, Citric Acid Anhydrous, Potassium Citrate Monohydrate, Propylene Glycol, and Phenoxyethanol, e.g., about in the amounts listed in Table 9.

In some embodiments of the method, the vehicle comprises Deionized Water, DIPA, Isopropyl Myristate, Drakeol 9, Eumulgin B1 PH-Ceteareth 12, Citric Acid Anhydrous, Potassium Citrate Monohydrate, Propylene Glycol, and Benzyl Alcohol, e.g., about in the amounts listed in Table 9.

In some embodiments of the method, the vehicle comprises Deionized Water, DIPA, Light Liquid Paraffin, Eumulgin B1 PH-Ceteareth 12, BHT, Citric Acid Anhydrous, Potassium Citrate Monohydrate, Sorbic Acid, and Potassium Sorbate, e.g., about in the amounts listed in Table 9.

In some embodiments of the method, the vehicle comprises Deionized Water, Cetostearyl Alcohol, White Petrolatum, Drakeol 9, Cetomacrogol 1000 BP, Citric Acid Anhydrous, Potassium Citrate Monohydrate, Propylene Glycol, Phenoxyethanol, and Benzyl Alcohol, e.g., about in the amounts listed in Table 9.

In some embodiments of the method, the vehicle comprises Deionized Water, Isopropyl Myristate, Propylene Glycol, Benzyl Alcohol, Transcutol P, and Dimethyl Isosorbide, e.g., about in the amounts listed in Table 9.

In some embodiments of the method, the vehicle comprises Deionized Water, Polysorbate 20, and Benzyl Alcohol, e.g., about in the amounts listed in Table 9.

In some embodiments of the method, the active ingredient is topically administered to treat a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, adult respiratory distress syndrome, pulmonary fibrosis, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation).

In some embodiments of the method, the active ingredient is topically administered to treat a skin disease, e.g., a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the medicament is for the treatment of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)).

The disclosure also provides a vehicle for topically administering an active ingredient to a subject (e.g., a mammalian subject, e.g., a human) (e.g., to the surface of the skin, e.g., intact (e.g., uncompromised) skin), wherein the vehicle is a vehicle listed in Table 4 or 9 containing the components listed therein (excluding the listed aptamer), e.g., wherein the components of the vehicle are in about the amounts listed therein, wherein the vehicle comprises the active ingredient.

In some embodiments, the active ingredient is an aptamer (e.g., an IL-23 aptamer).

In some embodiments, the vehicle is topically administered to treat a skin disease (e.g., a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the vehicle is topically administered to treat a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, pulmonary fibrosis, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation).

In some embodiments, the medicament is for the treatment of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)).

The disclosure also provides use of a vehicle for the preparation of a medicament for topical administration of an active ingredient to a subject (e.g., a mammalian subject, e.g., a human) (e.g., to the surface of the skin, e.g., intact (e.g., uncompromised) skin), wherein the vehicle is a vehicle listed in Table 4 or 9 containing the components listed therein (excluding the listed aptamer), e.g., wherein the components of the vehicle are in about the amounts listed therein, wherein the vehicle comprises the active ingredient.

In some embodiments, the active ingredient is an aptamer (e.g., an IL-23 aptamer). In some embodiments, the medicament is for the treatment of a skin disease (e.g., a skin disorder of persistent inflammation, cell kinetics, and differentiation (e.g., psoriasis, psoriatic arthritis, exfoliative dermatitis, pityriasis rosea, lichen planus, lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBV (Epstein-Barr virus), HPV (Human papillomavirus) (e.g., HPV6 &7), warts, or prions).

In some embodiments, the active ingredient is an aptamer (e.g., an IL-23 aptamer). In some embodiments, the medicament is for the treatment of a skin disease (e.g., psoriasis, dermatitis (e.g., atopic dermatitis, contact dermatitis, eczematous dermatitis, or seborrhoic dermatitis), a chronic blistering (bullous) disorder, acne, seborrhoeic cutaneous manifestations of immunologically-mediated disorders, alopecia, alopecia greata, scleredoma, scar formation, (e.g., a keloid scar or a hypertrophic scar), urticaria, rosacea, melanoma, exacerbations of chronic obstructive pulmonary disease (COPD), inflammation from kidney transplant, exacerbations of asthma, hidradentis supporativa, exacerbations of rheumatoid arthritis, exacerbations of psoriatic arthritis, exacerbations of Sjogren's Syndrome, exacerbations of uveitis, exacerbations of Graft vs. Host disease (GVHD), Oral Lichen Planus, exacerbations of arthralgia, or exacerbations of Islet Cell Transplant inflammation).

In some embodiments, the medicament is for the treatment of an inflammatory skin disease (e.g., a chronic inflammatory skin disease (e.g., dermatitis (e.g., atopic dermatitis, contact dermatitis, eczematous dermatitis, or seborrhoic dermatitis), acne, psoriasis, rosacea, or aging skin, e.g., psoriasis or atopic dermatitis)).

Modulation of Pharmacokinetics and Biodistribution of Aptamer Therapeutics

It is important that the pharmacokinetic properties for all oligonucleotide-based therapeutics, including aptamers, be tailored to match the desired pharmaceutical application. While aptamers directed against extracellular targets do not suffer from difficulties associated with intracellular delivery, such aptamers must still be able to be distributed to target organs and tissues, and remain at the site of treatment (e.g., diseased (e.g., psoriatic) skin) or in the body (unmodified) for a period of time consistent with the desired dosing regimen.

The present disclosure provides materials and methods to affect the pharmacokinetics of aptamer compositions, and, in particular, the ability to tune aptamer pharmacokinetics. The tunability of (i.e., the ability to modulate) aptamer pharmacokinetics can be achieved through conjugation of modifying moieties (e.g., PEG polymers) to the aptamer and/or the incorporation of modified nucleotides (e.g., 2′-fluoro or 2′-O-methyl, e.g., on the sugar groups) to alter the chemical composition of the nucleic acid. The ability to tune aptamer pharmacokinetics is used in the improvement of existing therapeutic applications, or alternatively, in the development of new therapeutic applications. For example, in some therapeutic applications, e.g., where the site of action is topically accessed (e.g., in the treatment of the skin, e.g., diseased (e.g., psoriatic skin)) or in anti-neoplastic or acute care settings or when sustained systemic distribution of the aptamer is not desired, where rapid drug clearance or turn-off may be desired, it is desirable to decrease the residence times of aptamers in the circulation. Alternatively, in other therapeutic applications, e.g., maintenance therapies where systemic circulation of a therapeutic is desired, it may be desirable to increase the residence times of aptamers in circulation.

In addition, the tunability of aptamer pharmacokinetics can be used to modify the biodistribution of an aptamer in a subject. For example, in some therapeutic applications, it may be desirable to alter the biodistribution of an aptamer therapeutic in an effort to target a particular type of tissue or a specific organ (or set of organs). In these applications, the aptamer preferentially accumulates in a specific tissue or organ(s). In other therapeutic applications, it may be desirable to target tissues displaying a cellular marker or a symptom associated with a given disease, cellular injury or other abnormal pathology, such that the aptamer therapeutic preferentially accumulates in the affected tissue.

To determine the pharmacokinetic and biodistribution profiles of aptamer therapeutics (e.g., aptamer conjugates or aptamers having altered chemistries, such as modified nucleotides) a variety of parameters can be monitored. Such parameters include, for example, the half-life (t½), the plasma clearance (C1), the volume of distribution (Vss), the area under the concentration-time curve (AUC), maximum observed serum or plasma concentration (Cₘₐₓ), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration) and/or total clearance (C1) (i.e., the rate at which the aptamer therapeutic is removed from circulation) of a given aptamer. The volume of distribution relates the plasma concentration of an aptamer therapeutic to the amount of aptamer present in the body. The larger the Vss, the more an aptamer is found outside of the plasma (i.e., the more extravasation).

The disclosure provides materials and methods to modulate, in a controlled manner, the pharmacokinetics and biodistribution of stabilized aptamer compositions in vivo, e.g., by conjugating an aptamer to a modulating moiety such as a small molecule, peptide, or polymer terminal group, or by incorporating modified nucleotides into an aptamer. As described herein, conjugation of a modifying moiety and/or altering nucleotide(s) chemical composition alters fundamental aspects of aptamer residence time in circulation and distribution to tissues.

In addition to clearance by nucleases, oligonucleotide therapeutics are subject to elimination via renal filtration. For example, a nuclease-resistant oligonucleotide administered intravenously may exhibit an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation of aptamers to a PEG (polyethylene glycol) polymer (PEGylation), described below, can dramatically lengthen residence times of aptamers in circulation, thereby decreasing dosing frequency.

Aptamers can be conjugated to a variety of modifying moieties, such as high molecular weight polymers, e.g., PEG; peptides, e.g., Tat (a 13-amino acid fragment of the HIV Tat protein (Vives, et al., (1997), J. Biol. Chem. 272(25): 16010-7)), Ant (a 16-amino acid sequence derived from the third helix of the Drosophila antennapedia homeotic protein (Pietersz, et al., (2001), Vaccine 19(11-12): 1397-405)) or Arg7 (a short, positively charged cell-permeating peptides composed of polyarginine (Arg.sub.7) (Rothbard, et al., (2000), Nat. Med. 6(11): 1253-7; Rothbard, J et al., (2002), J. Med. Chem. 45(17): 3612-8)); and small molecules, e.g., lipophilic compounds such as cholesterol. Among the various conjugates described herein, in vivo properties of aptamers are altered most profoundly by complexation with PEG groups. For example, complexation of a mixed 2′-F and 2′-OMe modified aptamer therapeutic with a 20 kDa PEG polymer can hinder renal filtration and promote aptamer distribution to both healthy and inflamed tissues. Furthermore, the 20 kDa PEG polymer-aptamer conjugate can prove nearly as effective as a 40 kDa PEG polymer in preventing renal filtration of aptamers. While one effect of PEGylation is on aptamer clearance, the prolonged systemic exposure afforded by presence of the 20 kDa moiety also facilitates distribution of an aptamer to tissues, particularly those of highly perfused organs.

Modified nucleotides can also be used to modulate the plasma clearance of aptamers. For example, an unconjugated aptamer which incorporates both 2′-F and 2′-OMe stabilizing chemistries, which is typical of current generation aptamers as it exhibits a high degree of nuclease stability in vitro and in vivo, may display rapid loss from plasma (i.e., rapid plasma clearance) and a rapid distribution into tissues when compared to unmodified aptamer.

2′-O-methyl, 2′-fluoro and other modified nucleotide modifications can stabilize an aptamer against nucleases and increase its half life in vivo. A 3′-idT cap can increase exonuclease resistance. See, e.g., U.S. Pat. Nos. 5,674,685; 5,668,264; 6,207,816; and 6,229,002.

PEG-Derivatized Nucleic Acids

As described above, derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamers of the invention may be derivatized with polyalkylene glycol (“PAG”) moieties. Typical polymers used in the invention include polyethylene glycol (“PEG”), also known as polyethylene oxide (“PEO”) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpolyethylene glycol, can also be represented as HO-PEG-OH, where it is understood that the -PEG- symbol represents the following structural unit: —CH₂CH₂O—(CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups can be activated or converted to functional moieties for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively non-reactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules.

Polyalkylated compounds of the invention are typically between 5 and 80 kDa in size however any size can be used, the choice dependent on the aptamer and application. For example, other PAG compounds can be between 10 and 80 kDa or 10 and 60 kDa in size. For example, a PAG polymer may be at least 10, 20, 30, 40, 50, 60, or 80 kDa in size. Such polymers can be linear or branched. In some embodiments, the polymers are PEG. In some embodiments, the polymers are branched PEG.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa.

One method for generating higher molecular weight activated PEGs involves the formation of a branched activated PEG in which two or more PEGs are attached to a central core carrying the activated group. The terminal portions of these higher molecular weight PEG molecules, i.e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol. 2: 214-221, 2003).

PEG-Derivatization of a Reactive Nucleic Acid

High molecular weight PEG-nucleic acid-PEG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In an embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully, partially, and un-conjugated species.

The linking domains can also have one or more polyalkylene glycol moieties attached thereto. Such PEGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with IL-23 will preclude the formation of a complex between the aptamer and IL-23. A linker, which is longer than necessary, may reduce binding stability by diminishing the effective concentration of the ligand. Thus, it may be necessary to optimize linker compositions and lengths in order to maximize the affinity of an aptamer to a target.

Topically Administered Aptamers as Biosensors

Aptamers have been selected against a broad range of targets such as metal ions, small molecules, peptides, proteins, and cells (Keefe et al., Nat. Rev. Drug Discov. (2010) 9:537-550).

Because of the high specificity and affinity that can be obtained for a target of interest, aptamers are well suited for detection of targets of interest (e.g., for diagnostic purposes). With these desirable characteristics, aptamer-based sensors have been developed to detect a diverse set of targets including adenosine, adenosine triphosphate (ATP), cocaine, Hg2+, toxins, thrombin, vascular endothelial growth factor (VEGF), IgE, and many others. See, e.g., Wang et al., Curr Med Chem (2011) 18(27):4175-4184.

As disclosed herein, topically applied aptamers are able to penetrate skin, even intact skin. Thus, topically applied aptamers can be valuable tools as biosensors for a target of interest that may be present in the skin (e.g., epidermis (e.g., viable epidermis) or dermis), e.g., for diagnostic purposes (e.g., for cancer (e.g., melanoma) cell detection, e.g., using an aptamer that binds to a cancer-specific target, e.g. using an aptamer to bind to a cancer cell). As a general matter, the method involves topically applying to the skin (e.g., intact skin) of a subject (e.g., human) an aptamer specific for a target of interest that may be present in the skin, and detecting the aptamer bound to the target (and e.g., either quantifying the amount of aptamer bound to its target or qualitatively tagging the target). The aptamer can be conjugated to a fluorescent label (fluorophore), numerous examples of which are known in the art, such as fluorescein-, bis-pyrenyl-, DMDAP-, ATMND-, and quantum dot-based fluorophores. The method of detection can be based on an increase in fluorescent signal upon aptamer binding to its target; or it can be based on a decrease in fluorescent signal upon aptamer binding to its target. More specifically, various methods can be employed to use topically-applied aptamers as biosensors for a target of interest that may be present in the skin (e.g., epidermis (e.g., viable epidermis) or dermis), as the following examples demonstrate. Changes in fluorescence can be detected and measured by methods such as fluorescent microscopy, MRI, PET, or fluorescent lifetime imaging microscopy (FLIM). In some methods, binding of aptamer to its target can be detected and measured using amplification methods such as PCR or DHA.

Aptamers are well known to have distinctly different conformations/structures before and after binding with the targets. Through direct modification with fluorophores, the conformational and/or structural changes of aptamers can affect the fluorescence of a dye. The signal change, either increase (i.e., the “signal-on” mode) or decrease (i.e. the “signal-off” mode), can reflect the extent of the binding process thereby allowing for qualitative or quantitative measurement of target concentration.

In a typical “signal-on” biosensor based on fluorescently-labeled aptamers, one end of the aptamer is conjugated to a fluorophore with its fluorescence signal quenched by a quencher (which can also be another fluorophore). Upon separation of the fluorophore and the quencher (through aptamer binding), the fluorescence signal is recovered which can be used for detection and qualitative or quantitative measurement of the target concentration.

In many cases, conformational change of an aptamer upon target binding can significantly affect the fluorescence of a conjugated dye.

Fluorophores can used to modify the 2′-ribose at selected positions of a number of aptamers, which exhibit increased fluorescence upon target binding of the aptamer (Merino et al., J. Am. Chem. Soc. (2005) 127:12766-12767). These biosensors can have wide detection ranges in buffer solutions and even in biological samples. Since fluorophore conjugation may reduce the target binding affinity of an aptamer, through either steric hindrance or interference with its folding, fluorescein-modified uridine can be incorporated into the nucleoside triphosphate (NTP) library for in vitro SELEX of RNA aptamers that bind to ATP (Jhaveri et al., Nat. Biotechnol. (2000) 18:1293-1297). To avoid multiple fluorescent residues in one aptamer, which may lead to non-detectable change in fluorescence signal upon target binding, only a small percentage of the NTP library is fluorescein-modified uridine. The selected aptamers were able to detect ATP in the μM range, with the best one containing only one fluorescein modified uridine. In addition, other fluorescent dyes can also be used to achieve similar signaling specificity and sensitivity.

Although sensors based on the “signal-off” mode can be less sensitive than those based on the “signal-on” mode, they can lead to better detection of targets with low-affinity aptamers. More importantly, simpler designs with fewer steps are needed for “signal-off” sensors, which can be convenient and cost-effective (Stojanovic et al., Am. Chem. Soc. (2001) 123:4928-4931; Liu et al., Anal. Chem. (2009) 81:2383-2387).

In general, a fluorescence donor and a quencher are conjugated respectively to both ends of the aptamer. Upon target binding, a conformational change of the aptamer repositions the donor and quencher in close proximity which leads to fluorescence quenching.

In some cases, the analyte (target) can quench the fluorescence of certain dyes after binding to an aptamer.

Another strategy for “signal-on” aptamer-based biosensors involves the use of label-free aptamer which, upon target binding, can displace fluorophores that are either previously quenched or of little fluorescence.

Gold nanoparticles and certain fluorophores can be quenched by nucleic acid bases. An ssODN containing an abasic site, such as a “dSpacer”, can be constructed and hybridized to an aptamer that binds a target of interest (Xiang et al., J. Am. Chem. Soc. (2009) 131:15352-15357). The resulting duplex can be embedded with 2-amino-5, 6, 7-trimethyl-1, 8-naphthyridine (ATMND) at the dSpacer which has its fluorescence quenched. Upon target binding, the aptamer undergoes structural changes and releases the dSpacer-containing ssODN, leading to fluorescence recovery of ATMND.

PCR-based or DHA-based approaches can also be employed to detect aptamer bound to its target. For example, two aptamers (one containing a structure such as a hairpin) that bind to a target of interest can be prepared. Upon target binding, structural changes occur in the aptamer, making a portion (e.g., 3′ end) of the aptamer available for priming binding and amplification by PCR. For example, after the aptamers are topically applied to the skin of a subject, a sample of the treated skin can be taken for primer binding and PCR amplification. See, He et al., Anal. Chem. (2010)82:1358-1364. A free portion (e.g., 3′ end) of the aptamer may be bound by a probe and detected by DHA.

For measurement of aptamer binding with small molecules, a displacement assay can be designed where a fluorescein labeled oligonucleotide is pre-hybridized with an aptamer, which is subsequently displaced by small molecules that can be recognized by the aptamer (Cruz-Aguado and Penner, Anal. Chem. (2008) 80:8853-8855). Similarly, an aptamer could be used to detect or evaluate a small molecule binding to its target. For example, a labeled aptamer whose fluorescence is quenched undergoes a structural change upon binding to its target, upon separation of the fluorophore and the quencher (through aptamer binding), the fluorescence signal is recovered which can be used for detection and qualitative or quantitative measurement of the target that is not bound to the small molecule. If the small molecule is bound to the target, the fluorophore on the aptamer will remain quenched.

All publications, patent applications, patents, and other references mentioned herein are incorporated by reference in their entirety. In the case of conflict, the present specification, including definitions, controls. In addition, the materials, methods, and examples are illustrative only and not intended to be limiting.

### EXAMPLES

The majority of the literature describing topical delivery of large molecular weight compounds relies on the use of animal models, where the skin is more permeable, and analytical techniques are limited in their ability to accurately assess the topical delivery and target engagement of these molecules in the skin. A 62-nucleotide (MW=20,395 Da) aptamer inhibitor (ARC32225) of IL23 that has high affinity and specificity to human IL23 cytokine has been developed. As shown herein, RNA based aptamers can penetrate human skin without physically disrupting the stratum corneum using a well validated model to assess passive topical delivery. Using novel analytical techniques to extract and detect the aptamer, the passive delivery can be quantified at therapeutic levels using different vehicles for delivery. These therapeutic levels were confirmed for biological activity with an ex vivo human skin model that can only be explained from a topical application. This is the first study showing the passive delivery of a large molecule across human skin capable of engaging a target to elicit a local biological response and represents a broad class of biological compounds that could open the field for local targeting of skin disease and diagnostics.

### Example 1: Selection and Initial Characterization of ARC32225

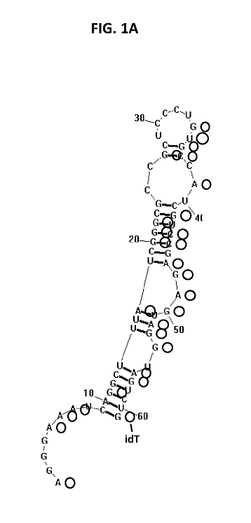

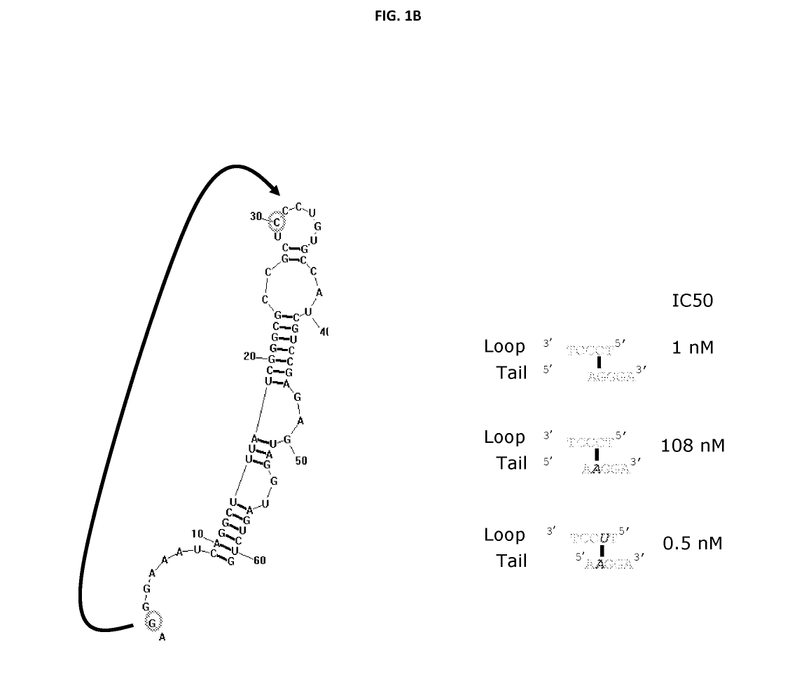

ARC32225 is a 62-residue oligonucleotide (FIG. 1A). This oligonucleotide is composed of ten 2′-O-methyladenosines (mA), one 2′-fluoroadenosine (fA), fourteen 2′-O-methylguanosines (mG), six 2′-fluoroguanosines (fG), three 2′-O-methylcytosines (mC), thirteen 2′-fluorocytosines (fC), one 2′-O-methyluridine (mU), thirteen 2′-fluorouridines (fU) and the 3′-terminus is capped with a single inverted deoxythymidine residue (idT) to protect against nuclease degradation. In vitro selection experiments to generate ARC32225 were conducted using a pool of modified oligonucleotide molecules, each of which was generated with mA, mG, fC and fU residues. Recombinant Interleukin 23 (IL-23) used in the selection was obtained from R&D Systems (catalog #1290-IL-010/CF).

Iterative rounds of selection for binding to IL-23 followed by amplification were conducted to generate ARC20122, an 84 nucleotide long precursor to ARC32225.

The sequence of ARC20122 is SEQ ID NO:5:

5′ mG-mG-mA-mG-mG-mG-mA-mA-mA-mA-mG-fU-fU-
mA-fU-fC-mA-mG-mG-fC-fU-fU-fC-fU-mA-fU-fC-
mG-mG-mG-fC-mG-fC-fC-mG-fC-mG-fU-fU-fU-fU-
fC-fC-fC-fU-mG-fU-mG-fU-fU-mA-mG-fC-fC-mA-
fU-fC-mG-fU-fC-fC-mG-mA-fC-mG-mA-mG-fU-mA-
mG-mG-fC-fU-mA-mG-fU-fC-fU-mG-mA-fU-mG-fC-
fC 3′

ARC20122 was minimized from 84 nucleotides to 61 nucleotides using computational folding prediction programs and systematic deletion. Modifications at the 2′ position of the sugar for each base was evaluated to improve activity as well as predicted in vitro stability either by substitution of a 2′Fluoro with a 2′-O-methoxyethyl(2′OME) (2′F->2′OMe) or substitution of a 2′-O-methoxyethyl with a 2′Fluoro (2′OMe->2′F). All tolerated or potency increasing substitutions were combined with all tolerated deletions within the oligonucleotide. Appendage of a 3′-inverted thymidine residue, to increase nuclease resistance, resulted in a 62 nucleotide long molecule (ARC32225). ARC32225 is also referred to in the Examples as “IL23 aptamer”, “IL23 Aptamer”, “IL-23 aptamer” or “IL-23 Aptamer”.

A tertiary structural interaction has been identified within ARC32225 between the G residue at position 2 and the C residue at position 30 (FIG. 1B). This interaction is substantiated by a site directed mutagenesis experiment. Substitution of an A residue at position 2 disrupts the predicted Watson-Crick base pairing and significantly reduces the activity of the aptamer to 108 nM in the STAT3 inhibition assay. A compensatory substitution of a U at position 30 that restores the Watson-Crick base pairing potential as well as inhibitory activity resulting in an IC₅₀ of 0.5 nM in the STAT3 assay.

Molecular Structure and Chemical Name: ARC32225 is the 62-mer oligonucleotide having the following sequence (SEQ ID NO:1):

5′-mA-fG-fG-fG-mA-mA-mA-fU-fC-fA-mG-fG-fC-
fU-fU-fU-mA-fU-fC-mG-mG-mG-fC-fG-fC-fC-mG-
fC-fU-fC-fC-fC-fU-mG-mU-mG-mC-fC-mA-fU-fC-
mG-fU-mC-mC-fG-mA-mG-mA-mG-fU-mA-mG-mG-fU-
mA-mG-fU-fC-fU-mG-idT-3′

The calculated molecular weight of ARC32225 in its free acid form is 20395.27 Da. The sodium salt of ARC32225 has a molecular weight of 21736.17 Da. The calculated extinction coefficient for the aptamer is 586,400 L mol⁻¹ cm⁻¹.

The K_{D} of the aptamer binding to recombinant human IL-23 was investigated in the presence of competitor tRNA. ARC20122, the full length parent to ARC32225, binds to recombinant human IL-23 with a K_{D} of 9.1 nM±2.5 nM. High affinity binding in the presence of 18,000 fold excess of nucleic acid competitor indicates the binding of ARC20122 is specific to IL-23 (data not shown).

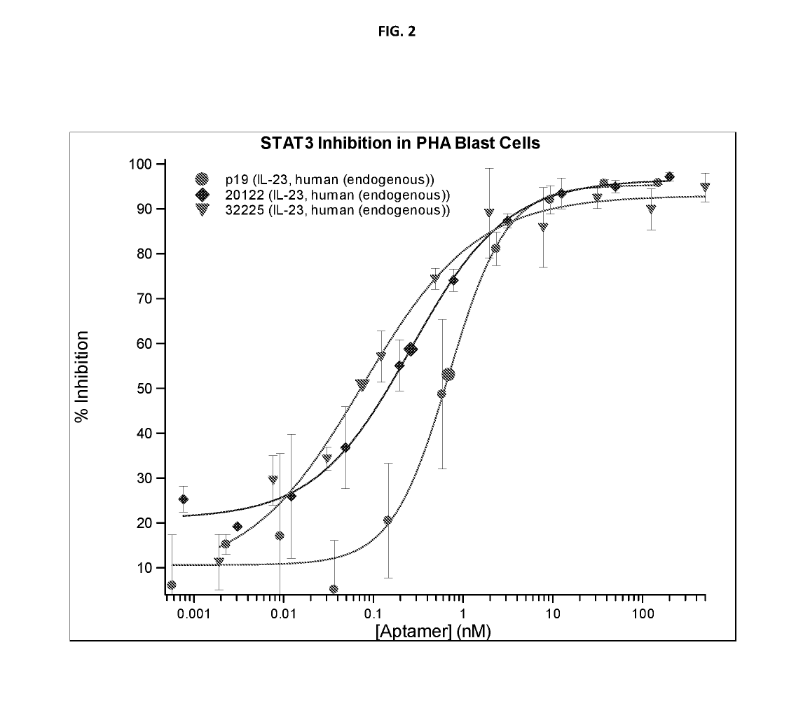

Additionally, the ability of ARC20122 as well as its minimer ARC32225 to inhibit IL-23 dependent stimulation of STAT3 activity in PHA-Blast cells was tested. This assay utilized endogenous IL-23 produced from purified monocytes as described herein. ACR20122 had an IC₅₀ of 0.26 nM±0.036 nM in this assay (FIG. 2). ARC32225 performed slightly better than its parent clone with an IC₅₀ of 0.075 nM±0.030 nM. In both cases, the activity was markedly better than the control anti-p19 antibody, IC₅₀=0.69 nM±0.12 nM.

ARC32225 was found to be resistant to serum nucleases. ARC32225 was incubated in 90% pooled human serum and rat serum for 24 hrs at 37° C. The relative amount of full length aptamer was visualized by SYBR gold staining of a 15% denaturing polyacrylamide gel. After a 24-hour incubation in rat serum, ˜50% of the full length aptamer remained intact. Significant amounts of full length aptamer remained after incubation in 90% human serum (data not shown).

### Example 2: Effects of ARC32225 on Endogenous IL-23 Activity

There are significant structural differences between available recombinant IL-23 and endogenous IL-23. Endogenous IL-23 was prepared from the conditioned media of activated purified primary human monocytes by affinity purification. Peripheral Blood Mononuclear Cells (PBMC) were obtained from buffy coats prepared from fresh human blood. Monocytes were isolated by adherence to CD14 coated magnetic beads (Miltenyi, Cologne, Germany). To induce and enhance IL-23 expression, monocytes were treated with LPS and anti-IL-10 blocking antibodies. After 24 hours, conditioned media was collected and frozen. Sepharose NHS agarose beads (Sigma, St. Louis, Mich.) were coupled with non-neutralizing p19 receptor monoclonal antibodies (eBioscience, San Diego, Calif.) and incubated overnight with pooled conditioned media from multiple donors. Beads were washed and endogenous IL-23 was eluted with acid, and neutralized with a TRIS-base, BSA containing buffer. The eluate was dialyzed with PBS, and the purified protein was aliquoted and frozen.

Expression of IL-23 from monocytes was confirmed by testing conditioned media using the p19/p40 ELISA from eBioscience (San Diego, Calif.). The ELISA plate was coated with an anti-p19 antibody and captured antigen was detected with an anti-p40 antibody, thus specifically measuring the IL-23 cytokine. With this ELISA, it was determined that IL-23 is present at quantities of approximately 8+/−4 ng/mL in the conditioned media and that the conditioned media had been depleted of IL-23 following incubation with the affinity beads.

Purified endogenous IL-23 activity was assessed by the protein's ability to activate STAT3 in PHA Blast lysates. A titration of purified protein was applied to IL-2/PHA activated T-cells for 15 minutes to induce the phosphorylation of STAT3. pSTAT3 (phospho-STAT3) levels in cell lysates were subsequently measured by an ELISA (Cell Signaling Technologies, Danvers, Mass.). The amount of phosphorylated STAT3 from PHA Blast cells treated with purified endogenous IL-23 was measured by colorimetric ELISA after 15 minutes of stimulation with dialyzed eluate. (data not shown). The endogenous protein was subsequently used in PHA Blast phospho-STAT3 assays at a volume falling in the linear range of the curve.

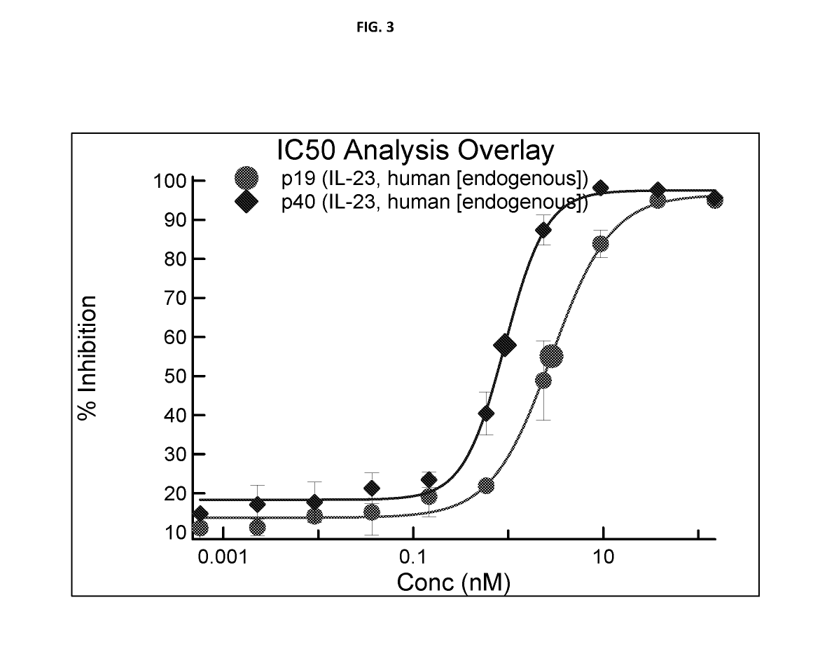

The majority of the phospho-STAT3 signal induction was attributed to purified endogenous IL-23. Both anti-p40 and anti-p19 neutralizing antibodies separately inhibit the signal by greater than 90% at 50 nM. Inhibition curves of the two antibodies are shown in FIG. 3.

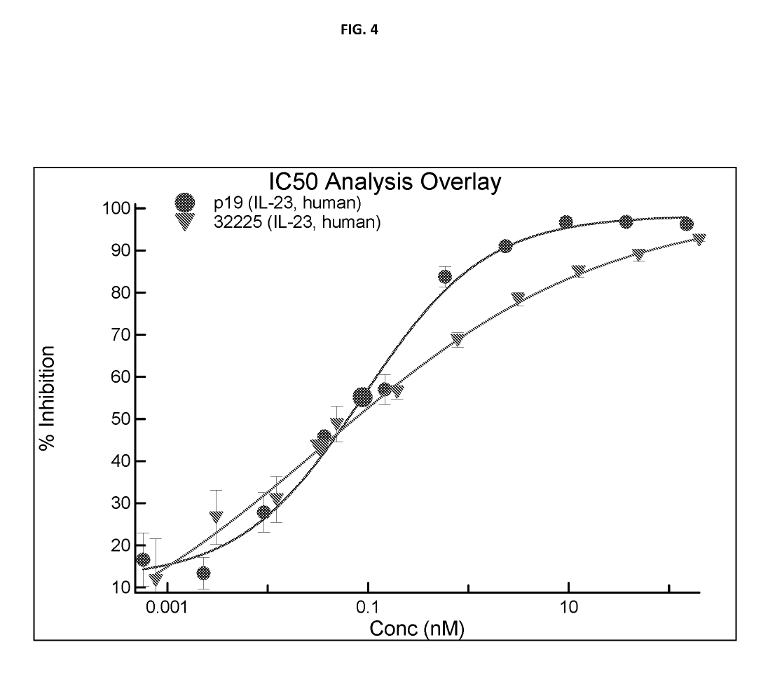

The ability of ARC32225 to inhibit endogenous IL-23 activation of PHA/IL-2 activated T-cells was tested by measuring levels of phospho-STAT3 (“phospho-STAT3 assay”). The binding of IL-23 to its receptor on activated T-cells stimulates several STAT pathways, including STAT3. Peripheral Blood Mononuclear Cells (PBMC) from fresh human blood were isolated by centrifugation in a Histopaque gradient and treated with PHA/IL-2 to enrich for T-cells and to upregulate IL-23 receptors. Cells were serum starved and treated with endogenous IL-23 for 15 minutes, and then lysed. Relative levels of phospho-STAT3 in IL-23 treated lysates, in the presence and absence of ARC32225 or a neutralizing p19 antibody, were measured by ELISA (Cell Signaling Technologies, Danvers, Mass.). Titrations of aptamer/antibody were used to generate inhibition curves from which IC₅₀ determinations were made. A representative graph of p19 and ARC32225 inhibition curves is shown in FIG. 4.

Seven 12-point titration curves, each in duplicate, were used to calculate an average (+/−SEM) IC50 of 2.0+/−1.2 nM for p19 and 0.296+/−0.090 nM for ARC32225. Percent inhibition was recorded for each concentration tested for all donors (data not shown). Individual IC50 for each donor were also recorded (data not shown).

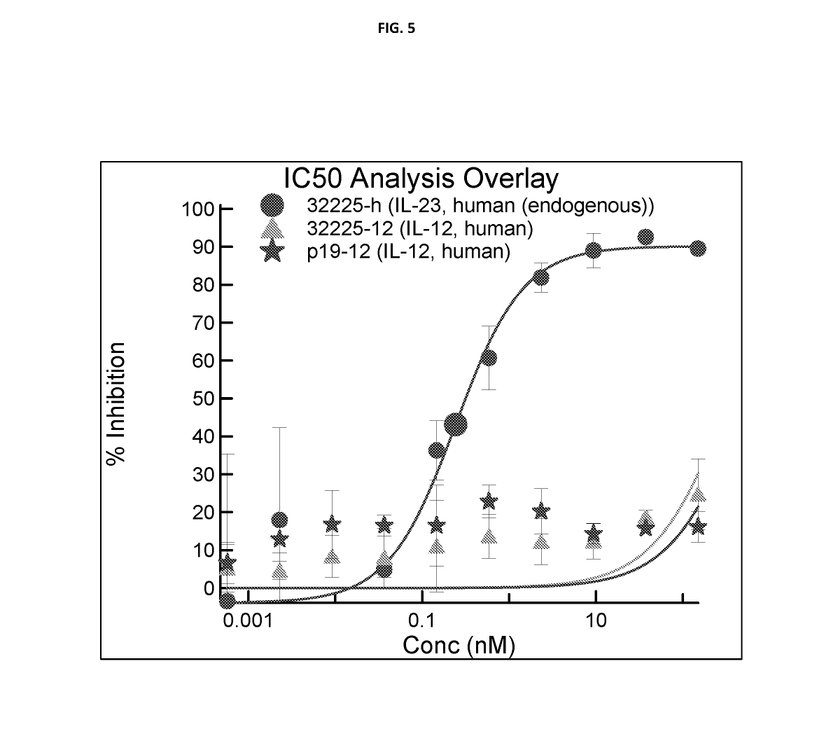

We also tested the ability of ARC32225 to inhibit IL-12 activation of PHA/IL-2 activated T-cells. Similar to IL-23, the binding of IL-12 to its receptor on activated T-cells stimulates several STAT pathways, including STAT3. The phospho-STAT3 assay was utilized to titrate IL-12 and a concentration in the linear range of STAT3 activation was chosen for use in future assays. Cells were serum starved and treated with IL-12 (20 ng/ml) for 15 minutes, and then lysed. Relative levels of phospho-STAT3 in IL-12 treated lysates, in the presence or absence of ARC32225 or a neutralizing p19 antibody, were measured by ELISA (Cell Signaling Technologies, Danvers, Mass.). Titrations of aptamer/antibody were used to generate an inhibition curves from which IC50 determinations were made. A representative graph of p19 and ARC32225 inhibition curves is found in FIG. 5.

### Example 3: Lack of TLR 3, 7, 8, or 9 Activation by ARC32225 In Vitro

The ability of ARC32225 to stimulate Toll-Like Receptors (TLR) 3, 7, 8 or 9 in human PBMCs was tested. The innate immune system mounts a rapid defense against invading bacteria, RNA and DNA viruses, and other pathogens. Some oligonucleotides are recognized by the innate immune system as potential pathogens stimulating an inflammatory response. TLR3 recognizes double stranded RNA and induces IL-6 release in plasmacytoid dendritic cells. Activation of TLR7/8 and 9 is sequence-dependent. Guanosine/uridine-rich single stranded RNA activates TLR7/8, whereas DNA containing unmethylated cytosine/guanosine hexamer motifs (CpGs) activates TLR9. Moreover, different classes of synthetic CpGs induce varying effects in PBMCs: Class A CpGs induce interferon alpha and interferon gamma expression and do not cause B cell proliferation. Class B CpGs induce a strong interleukin-6 response and initiate a proliferative response in B cells. The presence of CpG is not an absolute requirement for immune stimulation because sequences lacking CpGs have been reported to induce immune stimulation. Therefore, ARC3225 was screened for its ability to induce activation of the innate immune system.

ARC32225 was tested for its ability to induce IL-6 and interferon (IFN) alpha secretion, and B-cell proliferation in PBMCs. IL-6 release is a measure of TLR 3/7/8 and TLR9 Class B activation. B-cell proliferation is a measure of TLR9 Class B CpG stimulation. Interferon alpha release indicates TLR9 Class A CpG agonism. PBMCs were isolated by centrifugation in a Histopaque gradient and treated with 100 nM or 25 nM of ARC32225 or CpG controls. After 24 hours and 48 hours, supernatants were collected and assayed for IL-6 and IFN alpha release by ELISA. The amount of IFN alpha released correlates with absorbance at 450-600 nM by colorimetric ELISA. Cell proliferation was assessed from the replication of genomic DNA as measured by incorporation of the thymidine analog 5-bromo-2′deoxyuridine (BrdU). PBMCs were treated with 100 nM or 25 nM of aptamer or CpG B control for one day. Following a subsequent 24 hour pulse, cells were fixed and probed for BrdU using a chemiluminescent ELISA (Roche, Mannheim, Germany).

ARC32225 was not associated with immune stimulation of human PBMCs in vitro, as demonstrated by a lack of IL-6 or IFNα production or PBMC proliferation (data not shown). The PBMCs responded to the positive control oligonucleotides (CpG) with IL-6 and IFNα secretion and PBMC proliferation (data not shown).

### Example 4: Topical Application of IL23 Aptamer Penetrates Compromised and Uncompromised (Intact) Human Skin

The ability of the IL23 aptamer to penetrate human skin from topical application was first assessed fluorescently using freshly excised skin where the barrier was fully removed, partially compromised, or remained intact. The pretreated skin sections were mounted on flow-through diffusion cells using donor blocks to provide a leak proof seal, exposing a surface area of 1.0 cm². Diffusion cells were connected to multi-channel pumps with a flow rate of approximately 3.6 mL/hr with PBS. Each cell was then equilibrated in a heating manifold to ensure a skin surface temperature of 32° C. (for at least 30 min prior to dosing). ARC32225 was labeled with DyLight 488 (ARC32778), formulated into a cream at 11 mg/ml (˜1% v/v) and was spread uniformly onto the stratum corneum of the treated and untreated skin using a positive displacement pipette. The cream formulation was similar to the cream described in Mehta et al., J. Invest. Derma. 115:805-812 (2000). At zero and four hours post-application, the surface of the skin was washed using five cotton swabs soaked in a 0.05% v/v Tween 20/PBS solution to remove residual formulation from the surface of the skin. The washed skin was then frozen in OCT (Optimum Cutting Temperature) medium. Sections were mounted and fluorescence was detected by microscopy

The washing procedure was shown to be effective at removing aptamer from the surface of the skin in the zero hour samples where an absence of ARC32225 (as ARC32778) was observed in the intact and partially compromised skin (data not shown). Interestingly, the skin where the stratum corneum was completely removed, dosed topically, and immediately washed showed penetration of the ARC32225 (as ARC32778) aptamer into the epidermis with a gradient from the surface of the skin decreasing toward the basal membrane (data not shown). This suggests this aptamer can partition through the viable epidermis almost immediately and is not further limited by tight junctions in the epidermis, which are also thought to regulate passive diffusion. After four hours of topical exposure, ARC32225 (as ARC32778) aptamer was shown to penetrate all the skin samples (intact, tape stripped, and partially compromised) with a gradient observed from the surface of the skin toward the basal membrane with all samples (data not shown).

To confirm that the aptamer applied to and within the tissue blocks was intact labeled ARC32225 (ARC32778), the aptamer was extracted from two treated tissue blocks and then run on a PAGE against standard aptamers. Specifically, two tissue blocks corresponding to intact skin at 0 hrs and abraded skin at 4 hrs were left at room temperature for 10 minutes to melt the OCT. Each block contained two samples measuring ˜5-8 mm×2-3 mm each. One sample was refrozen and the other was minced with a razor blade and put into 200 uL of dPBS.

It was reasoned that the aptamers should be extracellular and that tissue disruption alone should release some of the aptamer delivered. The tissue was subjected to three freeze/thaw cycles in dPBS alone and vigorously vortexed. Ten microliters of each were loaded on a denaturing polyacrylamide gel. The isolated material was visualized fluorescently with a Molecular Dynamics PhosphorImager and migrated consistent with the known ARC 32778 standard (data not shown). These results confirm that the isolated aptamer is both full length and fluorescently labeled confirming the presence of intact aptamer in the skin.

### Example 5: Topical Application of IL23 Aptamer Penetrates Porcine Skin In Vivo

To determine if ARC32225 derivatives can penetrate living, porcine skin in a model for human psoriatic lesions, ARC32778 (Dylite 488 labeled ARC32225) was formulated into the same cream as used in the in vitro experiment at 11 mg/ml. A 100 ul of test article was applied to a 1″×1″ square area on the back of tape-stripped (20 applications of scotch tape) or untape-stripped female white Yucatan miniature swine. After 0 or 4 hours, full thickness biopsies were collected from the designated dose sites. Each 0-hour biopsy was collected by using a #10 scalpel blade. Each tissue was then placed into a 2 ml cryovial and flash frozen in liquid nitrogen. Four-hour biopsies were taken in the same way after each animal was euthanized with an over dose of sodium pentobarbital.

The biopsies were analyzed by fluorescent microscopy. Usable images were only obtained for two samples (data not shown). In the tape-stripped 4-hour sample, significant amounts of ARC32778 are seen throughout the epidermis to the dermal layer excluded from the cellular nuclei (data not shown). In contrast, no fluorescence was seen beyond the stratum corneum in the untape-stripped, 0-hour control (data not shown). These preliminary results are consistent with the in vitro study and suggest that the aptamer is capable of penetrating and residing for 4 hours into live, tape-stripped porcine skin.

### Example 6: Aptamer Preparation

The ARC32225 aptamer is a synthetically manufactured phosphodiester oligonucleotide incorporating nucleotides with a combination of 2′-fluoro and 2′-O-methyl modifications. ARC32225 consists of the core 61-mer oligonucleotide. The 3′-terminus carries an additional inverted dT cap as protection against nuclease degradation. The aptamer can be presented as a lyophilized powder. The starting materials and reagents, solvents and auxiliary materials used in the manufacture are shown in Tables 2 and 3.

TABLE 2
Starting Materials Used in Drug Substance Manufacture

| | |
|---|---|
| 2′-OMe G(ibu)-CE, phosphoramidite: [5′-Dimethoxytrityl-N- | mG |
| isobutyryl-guanine, 2′-O-methyl-3′-[(2-cyanoethyl)-(N,N- | |
| diisopropyl)]-phosphoramidite] | |
| 2′-OMe A(bz)-CE, phosphoramidite: [5′-Dimethoxytrityl-N- | mA |
| benzoyl-adenine, 2′-O-methyl-3′-[(2-cyanoethyl)-(N,N- | |
| diisopropyl)]-phosphoramidite] | |
| 2′-OMe C(Ac)-CE, phosphoramidite: [5′-Dimethoxytrityl-N-acetyl- | mC |
| cytosine, 2′-O-methyl-3′-[(2-cyanoethyl)-(N,N-diisopropyl)]- | |
| phosphoramidite] | |
| 2′-OMe U-CE, phosphoramidite: [5′-Dimethoxytrityl-uracil, 2′-O- | mU |
| methyl-3′-[(2-cyanoethyl)-(N,N-diisopropyl)]-phosphoramidite] | |
| 2′-Fluoro-ibu-dG-CE, phosphoroamidite: [5′-Dimethoxytrityl-N- | fG |
| isobutyryl-deoxyGuanine, 2′-fluoro-3′-[(2-cyanoethyl)-(N,N- | |
| diisopropyl)]-phosphoramidite] | |
| 2′-Fluoro-Bz-dA-CE, phosphoroamidite: [5′-Dimethoxytrityl-N- | fA |
| benzoyl-deoxyAdenine, 2′-fluoro-3′-[(2-cyanoethyl)-(N,N- | |
| diisopropyl)]-phosphoramidite] | |
| 2′-Fluoro-Ac-dC-CE, phosphoroamidite: [5′-Dimethoxytrityl-N- | fC |
| acetyl-deoxyCytosine, 2′-fluoro-3′-[(2-cyanoethyl)-(N,N- | |
| diisopropyl)]-phosphoramidite] | |
| 2′-Fluoro-dU-CE, phosphoroamidite: [5′-Dimethoxytrityl-N- | fU |
| deoxyUracil, 2′-fluoro-3′-[(2-cyanoethyl)-(N,N-diisopropyl)]- | |
| phosphoramidite] | |
| 5′-Inverted dT Controlled Pore Glass support: [3′-dimethoxy- | idT- |
| trityl-2′-deoxyThymidine, 5′-succinoyl-long chain | CPG |
| alkylamino-CPG] | |

TABLE 3
Reagents, Solvents and Auxiliary Materials Used in the
Drug Substance Manufacture

Oxidizer solution [50 mM iodine in pyridine/water (9:1 v/v)]
5-(Ethylthio)-1H-tetrazole (S-ethyl-tetrazole, SET)
Dichloroacetic acid/dichloromethane, 0.36M DCA (3% v/v DCA/DCM,
Detritylation Solution)
Cap A solution (20% N-methylimidazole in ACN)
Cap B solution (20% isobutyric anhydride, 30% 2,6-lutidine in ACN)
Acetonitrile (ACN)
Ammonium hydroxide, 28-30% in water
Dimethylsulfoxide (DMSO)
Di-sodium hydrogen phosphate (Na₂HPO₄)
Ethanol (EtOH)
N-Methylpyrrolidinone (NMP)
Sodium acetate (NaOAc)
Sodium di-hydrogen phosphate, monohydrate (NaH₂PO₄ × H₂O)
Sodium chloride (NaCl)
Sodium hydrogen carbonate (NaHCO₃)
Water for Injection (WFI) quality water
Acetic acid

Synthesis:

ARC32225 was synthesized at 2.5 mmol scale on an ÄKTA Oligopilot 100 automated solid-phase synthesizer using the phosphoramidite method analogous to standard protocols (Beaucage, S. L.; In Current Protocols in Nucleic Acid Chemistry; Beaucage, S. L., Bergstrom, D. E., Glick, G. D., Jones, R. A., Eds.; John Wiley & Sons: New York, N.Y., 2001; Chapter 3.).

A fixed volume synthesis column (4.5×10 cm) is loaded with solid support derivatized with the first nucleoside residue (idT), and is connected to the synthesizer. Synthesis is initiated when detritylation solution is pumped through the column to remove the acid labile dimethoxytrityl group to generate the 5′-OH of the first nucleoside. For the synthesis of ARC32225, it is beneficial to add an additional acid treatment for the inverted deoxythymidine on the solid support, and for the pyrimidine bases. In addition, the UV cutoff is lowered from 500 mAU to 100 mAU during the watch cycle. Overall, the number of n-x failures is reduced with the additional acid treatment. Phosphoramidite in acetonitrile and activator (SET) are then delivered simultaneously to the synthesis column resulting in coupling of the amidite to the 5′-hydroxyl. For ARC32225, 3.0 equivalents of 2′-fluoro and 2′-O-methyl amidites, were found to produce consistent yield and FLP. Following coupling, the column is then washed with acetonitrile. Prior to oxidation, 1 CV (column volume) of Cap B solution treats the column to ensure the support remains basic during the oxidation. Following the Cap B treatment, oxidizing solution is pumped through the column to convert the phosphite triester linkage P(III) into its phosphotriester analog P(V). After an acetonitrile wash, residual unreacted 5′-hydroxyl groups are capped using a mixture of Cap A and Cap B solutions. This step prevents any unreacted 5′-OH from further reactions in the synthesis cycle and thus unwanted chain growth of deletion sequences. isobutyric anhydride was preferential to the standard acetic anhydride solution for the synthesis of ARC32225 in terms of both yield and FLP (full length product).

After acetonitrile washes, a new elongation cycle is initiated with another detritylation step to generate the 5′-OH of the growing nucleotide chain, followed by the next phosphoramidite incorporation. This process is repeated until the desired sequence has been synthesized. The synthesis concludes with the addition of the terminal hexylamine modifier phosphoramidite.

Base Wash, and Cleavage/Deprotection:

On completion of the synthesis, the solid support is washed with a solution of 20% diethylamine, in acetronitrile, to remove the cyanoethyl protecting groups on the phosphate backbone. The solid support is then connected to a vacuum and excess solvent is removed. The solid support is then transferred to a deprotection vessel; saturated ammonium hydroxide is added and the mixture is heated to approximately 45° C. for 14-16 hours to affect cleavage from the solid support, and deprotection of the exocyclic base protecting groups. The deprotection is allowed to cool to ambient temperature and the solid support is filtered utilizing a sintered glass funnel, rinsed with water and ethanol, and carried forward into the following step. Both the base wash and the cleavage/deprotection were optimized to maximize yield and minimize impurities. In addition, limits on both time and volume were set.

Trityl-on Purification of ARC32225:

ARC32225 was purified using RP chromatography. The final 5′ trityl was left on during synthesis to help resolve FLP from n-x impurities. For the reverse phase chromatography process, a silica gel matrix was used with the C4 alkyl group ligands immobilized to silanol hydroxyl groups on the gel (Sepax Technologies, Inc.). The column is loaded at <18 mg/mL at >40° C. where separation is accomplished with a gradient of increasing hydrophobicity.

Trityl Removal and Counterion Exchange of ARC32225:

Purified material from the RP chromatography is pooled and loaded at ˜20 mg/mL at >40° C. onto an agarose matrix IEX column (Q-Sepharous, GE Amersham) for trityl removal and backbone counter ion exchange. Loaded material is exposed to 80% acetic acid at 25° C. and UV monitoring is used to determine completion of trityl-off step. The column is neutralized to pH 6-7 and then FLP is eluted in a buffered aqueous solution of increasing ionic strength at >40° C.

Ultrafiltration:

Following the column purification of ARC32225, the solution is loaded onto an ultra filtration apparatus (AKTA Crossflow, GE Lifesciences) outfitted with a 5 KDa MWCO (molecular weight cut off) membrane and the solution is diafiltered with water to a constant conductivity to remove excess salts.

Lyophilization:

The purified and desalted solution of ARC32225 is transferred into LyoGuard trays (Gore Associates). The product is then freeze-dried to a dry off-white to slightly yellow powder.

### Example 7: Dual Hybridization Assay (DHA) Development

The pharmacokinetics of an unPEGylated aptamer dosed via intravenous (IV) or subcutaneous (SC) injection have been measured historically with HPLC. HPLC is sufficient for quantitation of aptamer in these circumstances due to the relatively high concentration of aptamer injected during the studies. However, aptamers can also be administered topically. Given the presumed low dosing concentration and penetration of topically administered ARC32225 into the skin, the concentration of aptamer in a given tissue or plasma sample is likely to be below the lower limit of quantitation (LLOQ) of the HPLC (approximately 10×10⁻⁹ to 50×10⁻⁹M). For this reason, the Dual Hybridization Assay (DHA) was developed.

In the DHA, complementary base pairing drives specificity and sensitivity resulting in quantitation of ARC32225 concentrations in the range of 10×10⁻¹² to 40×10⁻¹² M. Briefly, detection and capture probes were synthesized that are complementary to the 5′ and 3′ ends of the aptamer, respectively. The probes are designed such that the capture probe has a free amine at its 5′terminus. This free amine allows the probe to be covalently linked to a 96-well plate (Nunc Immobilizer, #436006). The detection probes are designed with a biotin molecule covalently attached to the 3′terminus of the probe to allow detection by streptavidin conjugated horseradish peroxidase using a tetramethylbenzidine (TMB) substrate. Excess detection probe is incubated with an aptamer standard or PK sample and the solution is heated allowing unfolding of ARC32225. Following this melting step, the solution is slowly cooled, thus permitting complementary base pairing between the detection probe and aptamer. The solution is then added to the capture probe already covalently attached to the 96-well plate. Through this approach, the concentration of an aptamer in a sample can be determined by comparison to a standard curve of the aptamer included in the same assay.

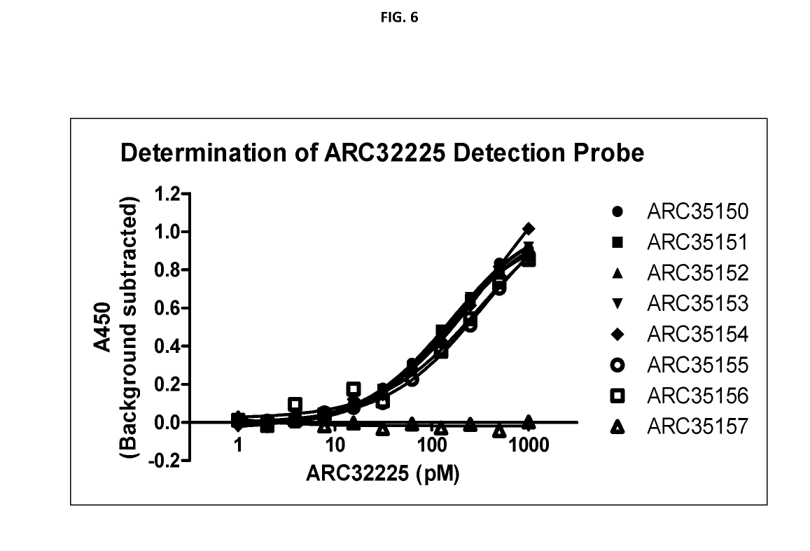

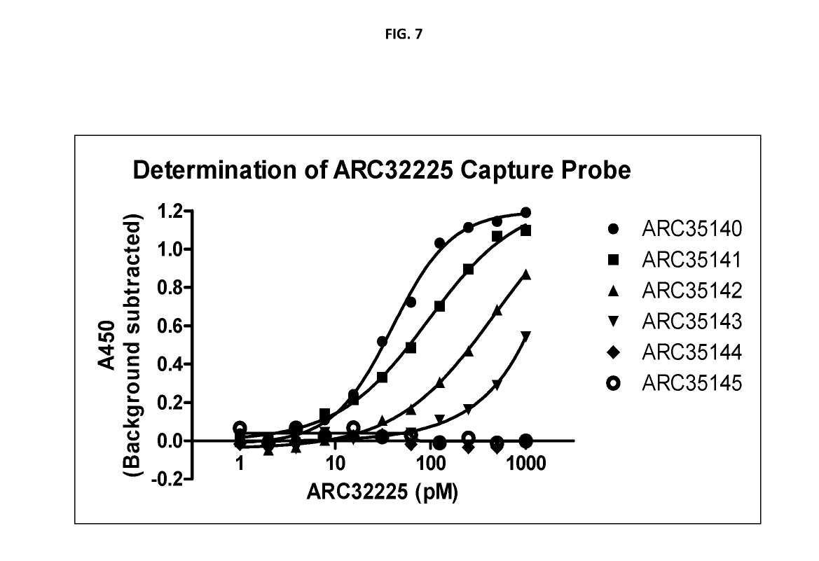

Development of the assay began by determining the optimal ARC32225 capture and detection probes, ARC35141 and ARC35156, respectively (FIGS. 6 and 7). ARC35141 is a 15 nucleotide probe that includes a 4 nucleotide spacer between the free amine at the 5′-end of the 11 nucleotides providing complementary base pairing. ARC35156 is a 13 nucleotide probe that includes a 4 nucleotide spacer between the biotin at the 3′terminus of the probe and the nine nucleotides providing complementary base pairing.

The detection probes evaluated are shown below. The results for each probe are shown in FIG. 6. Evaluation of the biotinylated detection probes demonstrated that all probes behaved similarly with the exception of ARC35157 which failed to hybridize with ARC32225. To balance sensitivity and specificity, ARC35156, which functioned similar to longer probes, was chosen as the detection probe (n=1).

The capture probes evaluated are shown below. The results for each probe are shown in FIG. 7. Using ARC35156 for detection of ARC32225, several ARC32225-specific capture probes were assessed. As the length of the capture probe decreases (ARC35140 contains the most complementary bases whereas ARC35145 contains the fewest) there is a decrease in capture of ARC32225. Although there is a slight difference in the sensitivity of ARC35140 and ARC35141, this difference is minimal and the sensitivity of the two probes is similar. Given this and the advantage that a shorter capture probe will provide in specificity, ARC35141 was chosen as the capture probe (n=1). The DHA was shown to be selective against the IL23 where single removals of nucleotides from the aptamer or the probes resulted in a loss of detection. Complete loss was observed when two or more nucleotides are removed at the concentration range of <1,000 pM.

Detection probes:

| | | |
|---|---|---|
| ARC35150 | 5′ mA mA mG mC mC mU mG mA mU mU mU mC mC mC mU mU mU mU mU biotin 3′ | SEQ ID NO: 6 |
| ARC35151 | 5′ mA mG mC mC mU mG mA mU mU mU mC mC mC mU mU mU mU mU biotin 3′ | SEQ ID NO: 7 |
| ARC35152 | 5′ mG mC mC mU mG mA mU mU mU mC mC mC mU mU mU mU mU biotin 3′ | SEQ ID NO: 8 |
| ARC35153 | 5′ mC mC mU mG mA mU mU mU mC mC mC mU mU mU mU mU biotin 3′ | SEQ ID NO: 9 |
| ARC35154 | 5′ mC mU mG mA mU mU mU mC mC mC mU mU mU mU mU biotin 3′ | SEQ ID NO: 10 |
| ARC35155 | 5′ mU mG mA mU mU mU mC mC mC mU mU mU mU mU biotin 3′ | SEQ ID NO: 11 |
| ARC35156 | 5′ mG mA mU mU mU mC mC mC mU mU mU mU mU biotin 3′ | SEQ ID NO: 12 |
| ARC35157 | 5′ mA mU mU mU mC mC mC mU mU mU mU mU biotin 3′ | SEQ ID NO: 13 |

Capture probes:

| | | |
|---|---|---|
| ARC35140 | 5′ NH2 mU mU mU mU mC mC mG mA mC mU mA mC mC mU mA mC 3′ | SEQ ID NO: 14 |
| ARC35141 | 5′ NH2 mU mU mU mU mC mC mG mA mC mU mA mC mC mU mA 3′ | SEQ ID NO: 15 |
| ARC35142 | 5′ NH2 mU mU mU mU mC mC mG mA mC mU mA mC mC mU 3′ | SEQ ID NO: 16 |
| ARC35143 | 5′ NH2 mU mU mU mU mC mC mG mA mC mU mA mC mC 3′ | SEQ ID NO: 17 |
| ARC35144 | 5′ NH2 mU mU mU mU mC mC mG mA mC mU mA mC 3′ | SEQ ID NO: 18 |
| ARC35145 | 5′ NH2 mU mU mU mU mC mC mG mA mC mU mA 3′ | SEQ ID NO: 19 |

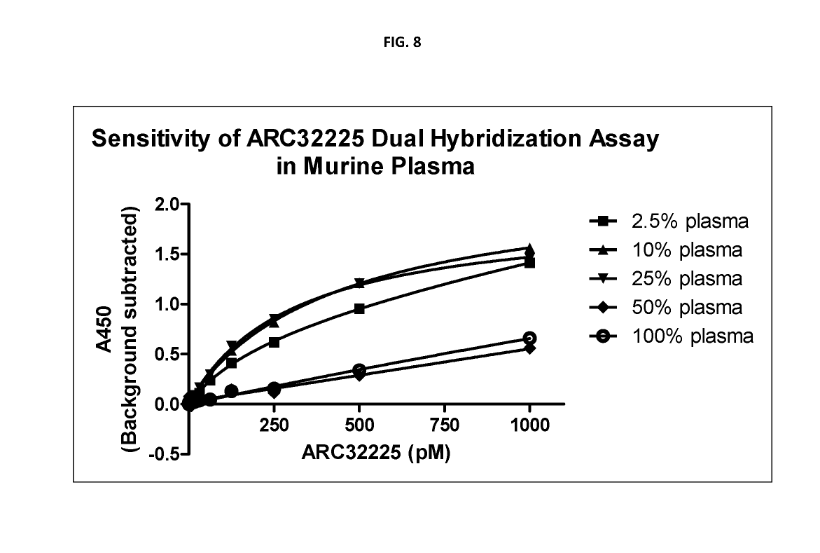

The sensitivity of the assay in murine, porcine and cynomolgus plasma was determined. To determine the LLOQ of the assay in each plasma, assay sensitivity was determined in different concentrations of species-specific plasma. When plasma is present, it is necessary to be cognizant of the percentage of plasma within each sample. At plasma concentrations >25%, the samples cannot be heated above 65° C. to melt the aptamer or else the protein from the plasma will congeal. Samples that contain ≦25% plasma can be heated to 95° C. in order to melt the aptamer. As a result of these studies, the LLOQ of ARC32225 was determined for each species-specific plasma using ARC35141 and ARC35156 as probes. Approximately 5×10⁻¹² to 10×10⁻¹² M ARC32225 could be detected in 25% murine plasma (FIG. 8). In 50% or 100% murine plasma, the dual hybridization signal was weak and determined to be insufficient for confident quantitation of ARC32225. Thus, the LLOQ of ARC32225 in murine plasma is approximately 20×10⁻¹² to 40×10⁻¹² M. As shown in the results of FIG. 8, in order to predict the LLOQ of ARC32225 detectable in murine plasma, ARC32225 was titrated in various concentrations of diluted plasma using the capture and detection probes previously described. In 25% murine plasma, the sensitivity of the assay is ˜5-10 pM, thus the LLOQ is ˜20-40 pM (n=1).

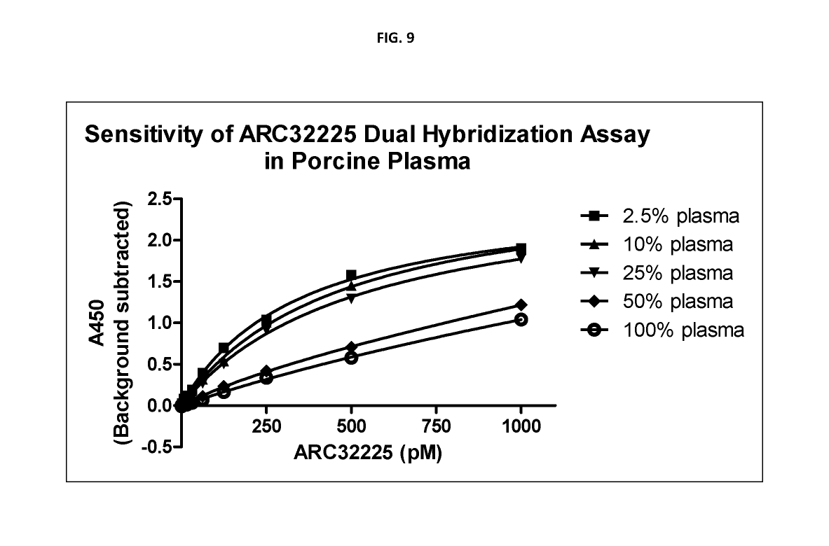

In porcine plasma, similar results were obtained with 25% plasma representing the highest concentration of plasma that should be used, whereas 50% and 100% porcine plasma resulted in a weak signal (FIG. 9). Additionally, the LLOQ of ARC32225 in porcine plasma was approximately 20×10⁻¹² to 40×10⁻¹² M, similar to that in murine plasma. As shown in the results of FIG. 9, in order to predict the LLOQ of ARC32225 detectable in porcine plasma, ARC32225 was titrated in various concentrations of diluted plasma using the capture and detection probes previously described. In 25% porcine plasma, the sensitivity of the assay is ˜5-10 pM, thus the LLOQ is ˜20-40 pM (n=1).

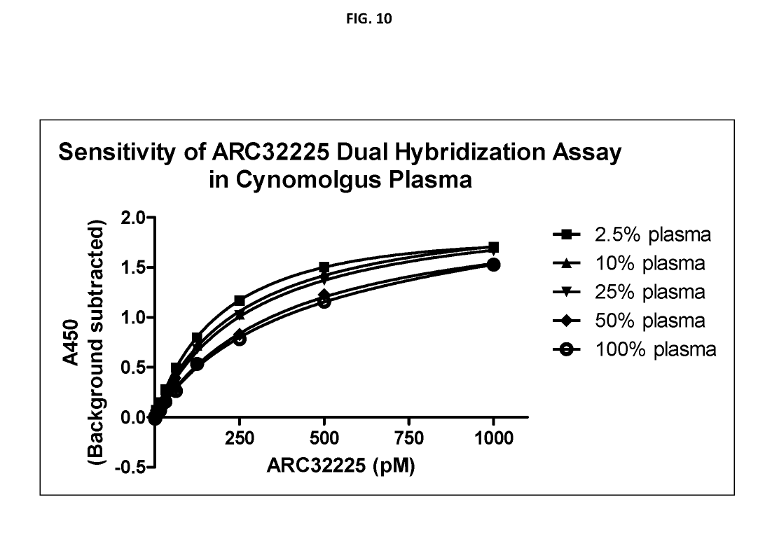

In cynomolgus monkey plasma, ARC32225 demonstrated a stronger signal in 50% and 100% plasma, suggesting that a larger range of monkey plasma samples could be used (FIG. 10). Based on this data, the LLOQ of ARC32225 in monkey plasma is calculated to be approximately 10×10⁻¹² to 20×10⁻¹²M. As shown in the results of FIG. 10, in order to predict the LLOQ of ARC32225 detectable in cynomolgus plasma, ARC32225 was titrated in various concentrations of diluted plasma using the capture and detection probes previously described. In 50% cynomolgus plasma, the sensitivity of the assay is ˜5-10 pM, thus the LLOQ is ˜10-20 pM (n=1).

Thus, in all plasmas tested, the dual hybridization assay will enable quantitation of ARC32225 concentrations approximately 1000-fold lower than via HPLC methods.

### Example 8: Topical Delivery of IL23 Aptamer and Cargo Aptamers Through Human Skin and into Keratinocytes

The ability of IL23 (IL-23) aptamer (ARC32225) to permeate skin was visually assessed using fluorescence after topical application on human ex vivo skin. The size (molecular weight) and broadness of this permeation was also assessed using fluorescence with three different cargo aptamers and a range of molecular weights. Cargo aptamer 1 was generated at two sizes (39-mer and 85-mer) to investigate the effect size on the passive permeation of aptamers. A second cargo aptamer was generated at three sizes (36-mer, 45-mer, and 86-mer) to explore the broadness of aptamer passive permeation and the impact of size. Finally a third cargo aptamer (85-mer) was included as a third aptamer for comparison. All three aptamers were designed as cargo aptamers with no therapeutic activity, therefore the biological activity could not be explored.

Methods

Freshly excised human abdominal skin was dermatomed to a thickness of 500±100 μm and mounted on flow-through diffusion cells using donor blocks to provide a leak proof seal, exposing a surface area of 1.0 cm². Diffusion cells were connected to multi-channel pumps with a flow rate of approximately 3.6 mL/hr with PBS. Each cell was then equilibrated in a heating manifold to ensure a skin surface temperature of 32° C. (for at least 30 min prior to dosing). IL23 and the cargo aptamers were labeled with Cy3 and were dissolved in an aqueous vehicle (97.9% DI Water, 0.1% Polysorbate 20, 1% Benzoyl alcohol). The aqueous formulations were applied topically at a dose of 10 uL/cell (10 mg/cm2), which was spread uniformly onto the stratum corneum surface using a positive displacement pipette. At zero, six, and twenty four hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. The dried tape-stripped skin was placed epidermis side down on a clean cutting board and a clean razor blade was used to cut smaller sections. These sections were placed on edge in 15×15 mm moulds and then immediately frozen in OCT using an isopentane/dry ice bath. The moulds were then removed from the isopentane bath, wrapped in aluminium foil, and stored at −80° C. until Immunofluorescence (IFC) was analyzed. Frozen tissue blocks were removed from the −80° C. freezer and placed into −20° C. cryostat 3 minutes prior to sectioning. Five μm sections were cut and mounted on individual Richard Allen Bond-Rite slides. The slides were fixed in cold −20° C. acetone for 10 min and then air dried for 30 minutes. The slides were rinsed in Tris Buffered Saline (TB S) (Dako Ref S3006) with three changes of TBS. All slides were coverslipped with PROLONG® Gold Antifade Reagent with diamidino-2-phenylindole (DAPI) (Invitogen, P36935). The amount of permeation was assessed using a fluorescence microscope. Photographic images were taken using a Leica DMRXA2 microscope mounted with a Nikon DS-QiMc monochromatic camera and NIS Elements BR 3.2.

Results

IL23 Cy3 Labelled Aptamer:

The washing procedure was again shown to be effective at removing aptamer from the surface of the skin in the zero hour samples (data not shown). IL23 aptamer was shown to penetrate the epidermis without any barrier disruption as early as 6 hours with increased dramatically by twenty four hours (data not shown). Several areas throughout the skin sections showed fluorescence signal which appeared to be within the keratinocytes under the fluorescent microscope (data not shown). Confocal imaging showed the IL23 aptamer was within the cells and potentially within the nucleus of the keratinocytes (data not shown). The penetration of the IL23 aptamer in human skin has been repeated with a different fluorescently label and the deliver appears to be intracellular and extracellular.

Cy3 Labelled Cargo Aptamers:

Cargo aptamer 1 was generated at two sizes (39-mer and 85-mer) to investigate the effect size on the passive permeation of aptamers. A second cargo aptamer was generated at three sizes (36-mer, 45-mer, and 86-mer) to explore the broadness of aptamer passive permeation and the impact of size. Finally a third cargo aptamer (85-mer) was included as a third aptamer for comparison. All three aptamers were designed as cargo aptamers with no therapeutic activity, therefore the biological activity could not be explored.

All three cargo-based aptamers resulted in significant permeation throughout the epidermis with an increase in fluorescence from 6 to 24 hours. It was difficult to quantify, but there did not appear to be an effect of size as the 36-mer cargo aptamer showed little differentiation compared to the 86-mer cargo aptamer. Many of the sections show fluorescence that appeared to be intracellular, suggesting these aptamers are capable of engaging both intracellular or extracellular targets. Further, these results suggest the passive delivery of aptamers may be a more broad based phenomenon outside of the IL23 aptamer.

### Example 9: Creams and Microemulsions

Simple solvent systems showed that passive permeation of a large molecular weight aptamer is possible, where vehicles with higher water concentrations seemed to have an increased delivery to the dermis. Water-in-oil microemulsions have been suggested as an effective means of topical delivery of peptides and proteins and may have a much more generic role in the delivery of a wide range of water-soluble molecules [7]. It was of interest to see if microemulsions could be used to improve delivery of the IL23 aptamer (ARC32225). Mehta, et. al. showed that a cream vehicle could deliver a large molecular weight anti-sense oligonucleotide into human skin transplanted on severe compromised immunodeficient mice [8]. Given this IL23 is also an oligonucleotide; the cream vehicle was used for comparison to the microemulsions. The aim of this study was to compare a dose response in microemulsions to a aqueous based cream from the literature.

Methods

Freshly excised human abdominal skin was dermatomed to a thickness of 500±100 μm and mounted on flow-through diffusion cells using donor blocks to provide a leak proof seal, exposing a surface area of 1.0 cm². Diffusion cells were connected to multi-channel pumps with a flow rate of approximately 3.6 mL/hr with PBS. Each cell was then equilibrated in a heating manifold to ensure a skin surface temperature of 32° C. (for at least 30 min prior to dosing). IL23 aptamer (ARC32225) formulations were applied topically at a dose of 10 uL/cell (10 mg/cm2), which was spread uniformly onto the stratum corneum surface using a positive displacement pipette. The composition of the topical vehicles can be found in Table 4. Test articles were applied to one to three separate donors and at least 5 replicates per skin donor. At six and twenty four hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. Epidermis and dermis samples from skin penetration studies were placed into separate vials and 1 mL of 0.25% trypsin was added individual to the epidermis and dermis respectively. Vials were placed in oven set at 37° C. for 16 to 20 hours and then the extraction diluent was removed and centrifuged at 13,000 RPM for 10 min to remove all un-dissolved materials and particles.

TABLE 4
Composition of cream and microemulsions

| | Formulation Name | | | | |
|---|---|---|---|---|---|
| | 0.50% | 0.75% | 0.45% | 1% | |
| | Microemulsion | Microemulsion | Microemulsion | Microemulsion | 1% Cream |
| | SRA Formulation Number | | | | |
| | 26060-09-02 | 26060-09-04 | 26053-10-03 | 26954-05-02 | 26053-10-01 |
| Ingredient | % w/w | % w/w | % w/w | % w/w | % w/w |
| Aptamer IL23 | 0.53 | 0.75 | 0.45 | 1.00 | 1.00 |
| Caprylic/Capric Triglyceride | 60.00 | 56.38 | — | — | — |
| Glyceryl Monocaprylate | 20.00 | 18.79 | — | — | — |
| Polysorbate 80 | 8.84 | 8.31 | 34.35 | — | — |
| Sorbitan Oleate | 5.90 | 5.54 | — | — | — |
| Isopropanol | — | 3.46 | — | 5.0 | — |
| Demineralized Water | 4.73 | 6.77 | 11.09 | 20.0 | 62.50 |
| Benzyl Alcohol | — | — | — | 1.5 | — |
| Drakeol-5 | — | — | — | 30.0 | — |
| Brij O2 | — | — | — | 15.0 | — |
| Brij O10 | — | — | — | 15.0 | — |
| Cetrimonium chloride 30% solution | — | — | — | 12.5 | — |
| Propylene Glycol Monocaprylate | — | — | 10.91 | — | — |
| Propylene Glycol Dicaprylocaprate | — | — | 31.81 | — | — |
| Diethyleneglycol Monoethyl Ether | — | — | 11.39 | — | — |
| Benecel MP333C | — | — | — | — | 0.50 |
| Isopropyl Myristate | — | — | — | — | 10.00 |
| Glyceryl Monostearate NSE 400 | — | — | — | — | 10.00 |
| Polyoxyl-40-stearate | — | — | — | — | 15.00 |
| Phenonip | — | — | — | — | 1.00 |
| Total | 100.00 | 100.00 | 100.00 | 100.00 | 100.00 |

Dual Hybridization Assay:

The dual hybridization assay (DHA) has been previously described for quantifying plasma and vitreous humor samples [9]. See also Example 7 herein. Briefly, this assay is analogous to an enzyme-linked immunosorbent assay (ELISA) where aptamer is allowed to hybridize to a capture and detection deoxyribonucleic acid (DNA) oligonucleotide probes. The capture probes (ARC35141; Integrated DNA Technologies) to the 13 nucleotides at the 5′-terminus of the aptamer and was covalently attached to a plate via a 3′-terminal amino linker. The detection probe (ARC35156; Integrated DNA Technologies) was complementary to the 15 nucleotides at the 3′-terminus of the aptamer and was biotinylated at the 5′-terminus. This assay has been shown to have the selectivity and sensitivity to be able to extract aptamer from the skin tissue and quantify at the picomolar concentrations.

DHA assay plates were prepared by coating 96 well plates with 100 μL of capture probe (250 nM in 1 mM EDTA/DPBS) and incubated at 4° C. overnight. The capture probe solution was removed and the plate was washed using 3×300 μL of wash buffer (tris buffered saline with Tween (TBST)) with a minimum of 1 minute soaking time for each of the 3 washes cycles and the plate was tapped out on absorbent paper after each wash. Following removal of wash buffer, 200 μL of the blocking solution (5% w/v BSA in TBST) was added to each well and the assay plate was sealed and incubated at room temperature for 60 minutes on an orbital shaker. The blocking solution was removed and the plate was washed as previously described. An aptamer standard curve of aptamer was prepared by doubling dilutions to produce standards from 10 nM to 10 pM and DPBS as a blank sample (0 pM) was added in duplicate (60 μL per well) to a fresh 96 well plate (hybridisation plate). The extracted epidermis and dermis samples were added to the hybridisation plate (60 μL). To each well of hybridisation plate containing samples and standards, 60 μL of the detection probes (150 nM in DPBS) was added. The final concentration of detection probes was 75 nM and aptamer standard concentrations were from 0 pM, 5 nM to 5 pM. The hybridization plate was sealed and placed into a heated oven set to 65° C. for 40 minutes to allow for the aptamer to hybridize with the probes, and then allowed to cool to room temperature. 100 μL of the aptamer standard/sample:detection probe mixture was transferred from the hybridization plate to a DHA assay plate, sealed and incubated overnight at 4° C. to allow for hybridization of the aptamer and capture probes. This assay solution was removed and the DHA assay plate was washed with TBST as previously described. 100 μL of the streptavidin-poly-HRP diluted 1:20,000 with DPBS was added to the DHA assay plate, the plate was then sealed and incubated at room temperature for 1 h while shaking. The Streptavidin-Poly-HRP solution was removed and the plate was again washed with TBST and 100 μL of the TMB substrate solution (at RT) was added to each well and the plates were incubated at room temperature for 5 minutes until sufficient colour had developed. 100 μL of the stop solution was added to each well and then the plate was read at 450 nm within 30 minutes after stopping the reaction. Data analysis was performed using Prism 5 for each plate, the background A450 value (0 pM aptamer) was subtracted from all the values within the sample set. The capture probe for DHA was ARC35141 (SEQ ID NO:15):

5′ NH2 mU mU mU mU mC mA mG mA mC mU mA mC
mC mU mA 3′

The detection probe for DHA was ARC35156 (SEQ ID NO:12):

5′ mG mA mU mU mU mC mC mC mU mU mU mU mU
biotin 3′

Oligo-Protein Precipitation Assay:

To confirm aptamer quantitation from the DHA, a new assay was developed called the oligo-precipitation “OP”. This assay uses the same biotinylated capture primers from the DHA to mix with the 100 μL of digested skin samples (epidermis or dermis). The samples are heated and cooled to allow primer to hybridize. Streptavidin beads are added to pull down the aptamer-primer complex. The aptamer was then eluted off the beads loaded onto a 15% urea gel and stained with Sybr Gold to visualize and compare aptamer bands.

Results

It was discovered that, after several months of storage, the cream vehicle used in this study based on the Mehta, et al. is not physically stable, thus not a developable formulation. Therefore, future studies use cream vehicles with known stability.

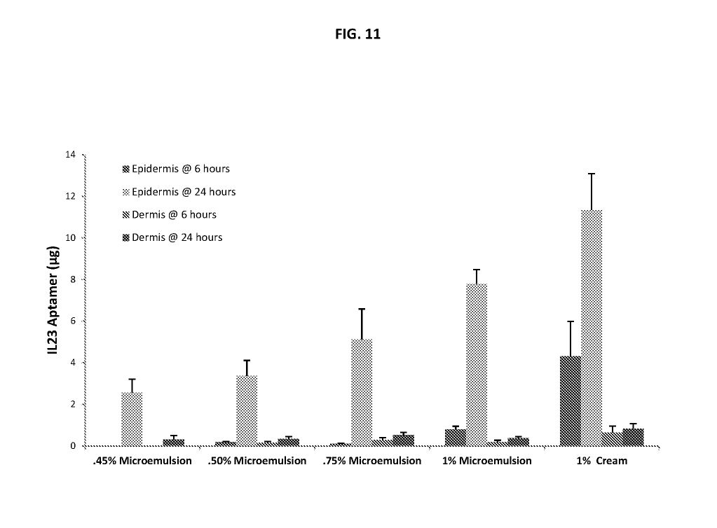

The amount of IL23 aptamer quantified in the epidermis and dermis using the DHA from the different formulations can be found in FIG. 11 and the comparison to IC50 values can be found in Table 5. The microemulsions (0.45%, 0.5%, 0.75%, & 1%) showed a non-linear dose response delivery to the epidermis at 24 hours. An increase in the amount of the IL23 aptamer was shown from 6 to 24 hours for all formulations. The microemulsions showed the greatest increase from 6 to 24 hours with >10-fold increase in the epidermis and >1.9-fold increase in the dermis. This may suggest a longer lag phase for the microemulsions while the cream may be delivering more API in the first hours of application. The 1% cream showed the greatest delivery (1.5-5.5-fold) compared to the microemulsions. At 6 hours, the 1% cream appeared to deliver >3.4-fold more IL23 aptamer (p<0.06) to the skin (epidermis & dermis) and >1.5 fold more IL23 aptamer (p<0.07) compared to the skin (epidermis & dermis) at 24 hours. The 1% cream delivered >0.6 μg of the IL23 aptamer into the dermis and >4.3 μg in the epidermis at both 6 and 24 hours after topical application. The IC₅₀ for the IL23 aptamer in STAT3 activation of primary human T-cells is approximately 300 pM. The cream formulation is delivering 120,000 fold above the IC₅₀ in the epidermis at 24 hours and >3,000 fold above the IC50 at both 6 and 24 hours in the dermis. This suggests 1% cream is delivering significantly above the therapeutic amounts to the skin from a topical vehicle without barrier disruption. The cream formulation contains approximately 60% water compared to <20% for the microemulsion. The IL23 aptamer was shown to have high water solubility, thus one of the delivery mechanisms may be through the hydration of the stratum corneum to increase penetration.

Table 6 shows the skin penetration of IL23 aptamer in human ex vivo skin as percent (%) applied dose. As shown therein, meaningful amounts of aptamer permeate into the epidermis and dermis by 6 hours and this increases through 24 hours post administration.

In order to confirm the values obtained from the DHA, the oligo-precipitation assay was used to extract the aptamer from the tissue and confirmed by Sybr Gold to visualize and compare aptamer bands. The bands aligned with the IL23 standards and the overall intensities correlated where the sample from the cream showed to be darker compared to the microemulsion, confirming the values from the DHA.

FIG. 11 shows the skin penetration of IL23 aptamer in human ex vivo skin. Ex vivo abdominal skin was treated topically with 20 uL (200 ug/cm²) of IL23 Aptamer in a microemulsion or cream formulation. IL23 Aptamer was extracted from the epidermis and dermis at 6 hours and 24 hours post application. Bars represent the mean amount of IL23 Aptamer from 5 replicates on 1 skin donor at 6 hours with zero hour values subtracted or 5 replicates on 2 skin donors for the microemulsion or 5 replicates on 3 skin donors for the cream at 24 hours. Samples were analyzed by DHA shown as mean±SEM.

TABLE 5
Skin Penetration of IL23 Aptamer in Human Ex vivo Skin above IC₅₀. The
amount of IL23 aptamer delivered into the epidermis and dermis 6 and 24 hours post-
application. Values represent the amount IL23 converted to μM from >5 replicates and 1-3
donors (n ≧ 5) ± SEM. Samples were analyzed by DHA with a range from 10 × 10⁻¹² to
40 × 10⁻¹²M. Volume assumptions were 1.0 cm² dosing area with epidermal
thickness of 0.015 cm and an absence of cells (i.e., liquid). The skin volume = 0.015 cm³.
The molecular wt of IL23 aptamer is 20,395.27. The IC₅₀ of IL23 aptamer in primary
T-cells is ≈300 pM (0.0003 μM).

| | 6 hours post-application | | | | 24 hours post-application | | | |
|---|---|---|---|---|---|---|---|---|
| | | Mean | Std Err | | | Mean | Std Err | |
| Formulation | N | (μM) | (μM) | Fold > IC50 | N | (μM) | (μM) | Fold > IC50 |

Epidermis

| .45% Microemulsion | 0 | No Analysis | | | 6 | 8.4 | 2.1 | 27,858 |
|---|---|---|---|---|---|---|---|---|
| .50% Microemulsion | 5 | 0.6 | 0.1 | 1,954 | 16 | 11.0 | 2.4 | 36,747 |
| .75% Microemulsion | 5 | 0.4 | 0.1 | 1,182 | 16 | 16.7 | 4.8 | 55,716 |
| 1% Microemulsion | 5 | 2.6 | 0.5 | 8,583 | 10 | 25.5 | 2.2 | 84,976 |
| 1% Cream | 5 | 14.1 | 5.5 | 46,990 | 16 | 37.1 | 5.7 | 123,659 |

Dermis

| .45% Microemulsion | 0 | No Analysis | | | 6 | 0.4 | 0.3 | 1,416 |
|---|---|---|---|---|---|---|---|---|
| .50% Microemulsion | 5 | 0.2 | 0.1 | 738 | 16 | 0.5 | 0.2 | 1,619 |
| .75% Microemulsion | 5 | 0.4 | 0.2 | 1,309 | 16 | 0.7 | 0.2 | 2,442 |
| 1% Microemulsion | 5 | 0.3 | 0.1 | 895 | 10 | 0.5 | 0.1 | 1,705 |
| 1% Cream | 5 | 0.9 | 0.4 | 3,010 | 16 | 1.2 | 0.3 | 3,873 |

TABLE 6
Skin Penetration of IL23 Aptamer in Human Ex vivo Skin as
percent (%) applied dose. The amount of IL23 aptamer
delivered into the epidermis and dermis 6 and 24 hours
post-application. Values represent the amount IL23 aptamer
converted to % of applied dose from >5 replicates and
1-3 donors (n ≧ 5). Samples were analyzed by DHA with
a range from 10 × 10⁻¹² to 40 × 10⁻¹²M. Formulations
were applied at 10 μL/cm² and were corrected for
weight assuming 1 μL = 1 mg.

| | | 6 Hours | | 24 Hours | |
|---|---|---|---|---|---|
| | Applied | Epidermis | Dermis | Epidermis | Dermis |
| | Dose | % Applied | % Applied | % Applied | % Applied |
| Formulation | (μg) | Dose | Dose | Dose | Dose |
| .45% | 45 | — | — | 5.7% | 0.7% |
| Microemulsion | | | | | |
| .50% | 50 | 0.4% | 0.3% | 6.7% | 0.7% |
| Microemulsion | | | | | |
| .75% | 75 | 0.1% | 0.4% | 6.8% | 0.7% |
| Microemulsion | | | | | |
| 1% | 100 | 0.8% | 0.2% | 7.8% | 0.4% |
| Microemulsion | | | | | |
| 1% Cream | 100 | 4.3% | 0.6% | 11.3% | 0.8% |

Experiments were also performed to evaluate the effects of non-aqueous vehicles on IL23 aptamer permeation into human skin in a Franz cell set up. A water vehicle (97.9% DI water, 0.1% polysorbate 20, 1% benzoyl alcohol, all % w:w), 100% glycerol, and 100% ethanol as a vehicle for aptamer delivery were evaluated. A dose of 10 mg/cm2 of aptamer was used. The receiving fluid was PBS. Epidermis and dermis tissue levels of IL23 aptamer at 6 and 15 hrs were quantified by DHA. At both time points and under all three conditions, aptamer was detected in the epidermis and dermis, with greater amounts in the epidermis at both time points (data not shown). Thus, the IL23 aptamer can permeate into the epidermis and dermis in non-aqueous vehicles and in an aqueous vehicle.

Additional experiments were performed to evaluate the effects of additional vehicles on IL23 aptamer permeation into human skin in a Franz cell set up. A water vehicle (97.9% DI water, 0.1% polysorbate 20, 1% benzoyl alcohol, all % w:w), 1:1 glycerol:DI water, and 1:1 ethanol:DI water as a vehicle for aptamer delivery were evaluated. A dose of 10 mg/cm2 of aptamer was used. The receiving fluid was PBS. Epidermis and dermis tissue levels of IL23 aptamer at 6 and 15 hrs were quantified by DHA. At both time points and under all three conditions, aptamer was detected in the epidermis and dermis (data not shown). Thus, the IL23 aptamer can permeate into the epidermis and dermis in these additional vehicles.

### Example 10: Proof of Concept (POC) Vehicles (Foams, Solutions, and Creams)

The use of penetration enhancers and formulation optimization is commonly used to improve the delivery of small molecules and some of these excipients may also be advantageous in improving the delivery of macromolecules (Williams and Barry (2004) Adv Drug Deliv Rev 56(5):603-18; Karande et al. (2004) Nat Biotechnol 22(2):192-7). The cream was shown to deliver higher amount of IL23 aptamer (ARC32225) into human skin compared to a microemulsion, suggesting there may be opportunities to further improve the penetration of the IL23 aptamer. To build on the water concentration hypothesis, IL23 aptamer (ARC32225) was formulated in an aqueous vehicle (97.9% water), foam solution (82% water), and a cream (62.5% water). To explore the effects of penetration enhancers (PE), IL23 aptamer (ARC32225) was formulated in a glycol solution (15% water and 78% PE).

The aim of this study was to assess the passive permeation of IL23 aptamer (ARC32225) in uncompromised skin from a range of water based creams and solutions that could be potentially progressed to a clinical situation.

Methods

Freshly excised human abdominal skin was dermatomed to a thickness of 500±100 μm and mounted on flow-through diffusion cells using donor blocks to provide a leak proof seal, exposing a surface area of 1.0 cm2. Diffusion cells were connected to multi-channel pumps with a flow rate of approximately 1.8 mL/hr with PBS. Each cell was then equilibrated in a heating manifold to ensure a skin surface temperature of 32° C. (for at least 30 min prior to dosing). IL23 aptamer (ARC32225) formulations were applied topically at a dose of 10 uL/cell (10 mg/cm2), which was spread uniformly onto the stratum corneum surface using a positive displacement pipette. Test articles were applied to two separate donors and at least 6 replicates per skin donor. At six hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. The vials were thawed at a later time and analysed using DHA (previously described).

Cream and Solutions:

The formulations used in this study were developed with the intent to provide a physically stable ‘clinical/commercial ready’ formulation that could be progressed to first time in humans (FTIH) studies. The formulations are divided into two groups; oil-in-water creams, and water based solutions. The water based solutions were derived from marketed foam vehicles (Luxiq SME, Tazarotene SME). The foam based solutions represent the vehicles before propellant would be added to create the final foam product in a closed container system. The creams were based on several marketed formulations with assumed stability (Sorbolene Cream, Aqueous Cream BP, and Hydrozole Cream). The ‘ISIS based cream’ was included as a bridge to previous studies, as well as an aqueous water control (with a small amount of Tween to allow better wetting of the skin). Additionally a glycol based solution with a large concentration of penetration enhancers (propylene glycol, dimethyl isosorbide, and TRANSCUTOL®) was included to explore the effects of penetration enhancers. The formulation composition for the creams and solutions can be found in Table 9.

Results

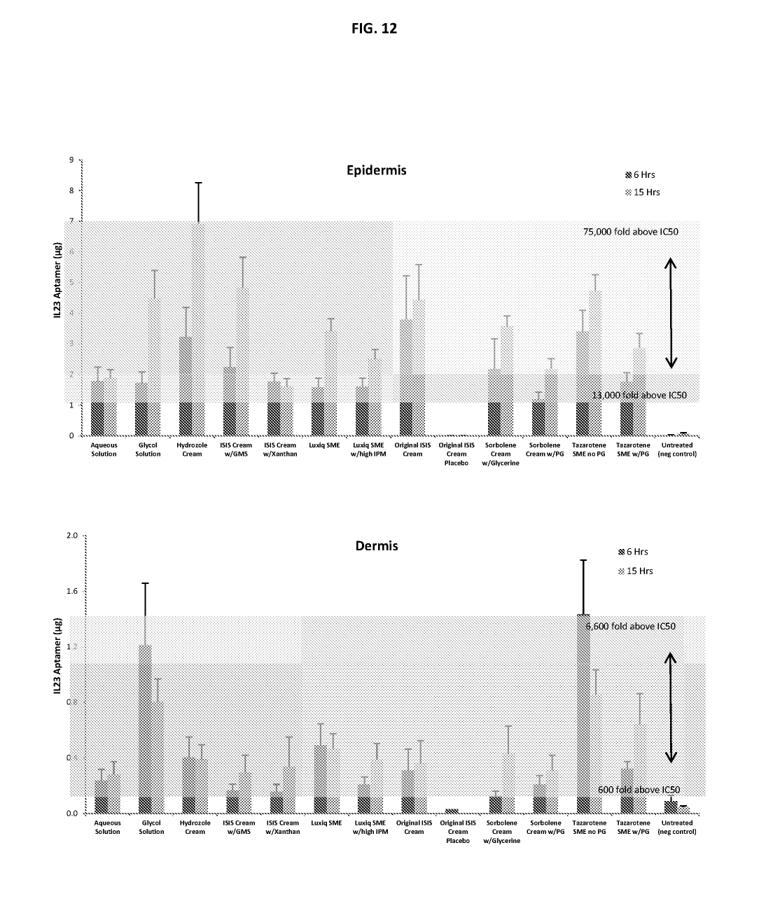

The penetration of IL-23 Aptamer into the epidermis and dermis from 12 prototype active formulations was determined from the epidermis and dermis at six and fifteen hours post application (Table 7 & FIG. 12).

An increase in the delivery of the IL23 aptamer was observed in the epidermis from 6 to 15 hours from all formulations with exception of ISIS Cream w/Xanthan. At 6 hours post application, all but one of the IL-23 aptamer formulations (Sorbolene Cream w/PG) achieved a 13,000 fold or greater increase in the IC₅₀, and by 15 hours all IL-23 aptamer formulations achieved this increase. The amount of IL-23 aptamer recovered from the epidermis was higher than from the dermis at both the t=6 and 15 h time points. The rank order of the top three formulations from the epidermis was Original ISIS Cream>Tazarotene SME no PG>Hydrozole Cream at 6 h, while the top three ranked at 15 h was Hydrozole Cream>ISIS Cream w/GMS>Tazarotene SME no PG. The highest concentration of IL23 observed in the epidermis was from the Hydrozole Cream at 15 hours post-application (6.92±1.35 μg, approximately a 75,000 fold increase above the IC₅₀), which was significantly higher (P<0.05) than the negative control (untreated skin). Although lower at 6 hours post-application (3.23±0.26 ug), the amount of IL-23 aptamer delivered to the epidermis from Hydrazole cream was also significantly higher (P<0.05) than both the Original ISIS Cream Placebo and Untreated skin (negative control). The amount of IL-23 aptamer found in the epidermis from ISIS Cream w/GMS and Sorbolene Cream w/glycerine was significantly higher (P<0.05) than the negative control, but showed no significant improvement (P>0.05) upon the Original ISIS Cream Placebo.

In the dermis, the amount of IL-23 aptamer delivered by these formulations was ranked Tazarotene SME no PG>Glycol Solution>Luxiq SME at 6 h, and remained similar at 15 hours. However Tazarotene SME w/PG replaced Luxiq SME at 15 hours. Conversely to the pattern of amount of IL-23 aptamer observed in the epidermis where the amount increased over time (6 to 15 hours), the amount of IL-23 aptamer in the dermis following application of the top three ranked formulations (Tazarotene SME no PG, Glycol Solution and Luxiq SME) showed the reverse with a higher penetration after 6 hours compared to 15 hours. In addition, the amount of IL-23 aptamer in the dermis was lower than the amount in the epidermis (reaching a maximum of approximately a 6,600 fold increase above the IC₅₀) following the application of Tazarotene SME no PG at six hours (1.43±0.39 ug). All formulations containing IL-23 aptamer achieved a minimum of approximately a 600 fold increase above the IC₅₀.

It is interesting to note that in both the epidermis and dermis samples, the addition of the known penetration enhancer propylene glycol to the Tazarotene SME formulation paradoxically decreased the penetration of IL23 aptamer at both time points, and the same was seen in the formulation Luxiq SME, where the addition of more isopropyl myristate to the formulation had little effect on the amount of IL23 aptamer delivered to the epidermis at 6 hours, but decreased the amount of IL-23 aptamer in the epidermis at 15 hours and in the dermis at both time points.

IL23 aptamer was observed to have penetrated into the both the epidermis and dermis of intact ex vivo human skin as early as 6 hours and continued over 15 hours. The amounts of IL23 aptamer recovered were 13,000-75,000 fold above IC50 and 600-16,000 fold above IC50 in the epidermis and dermis, respectively (see, Table 7). The significant amount of IL-23 aptamer being delivered to the site of action in the dermis provides evidence of the ability of large molecular weight aptamers to penetrate into skin and appear to be at therapeutically relevant levels. It is also shown that certain vehicles may improve this delivery. Both the Glycol Solution and Tazarotene SME without propylene glycol showed ≧4 fold (P>0.001) improved delivery.

Table 8 shows skin penetration of IL23 aptamer in human ex vivo skin as percent (%) applied dose. As shown therein, meaningful amounts of aptamer permeate into the epidermis and dermis by 6 hours and this persists or increases through 15 hours post administration.

TABLE 7
The amount of IL-23 Aptamer delivered into the epidermis and dermis 6 and
15 h post-application. Values represent the average amount from 2 skin donors (3 to 12
replicates) ± SEM. Samples were analysed by DHA.

| | | | | | 15 h | | | |
|---|---|---|---|---|---|---|---|---|
| | 6 h | | | | | | Dermis | |
| | Epidermis | | Dermis | | Epidermis | | (μg ± SEM) | |
| Formulation (short) | N | (μg ± SEM) | N | (μg ± SEM) | N | (μg ± SEM) | N | (μg ± SEM) |
| Aqueous Solution | 12 | 1.79 ± 0.45 | 11 | 0.24 ± 0.08 | 12 | 1.90 ± 0.26 | 10 | 0.28 ± 0.09 |
| Glycol Solution | 12 | 1.73 ± 0.35 | 12 | 1.21 ± 0.45 | 12 | 4.48 ± 0.92 | 12 | 0.80 ± 0.17 |
| Hydrozole Cream | 12 | 3.23 ± 0.96 | 12 | 0.40 ± 0.15 | 12 | 6.92 ± 1.35 | 12 | 0.39 ± 0.10 |
| ISIS Cream w/GMS | 12 | 2.25 ± 0.63 | 11 | 0.17 ± 0.04 | 12 | 4.82 ± 1.00 | 11 | 0.30 ± 0.12 |
| ISIS Cream | 12 | 1.78 ± 0.26 | 12 | 0.16 ± 0.05 | 12 | 1.61 ± 0.27 | 8 | 0.34 ± 0.22 |
| w/Xanthan | | | | | | | | |
| Luxiq SME | 12 | 1.58 ± 0.31 | 12 | 0.49 ± 0.15 | 12 | 3.42 ± 0.40 | 12 | 0.47 ± 0.11 |
| Luxiq SME w/high | 12 | 1.60 ± 0.29 | 12 | 0.21 ± 0.05 | 12 | 2.51 ± 0.31 | 12 | 0.39 ± 0.11 |
| IPM | | | | | | | | |
| Original ISIS Cream | 12 | 3.80 ± 1.42 | 12 | 0.31 ± 0.15 | 12 | 4.42 ± 1.16 | 11 | 0.36 ± 0.16 |
| Original ISIS Cream | 3 | 0.01 ± 0.00 | 1 | 0.03 ± — | 6 | 0.01 ± 0.00 | 0 | — ± — |
| Placebo | | | | | | | | |
| Sorbolene Cream | 12 | 2.18 ± 0.99 | 11 | 0.13 ± 0.03 | 12 | 3.57 ± 0.33 | 11 | 0.43 ± 0.20 |
| w/Glycerine | | | | | | | | |
| Sorbolene Cream | 12 | 1.20 ± 0.23 | 12 | 0.21 ± 0.06 | 12 | 2.17 ± 0.34 | 11 | 0.31 ± 0.11 |
| w/PG | | | | | | | | |
| Tazarotene SME no | 12 | 3.40 ± 0.69 | 12 | 1.43 ± 0.39 | 12 | 4.73 ± 0.52 | 12 | 0.86 ± 0.18 |
| PG | | | | | | | | |
| Tazarotene SME | 12 | 1.76 ± 0.29 | 12 | 0.32 ± 0.05 | 12 | 2.86 ± 0.48 | 12 | 0.64 ± 0.22 |
| w/PG | | | | | | | | |
| Untreated (negative | 11 | 0.02 ± 0.01 | 6 | 0.09 ± 0.04 | 9 | 0.07 ± 0.04 | 3 | 0.05 ± 0.01 |
| control) | | | | | | | | |

FIG. 12 shows the formulation enhancements of skin penetration of IL23 aptamer in human ex vivo skin. Ex vivo abdominal skin was treated topically with 10 uL (100 ug/cm2) of IL23 Aptamer in a Foam, Cream, or Glycol Solution. IL23 Aptamer was extracted from the epidermis and dermis at 6 hours (dark grey) and 15 hours (light grey) post application. Bars represent the mean amount of IL23 Aptamer from 6 replicates on 2 skin donors (n=12). Samples were analyzed by DHA shown as mean±SEM.

TABLE 8
Skin Penetration of IL23 Aptamer in Human Ex vivo Skin as percent (%)
applied dose. The amount of IL23 aptamer delivered into the epidermis and dermis 6 and 15
hours post-application. Values represent the amount IL23 converted to % of applied dose
from 6 replicates and 2 donors (n ≧ 12). Samples were analyzed by DHA with a range from
10 × 10⁻¹² to 40 × 10⁻¹²M. Formulations were applied at 10 μL/cm² and were corrected for
weight assuming 1 μL = 1 mg.

| | | 6 Hours | | 15 Hours | |
|---|---|---|---|---|---|
| | Applied Dose | Epidermis | Dermis | Epidermis | Dermis |
| Formulation | (μg) | % Applied Dose | % Applied Dose | % Applied Dose | % Applied Dose |
| Aqueous Solution | 100 | 1.8% | 0.2% | 1.9% | 0.3% |
| Glycol Solution | 100 | 1.7% | 1.2% | 4.5% | 0.8% |
| Hydrozole Cream | 100 | 3.2% | 0.4% | 6.9% | 0.4% |
| ISIS Cream w/GMS | 100 | 2.2% | 0.2% | 4.8% | 0.3% |
| ISIS Cream w/Xanthan | 100 | 1.8% | 0.2% | 1.6% | 0.3% |
| Luxiq SME | 100 | 1.6% | 0.5% | 3.4% | 0.5% |
| Luxiq SME w/high IPM | 100 | 1.6% | 0.2% | 2.5% | 0.4% |
| Original ISIS Cream | 100 | 3.8% | 0.3% | 4.4% | 0.4% |
| Sorbolene Cream w/Glycerine | 100 | 2.2% | 0.1% | 3.6% | 0.4% |
| Sorbolene Cream w/PG | 100 | 1.2% | 0.2% | 2.2% | 0.3% |
| Tazarotene SME no PG | 100 | 3.4% | 1.4% | 4.7% | 0.9% |
| Tazarotene SME w/PG | 100 | 1.8% | 0.3% | 2.9% | 0.6% |

TABLE 9
Composition of aptamer topical cream and solutions (amounts in % W/W)

| | | [symbol] | [symbol] | | [symbol] | | | | | [symbol] | | |
|---|---|---|---|---|---|---|---|---|---|---|---|---|
| | | [symbol] | [symbol] | [symbol] | [symbol] | | | | | [symbol] | | |
| | | [symbol] | [symbol] | [symbol] | [symbol] | | | | | [symbol] | | |
| | | [symbol] | [symbol] | [symbol] | [symbol] | | | [symbol] | | [symbol] | | |
| [symbol] | [symbol] | [symbol] | [symbol] | [symbol] | [symbol] | | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | [symbol] |
| [symbol] | | [symbol] | | | | | | | | | | |
| [symbol] | | | | | | | | [symbol] | [symbol] | | | |
| [symbol] | [symbol] | [symbol] | [symbol] | | [symbol] | [symbol] | [symbol] | [symbol] | | | [symbol] | |
| [symbol] | [symbol] | [symbol] | | | | | | | | | | |
| [symbol] | | | [symbol] | | | | | | | | | |
| [symbol] | | | | [symbol] | [symbol] | | | | | [symbol] | | |
| [symbol] | [symbol] | [symbol] | [symbol] | | | | | | | | | |
| [symbol] | | | | | | [symbol] | [symbol] | | | | | |
| [symbol] | | | | [symbol] | | [symbol] | | | | [symbol] | | |
| [symbol] | | | | | | | | | [symbol] | | | |
| [symbol] | | | | [symbol] | [symbol] | [symbol] | [symbol] | [symbol] | | [symbol] | | |
| [symbol] | | | | [symbol] | [symbol] | [symbol] | [symbol] | | | [symbol] | | |
| [symbol] | | | | | | | | [symbol] | [symbol] | | | |
| [symbol] | | | | | | [symbol] | [symbol] | | | | | |
| [symbol] | | | | | | [symbol] | [symbol] | | | | | |
| [symbol] | | | | | | | | | [symbol] | | | |
| [symbol] | [symbol] | | | | | | | | | | | |
| [symbol] | | | | [symbol] | | | | | | | | |
| [symbol] | | | | [symbol] | | | | | | | | |
| [symbol] | | [symbol] | | | [symbol] | [symbol] | [symbol] | [symbol] | [symbol] | [symbol] | | |
| [symbol] | | [symbol] | | | [symbol] | [symbol] | [symbol] | [symbol] | [symbol] | [symbol] | | |
| [symbol] | [symbol] | | [symbol] | | | | | | | | | |
| [symbol] | | | | [symbol] | | | | | | | | |
| [symbol] | | | | | [symbol] | [symbol] | [symbol] | [symbol] | | [symbol] | [symbol] | |
| [symbol] | | [symbol] | [symbol] | [symbol] | [symbol] | [symbol] | [symbol] | | | [symbol] | | |
| [symbol] | | [symbol] | [symbol] | [symbol] | [symbol] | | | [symbol] | | [symbol] | [symbol] | [symbol] |
| [symbol] | | | | | | | | | [symbol] | | | |
| [symbol] | | | | | | | | | [symbol] | | | |
| [symbol] | | | | | | | | | | | [symbol] | |
| [symbol] | | | | | | | | | | | [symbol] | |
| Total | 100 | 100 | 100 | 100 | 100 | 100 | 100 | 100 | 100 | 100 | 100 | 100 |

[symbol]

indicates data missing or illegible when filed

### Example 11: Permeation Routes and Aptamer Kinetics

To determine the route and the kinetics of the passive permeation of the IL23 aptamer (ARC32225) into and through the skin, flux experiments were performed using isolated epidermis.

Methods

Epidermal membranes were prepared by immersing full thickness skin in hot deionised water (60±3° C.) for 45 seconds. The epidermal membranes were removed from the underlying dermis using a gloved finger and the dermis was discarded. The epidermal membrane was then floated SC side up in deionised water onto filter paper until the time of experimentation. Epidermal membranes were mounted on flow-through diffusion cells using donor blocks to provide a leak proof seal, exposing a surface area of 1.0 cm². Diffusion cells were connected to multi-channel pumps with a flow rate of approximately 0.3 mL/hr with PBS. Each cell was then equilibrated in a heating manifold to ensure a skin surface temperature of 32° C. (for at least 30 min prior to dosing). IL23 aptamer (ARC32225) formulations were applied topically at a dose of 10 uL/cell (10 mg/cm²), which was spread uniformly onto the stratum corneum surface using a positive displacement pipette. Test articles were applied to two separate donors and at least 16 replicates per skin donor. The receiving fluid was collected every two hours for twenty four hours in 96 well plates and immediately frozen. The plates were later thawed and the concentration of IL23 aptamer in the phosphate buffered saline (PBS) receiving fluid was quantified using DHA (previously described).

Results

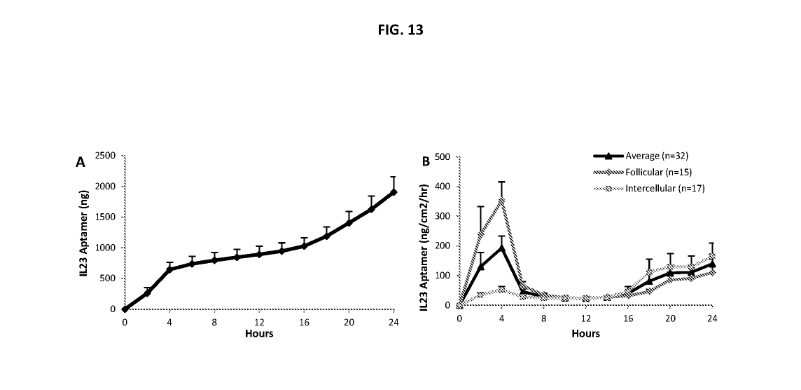

The cumulative amount of IL23 aptamer (ARC32225) over 24 hours shows a rapid initial penetration of IL23 aptamer into the receiving fluid during the first four hours with no apparent lag phase (FIG. 13). This rapid delivery is followed by a slower penetration of the IL23 Aptamer (ARC32225) over the following twelve hours, which is more consistent with a pseudo-steady state profile but also lacks an observable lag phase. When the data is presented as flux (ng/cm²/hr) for each individual flux profile, a peak was observed at 4 hours with a rapid decrease at the subsequent six hour time collection and then resumed a steady state delivery of the IL23 Aptamer (ARC32225) into the receiving fluid over the following ten to twelve hours (FIG. 13). When epidermal membranes are prepared from full thickness skin the hair shaft can become dislodged during isolation resulting in essentially an open channel for follicular delivery. This could explain the rapid delivery observed in some of the samples as shown from the line labeled “Follicular” (FIG. 13). These samples show marked decrease at the following six hour time point suggesting this is not an open channel for sustained penetration. The second line labelled “Intercellular” shows the passive penetration of the IL23 Aptamer (ARC32225) through the stratum corneum, which could be occurring through the intercellular or intracellular pathways in the stratum corneum. The passive delivery of the IL23 Aptamer (ARC32225) appears to be a combination of the follicular and intercellular routes, with the primary route over the duration of this study being the passive delivery through the intercellular or intracellular pathways of the stratum corneum. The total amount penetrating over 24 hours into the receiving fluid was 1.9 μg or approximately 2% of applied dose.

FIG. 13 shows IL23 aptamer (ARC32225) in aqueous vehicle over 24 hours was measured in the receiving fluid after a single topical application on epidermal skin sections shown as cumulative amount (A) and flux (B). Line represents the mean cumulative amount of IL23 Aptamer from 16 replicates on 2 skin donors (n=32). Samples were analyzed by DHA shown as mean±SEM.

### Example 12: Water Gradients and Passive Delivery of IL23 Aptamer

The IL23 aptamer (ARC32225) has been shown to penetrate intact human skin passively at therapeutically relevant levels. There appears to be some vehicle effects, with a water based vehicle showing significant concentrations of IL23 aptamer within the dermis as early as six hours after a single topical application. This is particularly surprising given the large molecular weight and the overwhelming lack of supporting data in the literature for passive delivery of large molecules. It has been shown that RNA aptamers have a conformational plasticity where these molecules are in a constant state of flux in attempts to maintain the lowest energy state [6, 10]. This flexibility may contribute to how this large molecular weight molecule is permeating the skin, but it does not fully explain the mechanisms or pathways for this delivery. Two potential pathways or driving forces that may influence or help explain the delivery are based on unique ion and water gradients that occur in the skin.

The passive permeation from the initial experiments was observed in a water vehicle as well as more clinically viable vehicles. Vehicles with a higher water concentration led to an enhanced delivery of the IL23 aptamer into the skin. The skin has a unique water gradient estimated to be about 15% at the stratum corneum and increases to approximately 80% at the basal membrane [11]. This difference in water content from the surface of the skin to the interior creates an osmotic gradient that could serve as a driving force for large molecules [12]. The IL23 aptamer has shown to have high water solubility. By combining these observations, it was hypothesized that the osmotic gradient in combination with aptamers' conformational plasticity and water solubility could play a role in the passive delivery of the IL23 aptamer.

Methods

Freshly excised human abdominal skin was dermatomed to a thickness of 250±100 μm and mounted on Franz diffusion cells with an average surface area of approximately 0.6 cm² and a volume of approximately 2.0 mL. The dermatomed skin was mounted between the donor and receiver compartments and the cells were sealed together using Parafilm® and clips. The receiver compartments were filled with receiver fluid (water, MS grade). Previous solubility results show water will not impact sink conditions. Each cell was then equilibrated to ensure a surface temperature of 32° C. (for at least 30 min prior to dosing). An additional Franz cell was also mounted but not dosed (to act as a blank control) to assess interference with sample quantification. IL23 aptamer (ARC32225) was dissolved as 1% w/w in an aqueous solution (97.9% w/w deionised water, 0.1% w/w Polysorbate 20 and 1.0% w/w benzyl alcohol). IL23 aptamer (ARC32225) formulations were applied topically at a dose of either a finite dose of 6 mg (i.e. 10 mg/cm²) or an infinite dose of 180 mg (i.e. 300 mg/cm²), which was spread uniformly onto the stratum corneum surface using a positive displacement pipette. Cells that were occluded post treatment had the donor compartment covered with PARAFILM®. Cells with no occlusion will have the donor compartment left open. To hydrate the skin, 1 mL of water (LCMS Grade) was dispensed into the Franz cell donor chamber and Franz cells were placed in a water bath for 12 h at 37° C. prior to application of Test Items. Test articles were applied to one skin donors and at least 4 replicates per skin donor. At six and fifteen hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. The vials were thawed at a later time and analyzed using DHA (previously described).

Results

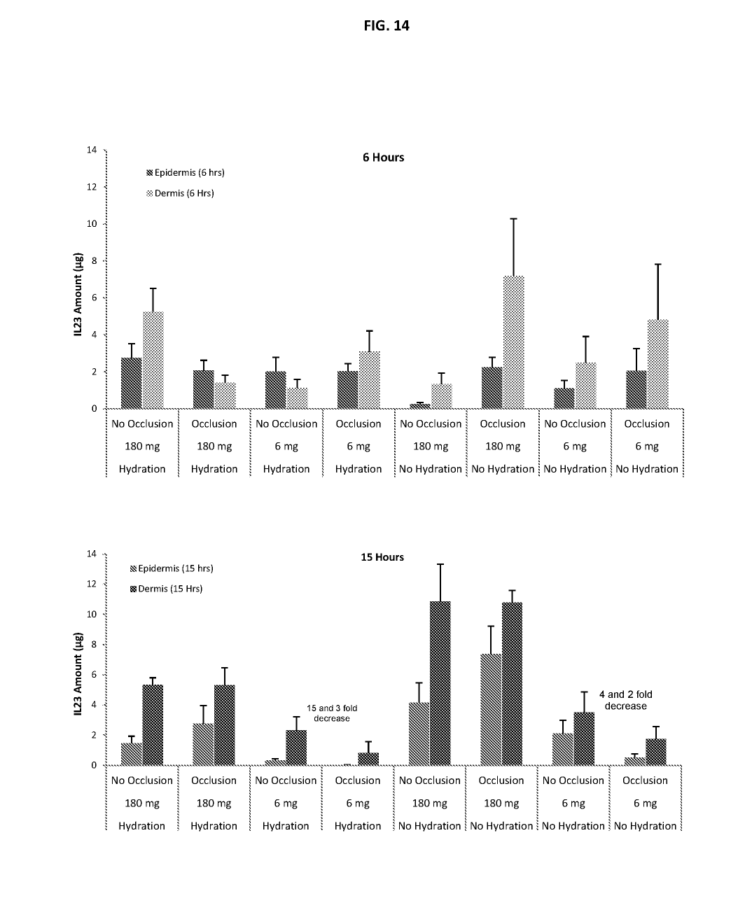

The result for the effects of skin hydration and occlusion from the finite dose (6 mg or 10 mg/cm2) on the passive delivery of IL23 aptamer in ex vivo skin for 6 and 15 hours post-application respectively can be found in FIG. 14. The results for the passive delivery of IL23 aptamer comparing the effects of hydration, occlusion, and finite (6 mg or 10 mg/cm2) vs infinite (180 mg or 300 mg/cm2) for 6 and 15 hours post-application can also be found in FIG. 14.

Hydrating the skin decreased the amount of IL23 aptamer (ARC32225) delivered into dermis at 6 hours post-application by approximately two fold. Interestingly, there didn't appear to be any differences in the amount of IL23 aptamer (ARC32225) delivered to the epidermis. By 15 hours hydration had a similar negative impact on the amount of IL23 aptamer (ARC32225) delivered into the dermis by about two fold. The decrease in delivery impacted by hydration was more pronounced at 15 hours in the epidermis with six fold decrease in the unoccluded skin sections, while the occluded skin sections showed a twenty-three fold reduction in the epidermis. The occluded skin that was hydrated by a pre-treatment of water resulted in very little aptamer in the epidermis. It is unclear why this did not translate to less IL23 aptamer (ARC32225) delivered deeper in the skin to the dermal layers or why this trend was only observed in later time points of 15 hours vs the earlier 6 hour time points. It could not be explained by IL23 aptamer (ARC32225) penetrating into the receiving fluid unless the levels were below the detection limits of the DHA.

The highest levels of IL23 aptamer (ARC32225) were achieved from the infinite doses (180 mg or 300 mg/cm2) in the dermis at 15 hours for the unhydrated skin. When comparing the finite to infinite dosing, the larger doses led to a 2-4 fold increased delivery of IL23 aptamer (ARC32225) to the skin (epidermis or dermis) at 15 hours. These effects seemed to be more pronounced in the 15 hour skin that was not hydrated. Similar to the finite dosing, hydration of the skin decreased amount of IL23 aptamer (ARC32225) in the epidermis and dermis by about two fold, but was more obvious at the 15 hour time point.

FIG. 14 shows passive permeation of IL23 aptamer (ARC32225) comparing the effects of occlusion, hydration, and finite versus infinite dosing. The amount of IL23 aptamer (ARC32225) delivered into the epidermis and dermis 6 and 15 hours post-application is shown. Bars represent the mean amount IL23 Aptamer (ARC32225) from 4 replicates and 1 donors (n=4)±SEM. Samples were analyzed by DHA with a range from 10×10¹² to 40×10⁻¹² M.

### Example 13: Ion Gradients in Skin

In a study with a human IgG RNA aptamer, the active structure was destabilized with EDTA solution, where divalent cations were removed by a chelating agent, and quickly refolded by adding the divalent cations again [10]. These divalent cations may be important in the folding of active aptamer conformational structure and these observations of rapid folding and unfolding suggest the conformational plasticity may be a one of the contributing factors for the passive delivery through the skin barrier. This is of particular interest as there is a distinct gradient of calcium ions (Ca²⁺) across the epidermis, which plays an important role in maintaining the homeostasis of the epidermis [13]. The gradient peaks within the upper stratum granulosum and then sharply declines across the SC [14]. Therefore, it is possible that these unique gradients within the SC could play a factor in the topical delivery of the aptamers. Based on the observations from Nomura, et al., where Ca²⁺ was able to dissociate the IgG RNA aptamer binding by altering the conformational structure, this study explores EDTA containing solvent systems to bind divalent cations (i.e., Ca²⁺, Mg, etc). The IL23 aptamer (ARC32225) was applied in either water vehicle, an EDTA vehicle, or calcium chloride vehicle. The receiving fluid mirrored the dosing vehicle, for example the EDTA vehicle also had the same concentration of EDTA in the receiving fluid directly beneath the skin.

Methods

Freshly excised human abdominal skin was dermatomed to a thickness of 250±100 μm and mounted on Franz diffusion cells with an average surface area of approximately 0.6 cm² and a volume of approximately 2.0 mL. The dermatomed skin was mounted between the donor and receiver compartments and the cells were sealed together using Parafilm® and clips. The receiver compartments were filled with receiver fluid (water, MS grade). Previous solubility results show water will not impact sink conditions. Each cell was then equilibrated to ensure a surface temperature of 32° C. (for at least 30 min prior to dosing). An additional Franz cell was also mounted but not dosed (to act as a blank control) to assess interference with sample quantification. To explore the effect of both calcium and other ion gradients, a 10 mM EDTA solution was prepared and a 10 mM CaCl2 solution was prepared with IL23 aptamer as a topical vehicle and compared to IL23 in a water vehicle without calcium or EDTA. The receiving fluid matched the dosing vehicles (i.e., 10 mM EDTA vehicle and 10 mM EDTA receiving fluid). IL23 aptamer (ARC32225) formulations were applied topically at a dose of 6 mg (i.e. 10 mg/cm²), which was spread uniformly onto the stratum corneum surface using a positive displacement pipette. Test articles were applied to one skin donors and at least 4 replicates per skin donor. At six and fifteen hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. The vials were thawed at a later time and analyzed using DHA (previously described).

Results

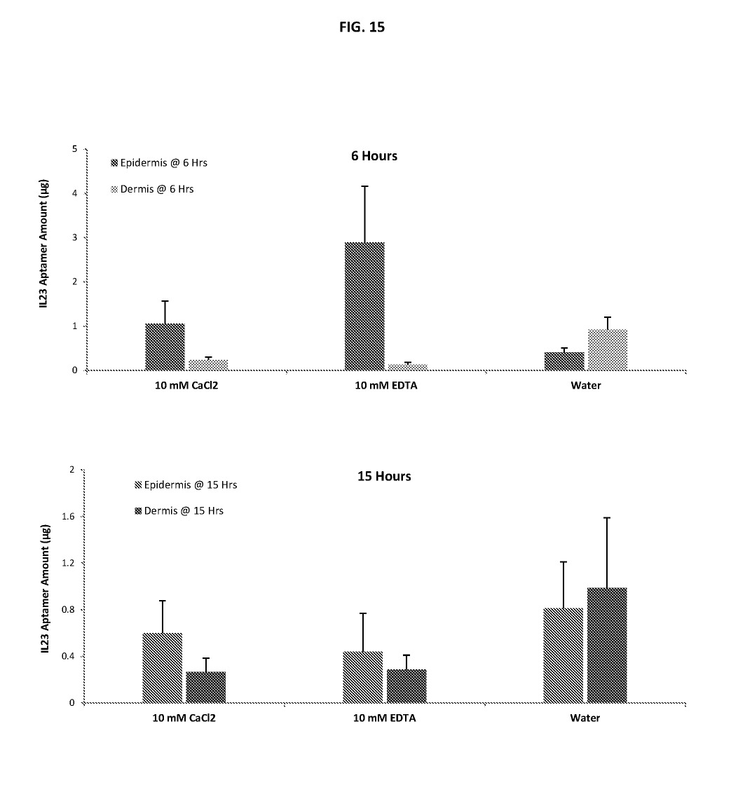

The results for the effects of EDTA from the finite dose (6 mg or 10 mg/cm2) on the passive delivery of IL23 aptamer in ex vivo skin for 6 and 15 hours post-application can be found in FIG. 15. The inclusion of EDTA in the vehicle and receiving fluid did not appear to have a significant impact on the permeation of IL23 aptamer into the skin. The inclusion of calcium in the vehicle and receiving fluid also did not appear to have a significant impact on the permeation of IL23 aptamer into the skin.

FIG. 15 shows passive permeation of IL23 aptamer comparing the effects of EDTA or CaCl2 from a finite dose of aptamer (10 mg/cm2). The amount of IL23 aptamer delivered into the epidermis (darker grey) and dermis (lighter grey) 6 and 15 hours post-application is shown. Bars represent the amount IL23 Aptamer from 4 replicates and 1 donors (n=4)±SEM. Samples were analyzed by DHA with a range from 10×10⁻¹² to 40×10⁻¹² M.

Experiments were also performed under similar conditions but looking at the effects of 10 mM or 100 mM EDTA in the vehicle. IL23 aptamer permeated into the epidermis and dermis at both EDTA concentrations (data not shown).

The effects of EDTA pretreatment (less than 5 minutes) (10 mM EDTA) of the skin were also evaluated. IL23 aptamer permeated into the epidermis and dermis after EDTA pretreatment (data not shown).

### Example 14: Passive Permeation and Active Transport

MARCO, scavenger receptor (SR-A subclass II), was recently shown to be responsible for Herpes Simplex Virus type 1 (HSV-1) uptake and infection in human keratinocytes [15]. In a poster presentation at 2014 Society for Investigative Dermatology this receptor showed positive staining in the stratum corneum [16]. The presence of a scavenger receptor in the stratum corneum was counter to what is widely accepted for this barrier as a means for passive transportation and complete absence of any active transport. Experiments were performed to evaluate whether the IL23 aptamer (ARC32225) was permeating the skin by active or passive transport.

Methods

Freshly excised human abdominal skin was dermatomed to a thickness of 250±100 μm and mounted on Franz diffusion cells with an average surface area of approximately 0.6 cm² and a volume of approximately 2.0 mL. The dermatomed skin was mounted between the donor and receiver compartments and the cells were sealed together using Parafilm® and clips. The receiver compartments were filled with receiver fluid (water, MS grade). Previous solubility results show water will not impact sink conditions. Each cell was then equilibrated to ensure a surface temperature of 32° C. (for at least 30 min prior to dosing). An additional Franz cell was also mounted but not dosed (to act as a blank control) to assess interference with sample quantification. IL23 aptamer (ARC32225) was dissolved as 1% w/w in an aqueous solution (97.9% w/w deionised water, 0.1% w/w Polysorbate 20 and 1.0% w/w benzyl alcohol). IL23 aptamer (ARC32225) formulations were applied topically at a dose of 6 mg (i.e. 10 mg/cm²), which was spread uniformly onto the stratum corneum surface using a positive displacement pipette. Test articles were applied to one skin donors and at least 4 replicates per skin donor. At six and fifteen hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered IL23 aptamer (ARC32225) not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. The vials were thawed at a later time and analyzed using DHA (previously described).

Results

The results for the effects of temperature and epidermis from the finite dose (6 mg or 10 mg/cm2) on the passive delivery of IL23 aptamer in ex vivo skin for 6 and 15 hours post-application can be found in FIG. 16. IL23 aptamer permeated the skin at both 6 and 15 hours for the skin that was kept at either 32° C. or 4° C. The amount of IL23 aptamer found in the skin increased from 6 to 15 hours for the skin that was kept at 32° C. Temperature did not impact the permeation of IL23 aptamer into the skin, thus ruling out the concept of active transport and further supporting the permeation is passive.

FIG. 16 shows passive permeation of IL23 aptamer comparing the effects of temperature a finite dose (10 mg/cm2). The amount of IL23 aptamer delivered into the epidermis and dermis 6 and 15 hours post-application is shown. Bars represent the meant amount IL23 aptamer from 4 replicates and 1 donors (n=4)±SEM. Samples were analyzed by DHA with a range from 10×10⁻¹² to 40×10⁻¹² M.

### Example 15: Effects of Chemical and Structural Modifications on IL23 Aptamer Topical Delivery

Chemical modifications are often made into the nucleotide sugars or internucleotide phosphodiester linkages by replacing the 2′ position with either a fluoro- (F), amino- (NH2) or O-methyl (OCH3) group for enhanced nuclease resistance (Keefe et al. (2010) Nat Rev Drug Discov 9(7):537-50) To determine if different 2′ modifications had an impact on the topical delivery of IL23 aptamer, 2′ methyl or 2′ fluoro modifications were made to the IL23 aptamer. One major difference between antisense oligonucleotides and aptamers are the conformational structures and flexibility (Nomura, Y., et al., (2010) Nucleic Acids Res 38(21):7822-9). Structure in antisense molecules is actively avoided because it can have an impact on efficacy. One of the advantages of aptamers is that structure can be modified and still maintain activity. The IL23 aptamer is folded with the phosphate backbone predominating the exterior of the molecule and the majority of the bases are tied up in secondary structures. To better understand if conformational structure has an impact on delivery for topical delivery of the IL23 aptamers, point mutations were made to the IL23 aptamer that were predicted to change the secondary and tertiary structure. Finally, the broadness of aptamer permeation was explored from large pooled random libraries to help understand different physiochemical properties of aptamers in general.

Methods

Freshly excised human abdominal skin was dermatomed to a thickness of 500±100 μm and mounted on flow-through diffusion cells using donor blocks to provide a leak proof seal, exposing a surface area of 1.0 cm². Diffusion cells were connected to multi-channel pumps with a flow rate of approximately 1.8 mL/hr with PBS. Each cell was then equilibrated in a heating manifold to ensure a skin surface temperature of 32° C. (for at least 30 min prior to dosing). IL23 aptamer (ARC32225) and the IL23 analogues were dissolved as 1% w/w in an aqueous solution (97.9% w/w deionised water, 0.1% w/w Polysorbate 20 and 1.0% w/w benzyl alcohol). IL23 aptamer (ARC32225) and the variants were dissolved in 100% DI water. The aptamers were applied topically at a dose of 10 uL/cell (10 mg/cm²), which was spread uniformly onto the stratum corneum surface using a positive displacement pipette. Test articles were applied to one skin donors and two to seven replicates per skin donor. At six and twenty four hours post-application, the surface of the skin was washed using five cotton swabs soaked in a 0.05% v/v Tween 20/PBS solution. The washed skin was immediately followed by three tape strips applied to the surface of the skin to remove residual formulation from and the first layers of the stratum corneum. These samples were considered aptamer not penetrating the skin and were not analyzed. Following the removal of the residual topical formulations from the surface of the skin, the remaining epidermis and dermis was placed into an oven at 60° C. for 2 min. The epidermis and dermis were removed from the oven and manually separated using forceps and gloved fingers. The epidermal and dermal layers were placed into separate vials and immediately frozen. The vials were thawed at a later time and analyzed using DHA (previously described).

IL23 Variants:

Variants 1-4 contain point mutations that disrupt the 2° structure, while variant 5 contains a mutation that disrupts the tertiary contact point. The mutations were limited to portions of the aptamer that would not disturb the binding of the probes necessary for the DHA. The 3′ and 5′ ends needed to be conserved to allow the quantification of the aptamer using the DHA. The aptamer variants were dissolved in 100% DI water, which was later found not to completely cover the dosing area of the skin due to surface tension from the lipids in the stratum corneum.

Variant 1 (2° structure disruption):
(SEQ ID NO: 20)
mA-fG-fG-fG-mA-mA-mA-fU-fC-fA-mG-fG-fC-fU-
fU-fU-mA-fU-fC-mG-mG-mC-fC-fG-fC-fC-mG-fC-
fU-fC-fC-fC-fU-mG-mU-mG-mC-fC-mA-fU-fC-mG-
fU-mC-mC-fG-mA-mG-mA-mG-fU-mA-mG-mG-fU-mA-
mG-fU-fC-fU-mG.
Variant 2 (2° structure disruption):
(SEQ ID NO: 21)
mA-fG-fG-fG-mA-mA-mA-fU-fC-fA-mG-fG-fC-fU-
fU-fU-mA-fU-fC-mG-mG-mG-mA-fG-fC-fC-mG-fC-
fU-fC-fC-fC-fU-mG-mU-mG-mC-fC-mA-fU-fC-mG-
fU-mC-mC-fG-mA-mG-mA-mG-fU-mA-mG-mG-fU-mA-
mG-fU-fC-fU-mG.
Variant 3 (2° structure disruption):
(SEQ ID NO: 22)
mA-fG-fG-fG-mA-mA-mA-fU-fC-fA-mG-fG-fC-fU-
fU-fU-mA-fU-fC-mG-mG-mG-fC-mA-fC-fC-mG-fC-
fU-fC-fC-fC-fU-mG-mU-mG-mC-fC-mA-fU-fC-mG-
fU-mC-mC-fG-mA-mG-mA-mG-fU-mA-mG-mG-fU-mA-
mG-fU-fC-fU-mG.
Variant 4 (2° structure disruption):
(SEQ ID NO: 23)
mA-fG-fG-fG-mA-mA-mA-fU-fC-fA-mG-fG-fC-fU-
fU-fU-mA-fU-fC-mG-mG-mG-fC-fG-fC-fC-mG-fC-
fU-fC-fC-fC-fU-mG-mU-mG-mC-fC-mA-fU-fC-mG-
fU-mC-mC-fG-mA-mG-mA-mG-fU-mC-mG-mG-fU-mA-
mG-fU-fC-fU-mG.
Variant 5 (3° structure disruption):
(SEQ ID NO: 24)
mA-fG-fG-fG-mA-mA-mA-fU-fC-fA-mG-fG-fC-fU-
fU-fU-mA-fU-fC-mG-mG-mG-fC-fG-fC-fC-mG-fC-
fU-mA-fC-fC-fU-mG-mU-mG-mC-fC-mA-fU-fC-mG-
fU-mC-mC-fG-mA-mG-mA-mG-fU-mA-mG-mG-fU-mA-
mG-fU-fC-fU-mG.

Aptamer Pooled Library:

fCmD structured pool with 2′-fluoro-C; 2′-methoxy-A,G,U composition at 2.5 uM. fGmH structured pool with 2′-fluoro-G; 2′-methoxy-A,C,U composition at 2.5 uM. fRmY with 2′-fluoro-A, G; 2′-methoxy-C,U at 541 uM. rGmH pool with 2′-hydroxy-G; 2′-methoxy-A,C,U at 549 uM.

IL23 Analogues (Modifications):

MNA IL23 analogue (uniformly 2′-OMe ribose) methyl group is added to the 2′ hydroxyl group at 12.15 mg/mL dissolved in vehicle containing 97.9% DI Water, 0.1% Polysorbate 20, 1% Benzoyl alcohol. fCmD IL23 Analogue (2′-fluoro Cytidine; 2′-OMe Adenosine, Guanosine, Uridine) at 3.29 mg/mL dissolved in vehicle containing 97.9% DI Water, 0.1% Polysorbate 20, 1% Benzoyl alcohol. fGmH IL23 Analogue (2′-fluoro Guanosine; 2′-OMe Adenosine, Cytidine, Uridine) at 10.87 mg/mL dissolved in vehicle containing 97.9% DI Water, 0.1% Polysorbate 20, 1% Benzoyl alcohol.

Results

IL23 2′ Modifications:

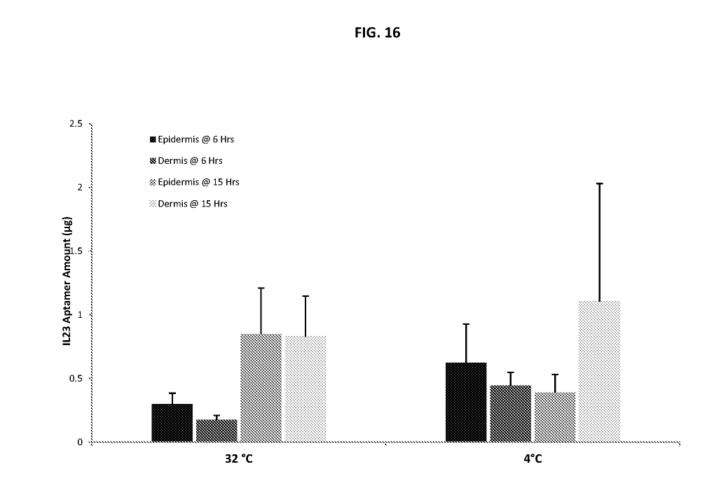

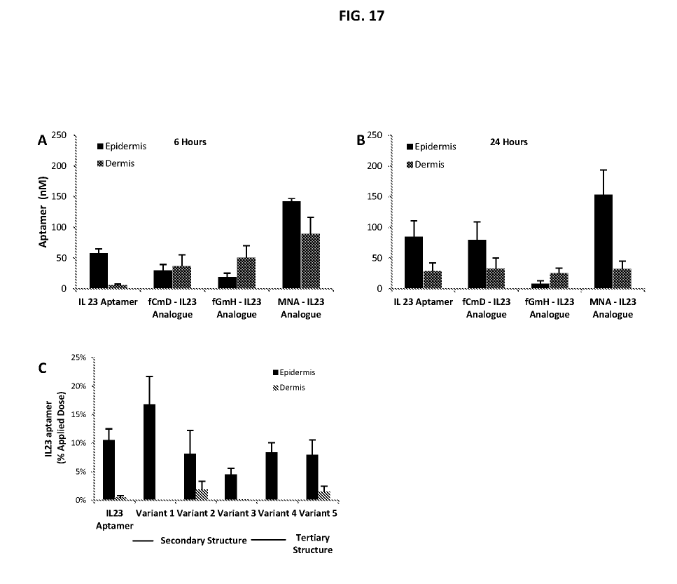

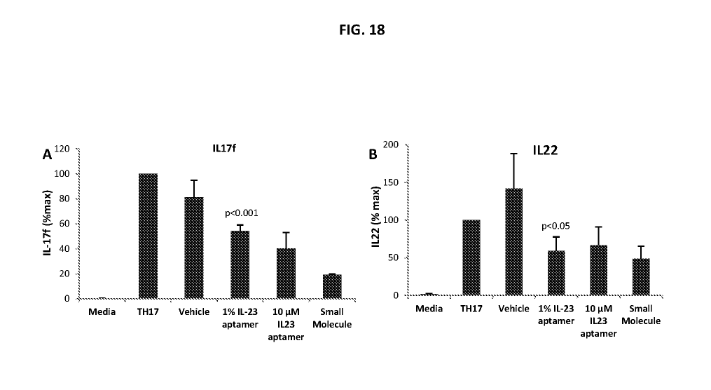

The IL23 aptamers with the different 2′ modifications all penetrated the epidermis and dermis as early at six hours and twenty-four hours at therapeutic levels (560 to 34,100-Fold above IC50) (FIG. 17). The MNA IL23 aptamer analogue showed the highest amount of aptamer in the epidermis at both 6 and 24 hours, suggesting methoxylation may have a positive correlation for better skin penetration (FIG. 17). These methoxylated compounds may have different solubility which could impact the thermodynamics of the compound and ultimately its ability to penetrate the skin.

A DNA-based IL23 aptamer was also prepared. However, the ability of this aptamer to penetrate into the epidermis and dermis could not be evaluated due to the inability of the DHA to detect this aptamer, which was confirmed with lack of signal from a standard curve.

Table 10 shows skin penetration of IL23 aptamer in human ex vivo skin as percent (%) applied dose. As shown therein, meaningful amounts of aptamer permeate into the epidermis and dermis by 6 hours and increase or persist through 15 hours post administration.

TABLE 10
Skin Penetration of 2′ modified IL23 aptamers in Human Ex vivo
Skin as percent (%) applied dose. The amount of 2′ modified
IL23 aptamers delivered into the epidermis and dermis 6 and 15
hours post-application in aqueous solution. Values represent the
amount IL23 converted to % of applied dose from 2-7 replicates
and 1 donors. Samples were analyzed by DHA with a range
from 10 × 10⁻¹² to 40 × 10⁻¹²M. Formulations were applied at
10 μL/cm² and were corrected for weight assuming 1 μL = 1 mg.

| | | 6 Hours | | 15 Hours | |
|---|---|---|---|---|---|
| | Applied | Epidermis | Dermis | Epidermis | Dermis |
| Formulation | Dose | % Applied | % Applied | % Applied | % Applied |
| (short) | (μg) | Dose | Dose | Dose | Dose |
| IL 23 Aptamer | 100 | 1.2% | 0.1% | 1.7% | 0.6% |
| fCmD - | 100 | 0.6% | 0.8% | 1.6% | 0.7% |
| IL23 Analogue | | | | | |
| fGmH - | 100 | 0.4% | 1.0% | 0.2% | 0.5% |
| IL23 Analogue | | | | | |
| MNA - | 100 | 2.9% | 1.8% | 3.1% | 0.7% |
| IL23 Analogue | | | | | |

Structural Alterations (Variants):

All four of the variants with changes in the secondary structure showed significant concentrations in the epidermis over 24 hours with 4-16% of applied dose penetrating the skin (FIG. 17). Variant 5, where point mutations disrupted or changed the tertiary structure, also seemed to penetrate the epidermis over 24 hours (FIG. 17). Variant 1 and Variant 4 did not result in detectable levels in the dermis. The overall low levels in the skin compared to the previous studies is likely explained by the water vehicle that did not adequately wet the skin due to the surface tension between the vehicle and the lipids in the stratum corneum. It is recommended to include a surfactant (i.e., polysorbate 20) in aqueous vehicle to break this surface tension. Modifying the secondary or tertiary structure of the IL23 aptamer did not have a major impact on the penetration, suggesting that the ability of this aptamer is not completely dependent on structure.

Aptamer Pooled Libraries:

The fCmD, fGmH, fRmY and rGmH aptamer libraries were extracted from proteinase K digested skin samples using biotinylated hybridization probes and subjected to qPCR. The average number of aptamers recovered from each skin sample was over 200 billion with an average recovery rate of 0.051%. These large recovered amounts suggest that aptamers as a class of molecules penetrates skin.

Because the amount that was recovered was so large, it was not possible to sample the entire sequence space. However, the fCmD and fGmH and fRmY and rGmH libraries were sequenced for enrichment (>2 copies), where the data was processed and analyzed to only store sequences that are considered enriched from a statistical perspective. These results show selection of 5.1 million sequences were generated and of these only 252 were enriched. However, these enriched sequences are largely primer:dimers and artifacts. A sequence that is preferred over others at the recovery rate seen would provide greater than two sequences. For example, the dermis sample from the fRmY library showed a recovery of 3.9e+11 molecules with a recovery rate of 0.024%. The number of sequences from this dermis sample was 351,241. If there were a preference for a particular sequence at that recovery rate, one would expect to see 84 sequences.

Approximately 5.1 million sequences were found only once or twice in the total dataset showing there was no preference for any particular sequence. If aptamers with specific chemistry (sequence or composition) resulting in better penetration, it would be expected to have more copies in the sequencing data. This lack of difference in sequencing also suggests the penetration of aptamer is a general trait of these molecules. The recovery rates in each library are shown in Table 11.

TABLE 11
Library recovery rates for test articles as measured by qPCR.

| | | | Molecules | Molecules/test | % |
|---|---|---|---|---|---|
| Description | Library | Comp | Recovered (qPCR) | article | Recovery |
| Chamber 1D epidermis | ARC31812 | fCmD | 2.55E+10 | 1.51E+13 | 0.1691 |
| Chamber 1D dermis | ARC31812 | fCmD | 1.91E+10 | 1.51E+13 | 0.1266 |
| Chamber 1E epidermis | ARC31812 | fCmD | 2.71E+10 | 1.51E+13 | 0.1799 |
| Chamber 1E dermis | ARC31812 | fCmD | 1.31E+10 | 1.51E+13 | 0.0870 |
| Chamber 1F epidermis | ARC31812 | fCmD | 3.19E+10 | 1.51E+13 | 0.2118 |
| Chamber 1F dermis | ARC31812 | fCmD | 1.72E+10 | 1.51E+13 | 0.1140 |
| Chamber 1G epidermis | ARC31812 | fGmH | 1.42E+09 | 1.51E+13 | 0.0095 |
| Chamber 1G dermis | ARC31812 | fGmH | 1.48E+09 | 1.51E+13 | 0.0099 |
| Chamber 1H epidermis | ARC31812 | fGmH | 3.01E+09 | 1.51E+13 | 0.0200 |
| Chamber 1H dermis | ARC31812 | fGmH | 1.31E+09 | 1.51E+13 | 0.0087 |
| Chamber 1I epidermis | ARC31812 | fGmH | 3.80E+08 | 1.51E+13 | 0.0025 |
| Chamber 1I dermis | ARC31812 | fGmH | 7.50E+08 | 1.51E+13 | 0.0050 |
| Chamber 2B epidermis | ARC20508 | fRmY | 3.905E+11 | 1.63E+15 | 0.0240 |
| Chamber 2B dermis | ARC20508 | fRmY | 3.905E+11 | 1.63E+15 | 0.0240 |
| Chamber 2C epidermis | ARC20508 | fRmY | 3.905E+11 | 1.63E+15 | 0.0240 |
| Chamber 2C dermis | ARC20508 | fRmY | 3.905E+11 | 1.63E+15 | 0.0240 |
| Chamber 2D epidermis | ARC20508 | fRmY | 2.715E+11 | 1.63E+15 | 0.0167 |
| Chamber 2D dermis | ARC20508 | fRmY | 3.905E+11 | 1.63E+15 | 0.0240 |
| Chamber 2E epidermis | ARC20511 | rGmH | 2.155E+11 | 1.65E+15 | 0.0130 |
| Chamber 2E dermis | ARC20511 | rGmH | 2.155E+11 | 1.65E+15 | 0.0130 |
| Chamber 2F epidermis | ARC20511 | rGmH | 3.905E+11 | 1.65E+15 | 0.0236 |
| Chamber 2F dermis | ARC20511 | rGmH | 3.905E+11 | 1.65E+15 | 0.0236 |
| Chamber 2G epidermis | ARC20511 | rGmH | 3.905E+11 | 1.65E+15 | 0.0236 |
| Chamber 2G dermis | ARC20511 | rGmH | 9.175E+11 | 1.65E+15 | 0.0555 |

FIG. 17 shows skin penetration of different 2′ modified IL23 aptamers on ex vivo human skin. Ex vivo abdominal skin was treated topically with 10 μL (IL23 108 ug/cm², MNA 121.5 ug/cm², fCmD 32.9 ug/cm², and fGmH 108.7 ug/cm²) of four different 2′ modified IL23 Aptamers in an Aqueous Solution. Bars represent the mean amount of IL23 Aptamer from 2-7 replicates on a single skin donor at 6 hours (A) and 24 hours (B). Ex vivo abdominal skin was treated topically with 10 uL (Variant 1; 1.1 mg/mL, Variant 2; 0.4 mg/mL, Variant 3; 0.9 mg/mL, and Variant 4; 0.9 mg/mL) of four different variants disrupting secondary structure and one variant (Variant 5; 1.1 mg/mL) disrupting tertiary structure and IL23 aptamer (1.0 mg/mL) in a water solution. Bars represent the amount IL23 Aptamer from 4 replicates on a single skin donors (n≧4)±SEM at 24 hours (C). The amount of IL23 Aptamer delivered into the epidermis (black bars) and dermis (grey bars). Samples were analyzed by DHA shown as mean±SEM.

### Example 16: Biological Activity in Human Skin from Therapeutic Aptamers

In order to confirm the therapeutic concentrations of IL23 aptamer in the dermis from all the skin penetration studies were biologically active, freshly excised human skin was mounted and clamped in place using static cells containing growth media and stimulated to induce a Th17 response. The IL23 aptamer was formulated in a water vehicle (97.9% w/w deionised water, 0.1% w/w Polysorbate 20 and 1.0% w/w benzyl alcohol), applied topically, and compared to IL23 aptamer and an ROR gamma inverse agonist in the media.

Methods

Th17 Stimulated Human Ex-Vivo Skin Model

Tissue Culture Using Static Cells:

Freshly excised healthy human skin was dermatomed to 750 um and cleaned with antibiotic/antimycotic solution made up as 1% GIBCO™ Antibiotic-Antimycotic (100×), 0.1% Gentamicin in 1× Dulbecco's Phosphate Buffered Saline. Twelve mm diameter biopsies were cut using disposable single-use biopsy punches (Acupunch, Acuderm, Inc.) and washed in antibiotic/antimycotic solution for 5-10 minutes. Skin biopsies were placed on autoclaved 7 mm (0.38 cm²) unjacketed static cells with 2 mL receptor volume (PermeGear, Inc; No. 6G-01-00-07-02) and leak proof seal was maintained using metal clamps and donor chamber. Receptor chamber was filled with cornification using pastuer pipette to dispense in sampling port. Static cells were then placed in a humidified incubator at 37° C. On Day 1 (Day-1) test articles were dosed topically or systemically in the media. Twenty-four hours later (Day 0), the media was replaced and the Th17 stimulation ‘cocktail’ was added to the receptor chamber of the Franz cells. Twenty-four hours later (Day 1) the tissue was removed minced to less than 1×1×1 mm pieces and stored in 10× volume of RNA later (Qiagen, Cat#76104) with 300 μL of RNeasy Lysis Buffer (Qiagen, Cat#79216) supplemented with 1% 2-Beta-Mercapto-Ethanol for RNA isolation.

Cornification Media:

Media consisted of 237 mL of Dulbecco's Modified Eagle Medium (DMEM) (GIBCO®, Cat#11995-065), 237 mL Ham's F-12K (Kaighn's) Medium (GIBCO®, 21127-022), 1 mL 90 mM Adenine, 1 mL 0.94M CaCl, 1 mL 10 nM Tri-iodothyronine, 1 mL Insulin-Transferrin-Selenium-Ethanolamine (ITS-X) (100×) (GIBCO® 5100-056), 5 mL Antibiotic-Antimycotic (100×) (GIBCO® 15240-062), 10 mL Fetal Bovine Serum (FBS) (HyClone™, SH30071.01HI), 5 mL GlutaMAX™ Supplement (GIBCO®, 35050-061), 0.1 mL 50 mg/ml Gentamicin (Invitrogen, #15750060).

Th17 Stimulation Cocktail:

The cytokine cocktail was optimized to simulate naive CD4 T-cells, prevent stimulation of TH1 and TH2 cells, and focus the stimulation of TH17 cells. Naive CD4 stimulation was achieved through a combination of 1 ug/ml purified NA/LE Mouse anti-human CD3 (BD Pharmingen, Cat#555329) and 2 ug/ml Human CD28 Antibody (R&DSystems, Cat# MAB342). The prevention of TH1 and TH2 cells was achieved by combining 1 ug/ml Human IFN-gamma Antibody (R&DSystems, Cat#MAB2851), 1 ug/ml Human IL-4 Antibody (R&DSystems, Cat# MAB304). Stimulation of TH17 cells was through a mixture of 10 ng/ml Recombinant Human IL-1b/IL-1F2 (R&DSystems, Cat#201-LB-025/CF), 10 ng/ml Recombinant Human IL-6 (R&DSystems, Cat#206-IL-010/CF), 1 ng/ml Recombinant Human TGF-b1 (R&DSystems, Cat#240-B-010/CF), and Recombinant human IL-21 (Southern Biotech, Cat#19000-00). All components incorporated in a single mixture with the cornification media.

RNA Isolation & Quantitation:

Approximately 40 mg of minced tissue was added to homogenization tubes containing 2.8 and 1.4 mm ceramic beads with. The tissue was disrupted using high-throughput bead mill homogenizer (PRECELLYS®24 Atkinson, N.H.) machine at 6300 rpm for 30 seconds and 10 cycles with a 2-minute ice break. The homogenate was digested by adding 490 μL of water containing 10 uL Proteinase K (Thermo Scientific, Cat # E00491) at 55° C. for 15 minutes. Digested tissue was spun down for 3 minutes at 10,000×g to pellet cell debris and the supernatant was used for RNA isolation using Qiagen's Mini RNA Isolation kit (Cat #74106) according to manufacturer's protocol. Total RNA was quantified using Nanodrop 2000 (Thermo Scientific, Wilmington, Del.). Isolated RNA (1.4 ug) from skin tissue was used as a template in a 20 uL PCR volume using Invitrogen SuperScript VILO cDNA Synthesis kit (#11754-050) to create cDNA template. The cDNA was diluted 1:25 for subsequent qPCR with the specific TaqMan probe for each gene to be quantified. Life Technologies AVii7 PCR machine was used for the qPCR 40 amplification cycles. RNA levels of GAPDH, IL17A, IL17F, & IL22 relative expression were calculated using the Delta Delta CT formula.

Results

The Th17 stimulating conditions resulted in significant upregulation of both IL17f and IL22 (FIG. 18). The topical application of IL23 aptamer resulted in a decrease of both IL17f and IL22 (p<0.001 and 0.05 respectively) and these reductions were similar to IL23 aptamer included in the media. Interestingly, the reduction of IL22 from the topical application of IL23 aptamer was similar compared to small molecule ROR gamma inverse agonist. This confirms the therapeutic levels quantified previously are of a conformational structure that is capable of binding to extracellular IL23 within the skin's microenvironment at biologically meaningful levels.

FIG. 18 shows that topical application of IL23 aptamer inhibits Th17 derived cytokines in human skin. Freshly excised human abdominal skin was mounted and clamped in place using static cells containing growth media and stimulated twenty four hours later to induce a Th17 response. The skin was treated topically twice with 8 μL (210 μg/cm2) before and after Th17 stimulation. 10 μM of IL23 aptamer and an RORgamma inverse agonist was included in the media as systemic controls. Twenty-four hours post stimulation skin was harvested and relative transcript levels of Th17-type cytokines, IL17f and IL22 were determined by qPCR. Bars represent the mean percent of maximum stimulation (set to 100%) from 3 replicates on 3 different skin donors (n=9).

### REFERENCES

1. Watkinson, A. C., A commentary on transdermal drug delivery systems in clinical trials. J Pharm Sci., 2013. 102(9): p. 3082-3088.
2. Nemes, Z. and P. M. Steinert, Bricks and mortar of the epidermal barrier. Exp Mol Med, 1999. 31(1): p. 5-19.
3. Bos, J. D. and M. M. Meinardi, The 500 Dalton rule for the skin penetration of chemical compounds and drugs. Exp Dermatol, 2000. 9(3): p. 165-9.
4. Bouwstra, J. A., Gooris, G. S., The Lipid Organisation in Human Stratum Corneum and Model Systems. The Open Dermatology Journal, 2010. 4: p. 10-13.
5. Kim, Y. S. and M. B. Gu, Advances in Aptamer Screening and Small Molecule Aptasensors. Adv Biochem Eng Biotechnol.
6. Nakamura, Y. I., A. Miyakawa, S., RNA plasticity and selectivity applicable to therapeutics and novel biosensor development. Genes Cells, 2012. 17(5): p. 344-64.
7. Russell-Jones, G. and R. Himes, Water-in-oil microemulsions for effective transdermal delivery of proteins. Expert. Opin. Drug Deliv., 2011. 8(4): p. 537-546.
8. Mehta, R. C., et al., Intercellular adhesion molecule-1 suppression in skin by topical delivery of anti-sense oligonucleotides. J Invest Dermatol, 2000. 115(5): p. 805-12.
9. Drolet, D. W., et al., Pharmacokinetics and safety of an anti-vascular endothelial growth factor aptamer (NX1838) following injection into the vitreous humor of rhesus monkeys. Pharm Res, 2000. 17(12): p. 1503-10.
10. Nomura, Y., et al., Conformational plasticity of RNA for target recognition as revealed by the 2.15 A crystal structure of a human IgG-aptamer complex. Nucleic Acids Res, 2010. 38(21): p. 7822-9.
11. Cevc, G. and G. Blume, Lipid vesicles penetrate into intact skin owing to the transdermal osmotic gradients and hydration force. Biochim Biophys Acta, 1992. 1104(1): p. 226-32.
12. Cevc, G., A. Schatzlein, and H. Richardsen, Ultradeformable lipid vesicles can penetrate the skin and other semi-permeable barriers unfragmented. Evidence from double label CLSM experiments and direct size measurements. Biochim Biophys Acta, 2002. 1564(1): p. 21-30.
13. Tanojo H, M. H., Role of calcium ions in relation to skin barrier function., in Percutaneous Absorption: Drugs-Cosmetics-Mechanisms-Methodology., M. H. Bronaugh R L, Editor. 1999. p. 939-950.
14. Elias, P., et al., Origin of the epidermal calcium gradient: regulation by barrier status and role of active vs passive mechanisms. J Invest Dermatol, 2002. 119(6): p. 1269-74.
15. MacLeod, D. T., et al., HSV-1 exploits the innate immune scavenger receptor MARCO to enhance epithelial adsorption and infection. Nat Commun, 2013. 4: p. 1963.
16. Song, Q., Briley, W., Narayan, S., Wang, X., Mirkin, C., Paller, A. S., Epidermal Class A Scavenger Receptors are Critical for Penetration of Topically Delivered Spherical Nucleic Acid Nanoconjugates (SNA) and Gene Suppression, in Society for Investigative Dermatology. 2014: Poster 366.
17. Keefe, A. D., S. Pai, and A. Ellington, Aptamers as therapeutics. Nat Rev Drug Discov, 2010. 9(7): p. 537-50.
18. Immunological Reviews (2008) Vol. 226:103
19. Annu. Rev. Immunol. (2007) Vol. 25:221
20. Trends Immunol. (2006) Vol. 27:17
21. Curr. Med. Chem. (2006) Vol. 13:1149
22. Immunity (2000) Vol. 13:715
23. J. Immunol. (2002) Vol. 168:5699
24. Nat. Rev. Immunol. (2003) Vol. 3:133
25. Nat. Immunol. (2009) Vol. 10:314
26. Nature (2003) Vol. 421:744
27. J. Exp. Med. (2003) Vol. 198:1951
28. Immunol. Lett. (2005) Vol. 96:211
29. Semin. Immunol. (2007) Vol. 19:372
30. J. Clin. Invest. (2006) Vol. 116:1317
31. Curr. Opinion Immun. (2008) Vol. 20:663
32. J. Immun. (2010) Vol. 185:5688
33. Immunity (2010) Vol. 33:279
34. Am. J. Hum. Genet. (2007) Vol. 80:273; Hum. Genet. (2007) Vol. 122:201
35. F1000 Biol. Reports (2010) Vol. 2:40
36. Semin. Cutan. Med. Surg. (2010) Vol. 29:3
37. J. Immunol. (2011) Vol. 186:1495
38. J. Immun. (2010) Vol. 185:5688
39. Ther. Clin. Risk Manag. (2010) Vol. 6:123
40. Cytokine Therapies: Ann. N.Y. Acad. Sci. (2009) Vol. 1182:97
41. Prescrire Int. (2009) Vol. 18:202
42. Semin. Cutan. Med. Surg. (2010) Vol. 29:48
43. J. Invest. Dermatol. (2000) Vol. 115:805

1. A method of treating a skin disease in a subject, the method comprising:
topically administering an effective amount of a dose of a pharmaceutically acceptable composition comprising an aptamer to skin of the subject that comprises the skin disease, wherein the skin comprises epidermis and dermis, wherein at least 0.01% of the applied dose enters into the epidermis of the skin.

2. The method of claim 1, wherein the concentration of the aptamer in the pharmaceutically acceptable composition is between 0.001% and 20%.

3. The method of claim 1, wherein the pharmaceutically acceptable composition is administered as a spray, a liquid, an ointment, a cream, a lotion, a solution, a suspension, an emulsion, a paste, a gel, a powder, a foam, a slow release nanoparticle, a slow release microparticle, a bioadhesive, a patch, a bandage, a semi-solid dosage form, or a wound dressing.

4. The method of claim 1, wherein the pharmaceutically acceptable composition is administered as an aqueous solution.

5. The method of claim 1, wherein the pharmaceutically acceptable composition is administered as a semi-solid dosage form.

6. The method of claim 1, wherein the pharmaceutically acceptable composition is administered as a cream.

7. The method of claim 1, wherein the pharmaceutically acceptable composition does not contain a penetration enhancer.

8. The method of claim 1, wherein the applied dose of the pharmaceutically acceptable composition to the skin is between 0.01 and 20 mg/cm².

9. The method of claim 1, wherein the percentage of the applied dose of topically administered aptamer that enters the epidermis is determined in an in vitro assay using ex vivo human skin.

10. The method of claim 1, wherein the aptamer enters into the dermis of the skin; and wherein at least 0.01% of the applied dose enters into the dermis.

11. The method of claim 10, wherein the percentage of the applied dose of topically administered aptamer that enters the dermis is determined in an in vitro assay using ex vivo human skin.

12. The method of claim 1, wherein less than 5% of the topically applied aptamer reaches systemic circulation.

13. The method of claim 1, wherein the length of the aptamer is less than 100 nucleotides.

14. The method of claim 1, wherein the length of the aptamer is greater than 30 nucleotides.

15. The method of claim 1, wherein the aptamer is between 30 and 90 nucleotides in length.

16. The method of claim 1, wherein at least one nucleotide of the aptamer comprises a chemical modification.

17. The method of claim 16, wherein all nucleotides of the aptamer comprise a chemical modification.

18. The method of claim 16, wherein the chemical modification comprises a modification on the 2′ position of the sugar.

19. The method of claim 16, wherein the chemical modification comprises a 2′-O-methoxyethyl addition.

20. The method of claim 16, wherein the chemical modification comprises a 2′ fluoro addition.

21. The method of claim 1, wherein the aptamer comprises a single inverted deoxythymidine residue (idT) on its 3′ terminus.

22. The method of claim 1, wherein the pharmaceutically acceptable composition is administered once daily.

23. The method of claim 1, wherein the pharmaceutically acceptable composition is administered twice daily.

24. The method of claim 1, wherein the pharmaceutically acceptable composition is administered once weekly.

25. The method of claim 1, wherein the pharmaceutically acceptable composition is administered in combination with a second agent that comprises a treatment for the skin disease.

26. A method for topically administering an aptamer to a subject, the method comprising:
administering a dose of a pharmaceutically acceptable composition comprising an aptamer to skin of the subject, wherein the skin comprises epidermis and dermis;
wherein the aptamer enters into the epidermis of the skin; and
wherein at least 0.01% of the applied dose enters into the epidermis;
thereby topically administering the aptamer to the subject.

27. The method of claim 26, wherein the skin is normal skin.

28. The method of claim 26, wherein the skin is compromised skin.

29. The method of claim 28, wherein the compromised skin is diseased skin.

30. The method of claim 28, wherein the compromised skin comprises an altered barrier.

31. A method of treating a skin disease in a subject, the method comprising topically administering a dose of a pharmaceutically acceptable composition comprising an aptamer to skin of the subject, wherein the skin comprises epidermis and dermis, wherein the aptamer has intrinsic activity in the skin.

32. The method of claim 31, wherein the intrinsic activity is measured as an IC50 value.

33. The method of claim 32, wherein the aptamer is present in the epidermis at a level at least ten-fold above the IC50 of the aptamer.

34. The method of claim 32, wherein the aptamer is present in the dermis at a level at least ten-fold above the IC50 of the aptamer.

35. A method for topically delivering cargo to a subject, the method comprising:
administering a pharmaceutically acceptable composition comprising an aptamer to skin of the subject, wherein the skin comprises epidermis and dermis;
wherein cargo is attached to the aptamer;
wherein the aptamer and attached cargo enter into the epidermis of the skin;
thereby topically delivering cargo to the subject.
